(19) United States
(12) Patent Application Publication
(10) Pub. No.: US 2024/0050462 A1
(43) Pub. Date: Feb. 15, 2024

(54) PHARMACEUTICAL COMPOSITION CONTAINING DOUBLE-STRANDED RIBONUCLEIC ACID INHIBITING EXPRESSION OF COMPLEMENT C5

(71) Applicant: Eisai R&D Management Co., Ltd., Tokyo (JP)

(72) Inventors: Seiji TAKEMOTO, Nagoya (JP); Shuntaro ARASE, Mizuho (JP); Yuta SUZUKI, Tsukuba (JP)

(21) Appl. No.: 17/768,283

(22) PCT Filed: Dec. 24, 2020
(86) PCT No.: PCT/JP2020/048441

§ 371 Date: Apr. 12, 2022

(30) Foreign Application Priority Data
Dec. 26, 2019 (JP) 2019-236918

(51) Int. Cl. A61K 31/713; A61K 9/107; A61K 9/127; A61P 13/02

(52) U.S. Cl. CPC A61K 31/713; A61K 9/107; A61K 9/1272; A61P 13/02

(57) ABSTRACT

Disclosed is a pharmaceutical composition comprising a lipid complex, wherein the lipid complex comprises a double-stranded ribonucleic acid comprising a sense strand consisting of a nucleotide sequence set forth in SEQ ID NO: 145 and an antisense strand consisting of a nucleotide sequence set forth in SEQ ID NO: 146, and a pH of a solution of the lipid complex is 5.0 or less, or 7.5 or more.

### TECHNICAL FIELD

The present invention relates to a pharmaceutical composition comprising a double-stranded ribonucleic acid (dsRNA) capable of suppressing expression of complement C5. More specifically, the present invention relates to a pharmaceutical composition comprising a lipid complex comprising a double-stranded ribonucleic acid capable of suppressing expression of complement C5, a method for producing the pharmaceutical composition, and a method for stabilizing the pharmaceutical composition.

### BACKGROUND ART

A protein group called complement includes proteins indicated as C1 to C9, and these proteins are successively activated through three different pathways (classical pathway, lectin pathway, alternative pathway) to elicit immune response. The fifth complement component, C5, is cleaved to C5a and C5b by C5 convertase. C5a is called anaphylatoxin, and induces inflammatory response for various cells via C5aR (CD88) and C5L2 (GPR77). C5b sequentially reacts with C6 to C9 to be converted into a membrane attack complex (MAC) as a final product, which causes bacteriolysis to pathogens or cell lysis. The complement system may elicit strong cytotoxicity to host cells if the complement system fails to be suitably controlled or is excessively activated.

From previous studies, the complement C5 is known to be associated with various diseases including paroxysmal nocturnal hemoglobinuria (PNH), atypical hemolytic uremic syndrome (aHUS), myasthenia gravis (MG), neuromyelitis optica (NMO), antibody-mediated rejection in kidney transplantation, Guillain-Barre syndrome, antineutrophil cytoplasmic antibody-associated vasculitis (ANCA-associated vasculitis), amyotrophic lateral sclerosis (ALS), Parkinson's disease (PD), autoimmune encephalitis, IgG4-related diseases, asthma, antiphospholipid antibody syndrome, ischemia-reperfusion injury, typical hemolytic uremic syndrome (tHUS), multifocal motor neuropathy (MMN), multiple sclerosis (MS), thrombotic thrombocytopenic purpura (TTP), spontaneous abortion, habitual abortion, traumatic brain injury, cold agglutinin disease, dermatomyositis, hemolytic uremic syndrome associated with Shigatoxin-producing Escherichia coli (E. coli), graft dysfunction, myocardial infarction, sepsis, atherosclerosis, septic shock, spinal cord injury, psoriasis, autoimmune hemolytic anemia (AIHA), antiphospholipid syndrome (APS), myocarditis, immune complex vasculitis, Takayasu's disease, and Kawasaki's disease (arteritis). Thus, inhibition or suppression of expression of complement C5 is expected to lead to successful treatment of these diseases. In particular, inhibition of complement C5 is suggested to be effective for treating or preventing paroxysmal nocturnal hemoglobinuria (Non Patent Literature 1), atypical hemolytic uremic syndrome (Non Patent Literature 2), myasthenia gravis (Non Patent Literature 3), neuromyelitis optica (Non Patent Literature 4), and antibody-mediated kidney transplant rejections (Non Patent Literature 5).

The anti-C5 monoclonal antibody eculizumab (Soliris (registered trademark)) exhibits high affinity for complement C5, and suppresses excessive activation of the complement through inhibition of cleavage of C5 into C5a/C5b and accompanying formation of a membrane attack complex. Thereby, eculizumab exhibits inhibitory effect on hemolysis, and thus is known as a therapeutic agent for paroxysmal nocturnal hemoglobinuria and atypical hemolytic uremic syndrome. In addition, eculizumab is known as a therapeutic agent for generalized myasthenia gravis (gMG). However, eculizumab is very expensive, and hence development of alternative means applicable to treatment and prevention of complement C5-mediated diseases is desired.

Examples of methods for suppressing expression of complement C5 include methods utilizing RNA interference (hereinafter, also referred to as “RNAi”). For example, a double-stranded ribonucleic acid (dsRNA) agent is known, which induces cleavage of an RNA transcript of the C5 gene via an RNA-induced silencing complex (RISC) (Patent Literature 1).

### CITATION LIST

### Patent Literature

[Patent Literature 1] WO 2014/160129

### Non-Patent Literature

[Non-Patent Literature 1] Non Patent Literature 1: Peter Hillmen et al., The New England Journal of Medicine 2004 Feb 5; 350(6): 552-559.

[Non-Patent Literature 2] Legendre CM et al., The New England Journal of Medicine 2013 Jun 6; 368(23): 2169-2181.

[Non-Patent Literature 3] Howard JF Jr et al., Muscle Nerve 2013 July; 48(1): 76-84.

[Non-Patent Literature 4] Pittock SJ et al., The Lancet Neurology 2013 June; 12(6): 554-562.

[Non-Patent Literature 5] Stegall MD et al., American Journal of Transplantation 2011 November; 11(11): 2405-2413.

### SUMMARY OF INVENTION

### Technical Problem

An object of the present invention is to provide a novel pharmaceutical composition comprising a lipid complex comprising a double-stranded ribonucleic acid for suppressing expression of complement C5, a method for producing the pharmaceutical composition, and a method for stabilizing the pharmaceutical composition.

### Solution to Problem

The present invention provides, for example, the following <1> to <81>.
<1> A pharmaceutical composition comprising:
   a lipid complex,
   wherein the lipid complex comprises a double-stranded ribonucleic acid comprising a combination of a sense strand and an antisense strand,
   the combination of the sense strand and the antisense strand is selected from the group consisting of a sense strand consisting of a nucleotide sequence set forth in SEQ ID NO: 159 and an antisense strand consisting of a nucleotide sequence set forth in SEQ ID NO: 160, a sense strand consisting of a nucleotide sequence set forth in SEQ ID NO: 141 and an antisense strand consisting of a nucleotide sequence set forth in SEQ ID NO: 142, a sense strand consisting of a nucleotide sequence set forth in SEQ ID NO: 143 and an antisense strand consisting of a nucleotide sequence set forth in SEQ ID NO: 144, a sense strand consisting of a nucleotide sequence set forth in SEQ ID NO: 145 and an antisense strand consisting of a nucleotide sequence set forth in SEQ ID NO: 146, a sense strand consisting of a nucleotide sequence set forth in SEQ ID NO: 147 and an antisense strand consisting of a nucleotide sequence set forth in SEQ ID NO: 148, and a sense strand consisting of a nucleotide sequence set forth in SEQ ID NO: 153 and an antisense strand consisting of a nucleotide sequence set forth in SEQ ID NO: 154, and
   a pH of a solution of the lipid complex is 5.0 or less or 7.5 or more.
<2> A pharmaceutical composition comprising:
   a lipid complex,
   wherein the lipid complex comprises a double-stranded ribonucleic acid comprising a sense strand consisting of a nucleotide sequence set forth in SEQ ID NO: 145 and an antisense strand consisting of a nucleotide sequence set forth in SEQ ID NO: 146, and
   a pH of a solution of the lipid complex is 5.0 or less or 7.5 or more.
<3> The pharmaceutical composition according to <1>or <2>, wherein the pH of the solution of the lipid complex is 2.0 or more and 5.0 or less, or 7.5 or more and 11.0 or less.
<4> The pharmaceutical composition according to <1>or <2>, wherein the pH of the solution of the lipid complex is 5.0 or less.
<5> The pharmaceutical composition according to <1>or <2>, wherein the pH of the solution of the lipid complex is 2.0 or more and 5.0 or less.
<6> The pharmaceutical composition according to <1>or <2>, wherein the pH of the solution of the lipid complex is 7.5 or more.
<7> The pharmaceutical composition according to <1>or <2>, wherein the pH of the solution of the lipid complex is 7.5 or more and 11.0 or less.
<8> The pharmaceutical composition according to <1>or <2>, wherein the pH of the solution of the lipid complex is 7.5 or more and 10.0 or less.
<9> The pharmaceutical composition according to <1>or <2>, wherein the pH of the solution of the lipid complex is 7.5 or more and 9.5 or less.
<10> The pharmaceutical composition according to <1>or <2>, wherein the pH of the solution of the lipid complex is 7.5 or more and 9.0 or less.
<11> The pharmaceutical composition according to <1>or <2>, wherein the pH of the solution of the lipid complex is 7.5 or more and 8.5 or less.
<12> The pharmaceutical composition according to any one of <1> to <11>, wherein the average particle size of the lipid complex is 100 nm or less.
<13> The pharmaceutical composition according to any one of <1> to <12>, wherein the average particle size of the lipid complex is 65 nm or more and 100 nm or less.
<14> The pharmaceutical composition according to any one of <1> to <12>, wherein the average particle size of the lipid complex is 80 nm or more and 100 nm or less.
<15> The pharmaceutical composition according to any one of <1> to <12>, wherein the average particle size of the lipid complex is 85 nm or more and 100 nm or less.
<16> The pharmaceutical composition according to any one of <1> to <15>, wherein the change in the average particle size of the lipid complex after storage for 2 weeks is 10% or less from the average particle size of the lipid complex before the storage.
<17> The pharmaceutical composition according to any one of <1> to <15>, wherein the change in the average particle size of the lipid complex after storage for 2 weeks is 8% or less from the average particle size of the lipid complex before the storage.
<18> The pharmaceutical composition according to any one of <1> to <15>, wherein the change in the average particle size of the lipid complex after storage for 2 weeks is 5% or less from the average particle size of the lipid complex before the storage.
<19> The pharmaceutical composition according to any one of <1> to <15>, wherein the change in the average particle size of the lipid complex after storage for 1 month is 10% or less from the average particle size of the lipid complex before the storage.
<20> The pharmaceutical composition according to any one of <1> to <15>, wherein the change in the average particle size of the lipid complex after storage for 1 month is 8% or less from the average particle size of the lipid complex before the storage.
<21> The pharmaceutical composition according to any one of <1> to <15>, wherein the change in the average particle size of the lipid complex after storage for 1 month is 5% or less from the average particle size of the lipid complex before the storage.
<22> The pharmaceutical composition according to any one of <1> to <15>, wherein the change in the average particle size of the lipid complex after storage for 3 months is 10% or less from the average particle size of the lipid complex before the storage.
<23> The pharmaceutical composition according to any one of <1> to <15>, wherein the change in the average particle size of the lipid complex after storage for 3 months is 8% or less from the average particle size of the lipid complex before the storage. <24> The pharmaceutical composition according to any one of <1> to <15>, wherein the change in the average particle size of the lipid complex after storage for 3 months is 5% or less from the average particle size of the lipid complex before the storage.
<25> The pharmaceutical composition according to any one of <16> to <24>, wherein the change in the average particle size is increase in the average particle size.
<26> The pharmaceutical composition according to any one of <16> to <25>, wherein a condition for the storage of the pharmaceutical composition is 2 to 8° C.
<27> The pharmaceutical composition according to any one of <16> to <25>, wherein a condition for the storage of the pharmaceutical composition is 25° C.
<28> The pharmaceutical composition according to any one of <1> to <27>, wherein the lipid complex comprises:
   a cationic lipid; and
   at least one lipid selected from the group consisting of neutral lipid, polyethylene glycol-modified lipid, and sterol.
<29> The pharmaceutical composition according to any one of <1> to <28>, wherein the lipid complex comprises a cationic lipid, neutral lipid, polyethylene glycol-modified lipid, and sterol.
<30> The pharmaceutical composition according to <28>or <29>, wherein the cationic lipid is selected from the group consisting of 1-oxo-1-(undecan-5-yloxy)nonadecan-10-yl-1-methylpiperidine-4-carboxylate, 1-((2-butylo ctyl)oxy)-1-oxononadecan-10-yl-1-methylpiperidine-4-carboxylate, 1-oxo-1-(undecan-5-yloxy)heptadecan-8-yl- 1-methylpiperidine 4-carboxylate, 21-oxo-21-(undecan-5 -yloxy)henicosan-10-yl-1-methylpiperidine 4-carboxylate, 21-(octan-3-yl oxy)-21-oxohenicosan-l0-yl-1-methylpiperidine-4-carboxylate, 1-((2-butyloctyl)oxy)-1-oxoicosan-10-yl-1-methylpiperidine-4-carboxylate, (Z)-1-((2-butylnon-3-en-1-yl) oxy)-1-oxoicosan-10-yl-1-methylpiperidine-4-carboxylate, 1-oxo-1-((3-pentyloctyl)oxy)icosan-10-yl-1-methylpiperidine-4-carboxylate, 1-((3,4-dipropylheptyl)oxy)-1-oxoicosan-10-yl-1-methylpiperidine-4-carboxylate, 1-((6-(butyldi sulfanyl)-3-(3-(butyldisulfanyl)propyl)hexyl)oxy)-1-oxoicosan-10-y1-1-methylpiperidine-4-carboxylate, 2 -butyloctyl-104(4-(dimethylamino)butanoyl)oxy)icosanoate, 2-{9-[(2-butyloctyl)oxy] -9-oxononyl}dodecyl1-methylpiperidine-4-carboxylate, 2-{9-oxo-9-[(3-pentyl octyl) oxy]nonyl}dodecyl 1-methylpiperidine-4-carboxylate, 2 -nonyl-11-oxo-11-[(3-pentyloctyl)oxy]undecyl 1-methylpiperidine-4-carboxylate, bis(3-pentyloctyl) 9-{[(1-methylpiperidine-4-carbonyl)oxy]methyl}heptadecanedioate, di [(Z)-2-nonen-l-yl]9-{[(1-methylpiperidine-4-carbonyl)oxy]methyl}heptadecanedioate, 1-(2-octylcyclopropyl)heptadecan-8-yl-1-methylpiperidine-4-carboxylate, (3 S)-2-{9-oxo-9-[(3-pentyloctypoxy]nonyl}dodecyl 1-methylpyrrolidine-3-carboxylate, and (3R)-2-{9-oxo-9-[(3-pentyloctypoxy]nonyl}dodecyl 1-methylpyrrolidine-3-carboxylate.
<31> The pharmaceutical composition according to any one of <28> to <30>, wherein the cationic lipid is selected from the group consisting of 1-((2-butylo ctyl)oxy)-1-oxoic o san-10-yl-1-methylpiper dine-4-carboxylate, 1-((2-butylo ctyl)oxy)-1-oxononadecan- 10-yl-1-methylpiperidine-4-carboxylate, 2-{9-oxo-9-[(3-pentyloctyl)oxy]nonyl}dodecyl 1-methylpiperidine-4-carboxylate, 1-(2-octylcyclopropyl)heptadecan-8-yl-1-methylpiperidine-4-carboxylate, (3 S)-2-{9-oxo-9-[(3-pentyloctypoxy]nonyl}dodecyl 1-methylpyrrolidine-3 -carboxylate, and (3R)-2-{9-oxo-9-[(3-pentyloctypoxy]nonyl}dodecyl 1-methylpyrrolidine-3 -carboxylate.
<32> The pharmaceutical composition according to any one of <28> to <31>, wherein the cationic lipid is 2-{9-oxo-9-[(3-pentyloctypoxy]nonyl}dodecyl 1-methylpiperidine-4-carboxylate.
<33> The pharmaceutical composition according to any one of <28> to <32>, wherein the neutral lipid is phospholipid or ceramide.
<34> The pharmaceutical composition according to <33>, wherein the phospholipid is selected from the group consisting of DOPE, POPE, HSPC, SOPC, POPC, EPC, DMPC, DPPC, DSPC, DAPC, DBPC, DLPC, DOPC, DOPQ DPP DSP DOPS, DOPE-MAL, and sphingomyelin.
<35> The pharmaceutical composition according to <33>or <34>, wherein the phospholipid is selected from the group consisting of DOPE, HSPC, DPPC, DSPC, and DAPC.
<36> The pharmaceutical composition according to any one of <33> to <35>, wherein the phospholipid is DSPC.
<37> The pharmaceutical composition according to any one of <28> to <36>, wherein the polyethylene glycol-modified lipid is selected from the group consisting of PEG2000-DMQ PEG2000-DPQ PEG2000-DSQ PEG5000-DMQ PEG5000-DPQ PEG5000-DSQ PEG-cDMA, PEG-C-DOMG, PEG-DADS PEG-DAA, PEG-phospholipid, PEG-cholesterol, and PEG-ceramide (Cer).
<38> The pharmaceutical composition according to any one of <28> to <37>, wherein the polyethylene glycol-modified lipid is selected from the group consisting of PEG2000-DMQ PEG2000-DPQ PEG2000-DSQ PEG-cDMA, and PEG-C-DOMG.
<39> The pharmaceutical composition according to any one of <28> to <38>, wherein the polyethylene glycol-modified lipid is PEG2000-DMG.
<40> The pharmaceutical composition according to any one of <28> to <39>, wherein the sterol is selected from the group consisting of cholesterol, dihydrocholesterol, lanosterol, β-sitosterol, campesterol, stigmasterol, bras sic asterol, ergosterol, fucosterol, and 3β-[N-(N′,N′-dimethyl amino ethyl) carbamoyl]cholesterol (DC-Chol).
<41> The pharmaceutical composition according to any one of <28> to <40>, wherein the sterol is selected from the group consisting of cholesterol, dihydrocholesterol, lanosterol, and β-sitosterol.
<42> The pharmaceutical composition according to any one of <28> to <41>, wherein the sterol is cholesterol.
<43> The pharmaceutical composition according to any one of <1> to <42>, wherein the lipid complex comprises 2-{9-oxo-9-[(3-pentyloctyl)oxy]nonyl}dodecyl 1-methylpiperidine-4-carboxylate, DSPC, PEG2000-DMQ and cholesterol.
<44> The pharmaceutical composition according to any one of <1> to <43>, wherein the mole ratio of cationic lipid/neutral lipid/polyethylene glycol-modified lipid/sterol in the lipid complex is 30 to 90/0.1 to 20/0.01 to 10/0.1 to 70.
<45> The pharmaceutical composition according to any one of <1> to <43>, wherein the mole ratio of cationic lipid/neutral lipid/polyethylene glycol-modified lipid/sterol in the lipid complex is 40 to 70/3 to 15/0.1 to 3/15 to 60.
<46> The pharmaceutical composition according to any one of <1> to <43>, wherein the mole ratio of cationic lipid/neutral lipid/polyethylene glycol-modified lipid/sterol in the lipid complex is 60/10.5/1.5/28.
<47> The pharmaceutical composition according to any one of <1> to <46>, wherein the lipid complex is a lipid nanoparticle (LNP).
<48> The pharmaceutical composition according to any one of <1> to <47>, wherein the lipid complex encapsulates a double-stranded ribonucleic acid comprising a combination of a sense strand and an antisense strand.
<49> The pharmaceutical composition according to any one of <1> to <48>, further comprising a pharmaceutically acceptable carrier.
<50> The pharmaceutical composition according to any one of <1> to <49>, for treating paroxysmal nocturnal hemoglobinuria.
<51> The pharmaceutical composition according to any one of <1> to <49>, for treating atypical hemolytic uremic syndrome.
<52> A method for treating paroxysmal nocturnal hemoglobinuria, comprising:
   administering the pharmaceutical composition according to any one of <1> to <49> to a patient in need thereof.
<53> A method for treating atypical hemolytic uremic syndrome, comprising:
   administering the pharmaceutical composition according to any one of <1> to <49> to a patient in need thereof.
<54> The pharmaceutical composition according to any one of <1> to <49>, for use in treating paroxysmal nocturnal hemoglobinuria.
<55> The pharmaceutical composition according to any one of <1> to <49>, for use in treating atypical hemolytic uremic syndrome.
<56> A method for producing the pharmaceutical composition according to any one of <1> to <55>, comprising:
   adjusting the pH of the solution of the lipid complex to 5.0 or less or 7.5 or more.
<57> The method according to <56> for producing the pharmaceutical composition according to any one of <1> to <55>, comprising:
   adjusting the pH of the solution of the lipid complex to 2.0 or more and 5.0 or less, or 7.5 or more and 11.0 or less. <58> The method according to <56> for producing the pharmaceutical composition according to any one of <1> to <55>, comprising:
   adjusting the pH of the solution of the lipid complex to 5.0 or less.
<59> The method according to <56> for producing the pharmaceutical composition according to any one of <1> to <55>, comprising:
   adjusting the pH of the solution of the lipid complex to 2.0 or more and 5.0 or less.
<60> The method according to <56> for producing the pharmaceutical composition according to any one of <1> to <55>, comprising:
   adjusting the pH of the solution of the lipid complex to 7.5 or more. <61> The method according to <56> for producing the pharmaceutical composition according to any one of <1> to <55>, comprising:
   adjusting the pH of the solution of the lipid complex to 7.5 or more and 11.0 or less.
<62> The method according to <56> for producing the pharmaceutical composition according to any one of <1> to <55>, comprising:
   adjusting the pH of the solution of the lipid complex to 7.5 or more and 10.0 or less.
<63> The method according to <56> for producing the pharmaceutical composition according to any one of <1> to <55>, comprising:
   adjusting the pH of the solution of the lipid complex to 7.5 or more and 9.5 or less.
<64> The method according to <56> for producing the pharmaceutical composition according to any one of <1> to <55>, comprising:
   adjusting the pH of the solution of the lipid complex to 7.5 or more and 9.0 or less.
<65> The method according to <56> for producing the pharmaceutical composition according to any one of <1> to <55>, comprising:
   adjusting the pH of the solution of the lipid complex to 7.5 or more and 8.5 or less.
<66> The method according to any one of <56> to <65> for producing the pharmaceutical composition according to any one of <1> to <55>, comprising:
   mixing an organic solvent comprising (I) a cationic lipid and (II) at least one lipid selected from the group consisting of neutral lipid, polyethylene glycol-modified lipid, and sterol, and an aqueous solution comprising a double-stranded ribonucleic acid comprising a combination of a sense strand and an antisense strand to obtain a mixed solution.
<67> The method according to <66> for producing the pharmaceutical composition according to any one of <1> to <55>, further comprising:
   removing the organic solvent from the mixed solution.
<68> A method for stabilizing the pharmaceutical composition according to any one of <1> to <55>, comprising:
   adjusting the pH of the solution of the lipid complex to 5.0 or less or 7.5 or more.
<69> The method according to <68> for stabilizing the pharmaceutical composition according to any one of <1> to <55>, comprising:
   adjusting the pH of the solution of the lipid complex to 2.0 or more and 5.0 or less, or 7.5 or more and 11.0 or less.
<70> The method according to <68> for stabilizing the pharmaceutical composition according to any one of <1> to <55>, comprising:
   adjusting the pH of the solution of the lipid complex to 5.0 or less.
<71> The method according to <68> for stabilizing the pharmaceutical composition according to any one of <1> to <55>, comprising:
   adjusting the pH of the solution of the lipid complex to 2.0 or more and 5.0 or less.
<72> The method according to <68> for stabilizing the pharmaceutical composition according to any one of <1> to <55>, comprising:
   adjusting the pH of the solution of the lipid complex to 7.5 or more.
<73> The method according to <68> for stabilizing the pharmaceutical composition according to any one of <1> to <55>, comprising:
   adjusting the pH of the solution of the lipid complex to 7.5 or more and 11.0 or less.
<74> The method according to <68> for stabilizing the pharmaceutical composition according to any one of <1> to <55>, comprising:
   adjusting the pH of the solution of the lipid complex to 7.5 or more and 10.0 or less.
<75> The method according to <68> for stabilizing the pharmaceutical composition according to any one of <1> to <55>, comprising:
   adjusting the pH of the solution of the lipid complex to 7.5 or more and 9.5 or less.
<76> The method according to <68> for stabilizing the pharmaceutical composition according to any one of <1> to <55>, comprising:
   adjusting the pH of the solution of the lipid complex to 7.5 or more and 9.0 or less.
<77> The method according to <68> for stabilizing the pharmaceutical composition according to any one of <1> to <55>, comprising:
   adjusting the pH of the solution of the lipid complex to 7.5 or more and 8.5 or less.
<78> The method according to any one of <68> to <77> for stabilizing the pharmaceutical composition according to any one of <1> to <55>, comprising:
   mixing an organic solvent comprising (I) a cationic lipid and (II) at least one lipid selected from the group consisting of neutral lipid, polyethylene glycol-modified lipid, and sterol, and an aqueous solution comprising a double-stranded ribonucleic acid comprising a combination of a sense strand and an antisense strand to obtain a mixed solution.
<79> The method according to <78> for stabilizing the pharmaceutical composition according to any one of <1> to <55>, further comprising:
   removing the organic solvent from the mixed solution.
<80> The method according to any one of <68> to <79> for stabilizing the pharmaceutical composition according to any one of <1> to <55>, wherein the method for stabilizing the pharmaceutical composition is a method of suppressing the change in the average particle size of the lipid complex in the pharmaceutical composition.
<81> The method according to <80> for stabilizing the pharmaceutical composition according to any one of <1> to <55>, wherein the method of suppressing the change in the average particle size is a method of suppressing increase in the average particle size.

### Advantageous Effects of Invention

In accordance with the present invention, a novel pharmaceutical composition comprising a double-stranded ribonucleic acid capable of suppressing expression of complement C5, a method for producing the pharmaceutical composition, and a method for stabilizing the pharmaceutical composition can be provided.

The pharmaceutical composition of the present invention can suppress expression of complement C5 to suppress hemolysis, and hence can be applicable as a therapeutic agent for paroxysmal nocturnal hemoglobinuria (PNH) and atypical hemolytic uremic syndrome (aHUS).

### BRIEF DESCRIPTION OF DRAWINGS

FIG.1 shows graphs representing results of liver C5 mRNA residual rates after administration of siRNA-008 and liver C5 mRNA residual rates after administration of siRNA-008-34 in Example 5.

FIG. 1 shows graphs representing results of plasma C5 residual rates after administration of siRNA-008 and plasma C5 residual rates after administration of siRNA-008-34 in Example 5.

FIG. 2 shows a graph representing results of complement activity after administration of siRNA-008-34 in Example 8.

FIG. 3 shows a graph representing results of complement activity after administration of siRNA-008-34 in Example 9.

FIG. 4 shows a graph representing results of complement activity after administration of siRNA-008-34 in Example 10.

FIG. 5 shows a graph representing results of complement activity after administration of siRNA-008-34 in Example 11.

### DESCRIPTION OF EMBODIMENTS

Examples of genes encoding complement C5 targeted by the double-stranded ribonucleic acid of an embodiment include, but are not limited to, C5 derived from humans, mice, and monkeys. Information on C5 gene sequences is available from public databases including registered sequence information such as GenBank provided by The National Center for Biotechnology Information (NCBI), or can be obtained by designing a primer based on information of a nucleotide sequence for C5 from a closely related animal species followed by cloning therewith from an RNA extracted from a desired animal species. Examples of the sequence of an mRNA transcript corresponding to the target gene human C5 include the sequence of a human C5 mRNA transcript registered as GenBank Accession No. NM_001735.2 (GI: 38016946). The term “C5 gene” herein is not limited to a gene having a particular sequence. For example, naturally-occurring C5 genes with single nucleotide polymorphism can be also included in the term.

In the double-stranded ribonucleic acid comprising a sense strand and an antisense strand in an embodiment, the combination of the sense strand and the antisense strand is selected from the group consisting of a sense strand consisting of a nucleotide sequence set forth in SEQ ID NO: 13 and an antisense strand consisting of a nucleotide sequence set forth in SEQ ID NO: 14, a sense strand consisting of a nucleotide sequence set forth in SEQ ID NO: 159 and an antisense strand consisting of a nucleotide sequence set forth in SEQ ID NO: 160, a sense strand consisting of a nucleotide sequence set forth in SEQ ID NO: 115 and an antisense strand consisting of a nucleotide sequence set forth in SEQ ID NO: 116, a sense strand consisting of a nucleotide sequence set forth in SEQ ID NO: 117 and an antisense strand consisting of a nucleotide sequence set forth in SEQ ID NO: 118, a sense strand consisting of a nucleotide sequence set forth in SEQ ID NO: 119 and an antisense strand consisting of a nucleotide sequence set forth in SEQ ID NO: 120, a sense strand consisting of a nucleotide sequence set forth in SEQ ID NO: 121 and an antisense strand consisting of a nucleotide sequence set forth in SEQ ID NO: 122, a sense strand consisting of a nucleotide sequence set forth in SEQ ID NO: 123 and an antisense strand consisting of a nucleotide sequence set forth in SEQ ID NO: 124, a sense strand consisting of a nucleotide sequence set forth in SEQ ID NO: 125 and an antisense strand consisting of a nucleotide sequence set forth in SEQ ID NO: 126, a sense strand consisting of a nucleotide sequence set forth in SEQ ID NO: 127 and an antisense strand consisting of a nucleotide sequence set forth in SEQ ID NO: 128, a sense strand consisting of a nucleotide sequence set forth in SEQ ID NO: 129 and an antisense strand consisting of a nucleotide sequence set forth in SEQ ID NO: 130, a sense strand consisting of a nucleotide sequence set forth in SEQ ID NO: 131 and an antisense strand consisting of a nucleotide sequence set forth in SEQ ID NO: 132, a sense strand consisting of a nucleotide sequence set forth in SEQ ID NO: 133 and an antisense strand consisting of a nucleotide sequence set forth in SEQ ID NO: 134, a sense strand consisting of a nucleotide sequence set forth in SEQ ID NO: 137 and an antisense strand consisting of a nucleotide sequence set forth in SEQ ID NO: 138, a sense strand consisting of a nucleotide sequence set forth in SEQ ID NO: 139 and an antisense strand consisting of a nucleotide sequence set forth in SEQ ID NO: 140, a sense strand consisting of a nucleotide sequence set forth in SEQ ID NO: 141 and an antisense strand consisting of a nucleotide sequence set forth in SEQ ID NO: 142, a sense strand consisting of a nucleotide sequence set forth in SEQ ID NO: 143 and an antisense strand consisting of a nucleotide sequence set forth in SEQ ID NO: 144, a sense strand consisting of a nucleotide sequence set forth in SEQ ID NO: 145 and an antisense strand consisting of a nucleotide sequence set forth in SEQ ID NO: 146, a sense strand consisting of a nucleotide sequence set forth in SEQ ID NO: 147 and an antisense strand consisting of a nucleotide sequence set forth in SEQ ID NO: 148, a sense strand consisting of a nucleotide sequence set forth in SEQ ID NO: 149 and an antisense strand consisting of a nucleotide sequence set forth in SEQ ID NO: 150, a sense strand consisting of a nucleotide sequence set forth in SEQ ID NO: 151 and an antisense strand consisting of a nucleotide sequence set forth in SEQ ID NO: 152, and a sense strand consisting of a nucleotide sequence set forth in SEQ ID NO: 153 and an antisense strand consisting of a nucleotide sequence set forth in SEQ ID NO: 154. The combinations respectively correspond to the sequences of siRNA-008, siRNA-008-01, siRNA-008-02, siRNA-008-08, siRNA-008-09, siRNA-008-10, siRNA-008- 11, siRNA-008- 12, siRNA-008- 13, siRNA-008- 14, siRNA-008-22, siRNA-008-23, siRNA-008-30, siRNA-008-31, siRNA-008-32, siRNA-008-33, siRNA-008-34, siRNA-008-35, siRNA-008-36, siRNA-008-37, and siRNA-008-38 in the present specification.

In the double-stranded ribonucleic acid in the embodiment, a sense strand and an antisense strand as any one of the combinations (1) to (21) are pairing.
(1) a sense strand consisting of a nucleotide sequence set forth in SEQ ID NO: 13, and an antisense strand consisting of a nucleotide sequence set forth in SEQ ID NO: 14
(2) a sense strand consisting of a nucleotide sequence set forth in SEQ ID NO: 159, and an antisense strand consisting of a nucleotide sequence set forth in SEQ ID NO: 160
(3) a sense strand consisting of a nucleotide sequence set forth in SEQ ID NO: 115, and an antisense strand consisting of a nucleotide sequence set forth in SEQ ID NO: 116
(4) a sense strand consisting of a nucleotide sequence set forth in SEQ ID NO: 117, and an antisense strand consisting of a nucleotide sequence set forth in SEQ ID NO: 118
(5) a sense strand consisting of a nucleotide sequence set forth in SEQ ID NO: 119, and an antisense strand consisting of a nucleotide sequence set forth in SEQ ID NO: 120
(6) a sense strand consisting of a nucleotide sequence set forth in SEQ ID NO: 121, and an antisense strand consisting of a nucleotide sequence set forth in SEQ ID NO: 122
(7) a sense strand consisting of a nucleotide sequence set forth in SEQ ID NO: 123, and an antisense strand consisting of a nucleotide sequence set forth in SEQ ID NO: 124
(8) a sense strand consisting of a nucleotide sequence set forth in SEQ ID NO: 125, and an antisense strand consisting of a nucleotide sequence set forth in SEQ ID NO: 126
(9) a sense strand consisting of a nucleotide sequence set forth in SEQ ID NO: 127, and an antisense strand consisting of a nucleotide sequence set forth in SEQ ID NO: 128
(10) a sense strand consisting of a nucleotide sequence set forth in SEQ ID NO: 129, and an antisense strand consisting of a nucleotide sequence set forth in SEQ ID NO: 130
(11) a sense strand consisting of a nucleotide sequence set forth in SEQ ID NO: 131, and an antisense strand consisting of a nucleotide sequence set forth in SEQ ID NO: 132
(12) a sense strand consisting of a nucleotide sequence set forth in SEQ ID NO: 133, and an antisense strand consisting of a nucleotide sequence set forth in SEQ ID NO: 134
(13) a sense strand consisting of a nucleotide sequence set forth in SEQ ID NO: 137, and an antisense strand consisting of a nucleotide sequence set forth in SEQ ID NO: 138
(14) a sense strand consisting of a nucleotide sequence set forth in SEQ ID NO: 139, and an antisense strand consisting of a nucleotide sequence set forth in SEQ ID NO: 140
(15) a sense strand consisting of a nucleotide sequence set forth in SEQ ID NO: 141, and an antisense strand consisting of a nucleotide sequence set forth in SEQ ID NO: 142
(16) a sense strand consisting of a nucleotide sequence set forth in SEQ ID NO: 143, and an antisense strand consisting of a nucleotide sequence set forth in SEQ ID NO: 144
(17) a sense strand consisting of a nucleotide sequence set forth in SEQ ID NO: 145, and an antisense strand consisting of a nucleotide sequence set forth in SEQ ID NO: 146
(18) a sense strand consisting of a nucleotide sequence set forth in SEQ ID NO: 147, and an antisense strand consisting of a nucleotide sequence set forth in SEQ ID NO: 148
(19) a sense strand consisting of a nucleotide sequence set forth in SEQ ID NO: 149, and an antisense strand consisting of a nucleotide sequence set forth in SEQ ID NO: 150
(20) a sense strand consisting of a nucleotide sequence set forth in SEQ ID NO: 151, and an antisense strand consisting of a nucleotide sequence set forth in SEQ ID NO: 152
(21) a sense strand consisting of a nucleotide sequence set forth in SEQ ID NO: 153, and an antisense strand consisting of a nucleotide sequence set forth in SEQ ID NO: 154

Each of the combinations (1) to (21) of a sense strand and an antisense strand includes a region complementary to each other. For example, a double-stranded ribonucleic acid including the combination (1) of the sense strand set forth in SEQ ID NO: 13 and the antisense strand set forth in SEQ ID NO: 14 includes the following complementary strands (dT\hat{ }dT at the 3′-terminal is not shown, see Table 1 for more details).

(SEQ ID NO: 13)
5′-uGGuAuAuGuGuuGcuGAu-3′
(SEQ ID NO: 14)
3′-AcCAuAuAcAcAAcGAcUA-5′

In the double-stranded ribonucleic acid comprising a sense strand and an antisense strand in an embodiment, the combination of the sense strand and the antisense strand is selected from the group consisting of a sense strand consisting of a nucleotide sequence set forth in SEQ ID NO: 159 and an antisense strand consisting of a nucleotide sequence set forth in SEQ ID NO: 160, a sense strand consisting of a nucleotide sequence set forth in SEQ ID NO: 139 and an antisense strand consisting of a nucleotide sequence set forth in SEQ ID NO: 140, a sense strand consisting of a nucleotide sequence set forth in SEQ ID NO: 141 and an antisense strand consisting of a nucleotide sequence set forth in SEQ ID NO: 142, a sense strand consisting of a nucleotide sequence set forth in SEQ ID NO: 143 and an antisense strand consisting of a nucleotide sequence set forth in SEQ ID NO: 144, a sense strand consisting of a nucleotide sequence set forth in SEQ ID NO: 145 and an antisense strand consisting of a nucleotide sequence set forth in SEQ ID NO: 146, a sense strand consisting of a nucleotide sequence set forth in SEQ ID NO: 147 and an antisense strand consisting of a nucleotide sequence set forth in SEQ ID NO: 148, and a sense strand consisting of a nucleotide sequence set forth in SEQ ID NO: 153 and an antisense strand consisting of a nucleotide sequence set forth in SEQ ID NO: 154. The combinations respectively correspond to the sequences of siRNA-008-01, siRNA-008-31, siRNA-008-32, siRNA-008-33, siRNA-008-34, siRNA-008-35, and siRNA-008-38 in the present specification.

In the double-stranded ribonucleic acid comprising a sense strand and an antisense strand in an embodiment, the combination of the sense strand and the antisense strand is selected from the group consisting of a sense strand consisting of a nucleotide sequence set forth in SEQ ID NO: 159 and an antisense strand consisting of a nucleotide sequence set forth in SEQ ID NO: 160, a sense strand consisting of a nucleotide sequence set forth in SEQ ID NO: 141 and an antisense strand consisting of a nucleotide sequence set forth in SEQ ID NO: 142, a sense strand consisting of a nucleotide sequence set forth in SEQ ID NO: 143 and an antisense strand consisting of a nucleotide sequence set forth in SEQ ID NO: 144, a sense strand consisting of a nucleotide sequence set forth in SEQ ID NO: 145 and an antisense strand consisting of a nucleotide sequence set forth in SEQ ID NO: 146, a sense strand consisting of a nucleotide sequence set forth in SEQ ID NO: 147 and an antisense strand consisting of a nucleotide sequence set forth in SEQ ID NO: 148, and a sense strand consisting of a nucleotide sequence set forth in SEQ ID NO: 153 and an antisense strand consisting of a nucleotide sequence set forth in SEQ ID NO: 154. The combinations respectively correspond to the sequences of siRNA-008-01, siRNA-008-32, siRNA-008-33, siRNA-008-34, siRNA-008-35, and siRNA-008-38 in the present specification.

In the double-stranded ribonucleic acid comprising a sense strand and an antisense strand in an embodiment, the combination of the sense strand and the antisense strand is selected from the group consisting of a sense strand consisting of a nucleotide sequence set forth in SEQ ID NO: 141 and an antisense strand consisting of a nucleotide sequence set forth in SEQ ID NO: 142, a sense strand consisting of a nucleotide sequence set forth in SEQ ID NO: 143 and an antisense strand consisting of a nucleotide sequence set forth in SEQ ID NO: 144, a sense strand consisting of a nucleotide sequence set forth in SEQ ID NO: 145 and an antisense strand consisting of a nucleotide sequence set forth in SEQ ID NO: 146, a sense strand consisting of a nucleotide sequence set forth in SEQ ID NO: 147 and an antisense strand consisting of a nucleotide sequence set forth in SEQ ID NO: 148, and a sense strand consisting of a nucleotide sequence set forth in SEQ ID NO: 153 and an antisense strand consisting of a nucleotide sequence set forth in SEQ ID NO: 154. The combinations respectively correspond to the sequences of siRNA-008-32, siRNA-008-33, siRNA-008-34, siRNA-008-35, and siRNA-008-38 in the present specification.

In an embodiment, the double-stranded ribonucleic acid comprising a sense strand and an antisense strand comprises a sense strand consisting of a nucleotide sequence set forth in SEQ ID NO: 159, and an antisense strand consisting of a nucleotide sequence set forth in SEQ ID NO: 160. The combination corresponds to the sequence of siRNA-008-01 in the present specification.

In an embodiment, the double-stranded ribonucleic acid comprising a sense strand and an antisense strand comprises a sense strand consisting of a nucleotide sequence set forth in SEQ ID NO: 141, and an antisense strand consisting of a nucleotide sequence set forth in SEQ ID NO: 142. The combination corresponds to the sequence of siRNA-008-32 in the present specification.

In an embodiment, the double-stranded ribonucleic acid comprising a sense strand and an antisense strand comprises a sense strand consisting of a nucleotide sequence set forth in SEQ ID NO: 143, and an antisense strand consisting of a nucleotide sequence set forth in SEQ ID NO: 144. The combination corresponds to the sequence of siRNA-008-33 in the present specification.

In an embodiment, the double-stranded ribonucleic acid comprising a sense strand and an antisense strand comprises a sense strand consisting of a nucleotide sequence set forth in SEQ ID NO: 145, and an antisense strand consisting of a nucleotide sequence set forth in SEQ ID NO: 146. The combination corresponds to the sequence of siRNA-008-34 in the present specification.

In an embodiment, the double-stranded ribonucleic acid comprising a sense strand and an antisense strand comprises a sense strand consisting of a nucleotide sequence set forth in SEQ ID NO: 147, and an antisense strand consisting of a nucleotide sequence set forth in SEQ ID NO: 148. The combination corresponds to the sequence of siRNA-008-35 in the present specification.

In an embodiment, the double-stranded ribonucleic acid comprising a sense strand and an antisense strand comprises a sense strand consisting of a nucleotide sequence set forth in SEQ ID NO: 153, and an antisense strand consisting of a nucleotide sequence set forth in SEQ ID NO: 154. The combination corresponds to the sequence of siRNA-008-38 in the present specification.

The antisense strand in an embodiment is substantially complementary to at least a part of an mRNA transcript of a C5 gene. Here, the phrase “substantially complementary” includes not only cases that the antisense strand is completely complement to a part of an mRNA transcript of a C5 gene but also cases that there are one to several acceptable mismatches between the antisense strand and a part of an mRNA transcript of C5 gene.

The sense strand in an embodiment is substantially complementary to at least a part of the nucleotide sequence of the antisense strand. The phrase “substantially complementary” includes not only cases that the sense strand is completely complement to a part of the nucleotide sequence of the antisense strand but also cases that there are one to several acceptable mismatches between the sense strand and a part of the nucleotide sequence of the antisense strand. The phrase “completely complementary” may apply to cases when the oligonucleotide of the longer of the sense strand and the antisense strand includes a nucleotide sequence completely complementary to the oligonucleotide of the shorter.

The double-stranded ribonucleic acid in an embodiment also includes a modified nucleotide, as described later (see also Table 1). Hence, the term “nucleotide” used herein is intended not only to refer to guanosine-3′-phosphate, cytidine-3′-phosphate, adenosine-3′-phosphate, and uridine-3′-phosphate, but also to encompass various modified nucleotides.

The term “double-stranded ribonucleic acid” or “dsRNA” herein refers to a ribonucleic acid (RNA) molecule having double-stranded structure including two antiparallel, substantially complementary oligonucleotides, or a complex thereof Examples of double-stranded ribonucleic acids herein include, but are not limited to, siRNAs (small interfering RNAs). The double-stranded ribonucleic acid of an embodiment comprises a sense strand and an antisense strand. Through RNAi using the double-stranded ribonucleic acid of an embodiment, an mRNA for a C5 gene is cleaved as the target mRNA molecule in an RISC complex, and as a result expression of C5 is suppressed. For example, expression of C5 in cells in a subject is suppressed.

The double-stranded ribonucleic acid of an embodiment can be synthesized, for example, by using a method with chemical synthesis known in the art (e.g., described in Nucleic Acid Research, 35(10), 3287-96 (2007)) and enzymatic transcription.

The double-stranded ribonucleic acid of an embodiment includes various modifications. Modification can be performed by using a method known in the art. Examples of the modification include sugar modification.

Examples of the sugar modification include modification for the ribose moiety constituting ribonucleoside, specifically, substitution or addition at the hydroxy group at the 2′-position, more specifically, 2′-O-methyl-modified nucleotide in which the hydroxy group has been substituted with a methoxy group. Nucleotides represented as lowercase a, u, g, and c in Table 1 are 2′-O-methyl-modified nucleotides, and the sense strand and antisense strand of the double-stranded ribonucleic acid of an embodiment each include 2′-O-methyl-modified nucleotide.

The double-stranded ribonucleic acid can be modified by inserting an additional nucleotide or nucleotide derivative, which is called overhang, into the 3′-side or 5′-side of a region where the sense strand and the antisense strand are forming a double strand. The double-stranded ribonucleic acid of an embodiment includes the sense strand and/or the antisense strand including deoxy-thymidine (dT) at the 3′-terminal as SEQ ID NO: 13 and SEQ ID NO: 14, and the sense strand and the antisense strand including inverted deoxy-thymidine (idT) as SEQ ID NO: 129. The double-stranded ribonucleic acid of an embodiment also includes the sense strand and/or the antisense strand including U, A, and so forth, added as an overhang sequence, for example, that including UUUU added at the 3′-terminal of the antisense strand as SEQ ID NOs: 140 and 142.

Alternatively, the double-stranded ribonucleic acid can be backbone-modified through modification or substitution of the phosphodiester bond. Examples of the modification or substitution of the phosphodiester bond include a phosphorothioate bond. The double-stranded ribonucleic acid of an embodiment also includes that including neighboring nucleotides connected with a phosphorothioate bond as SEQ ID NO: 13, SEQ ID NO: 14, and SEQ ID NO: 121.

### (Lipid Complex)

The pharmaceutical composition of the present embodiment comprises a lipid complex comprising a double-stranded ribonucleic acid. In an embodiment, the lipid complex comprises (I) the above double-stranded ribonucleic acid, (II) a cationic lipid, and (III) at least one lipid selected from the group consisting of neutral lipid, polyethylene glycol-modified lipid (PEG lipid), and sterol. Examples of the lipid complex herein include, but are not limited to, LNPs (lipid nanoparticles). In a particular embodiment, the pharmaceutical composition comprises a lipid complex encapsulating a double-stranded ribonucleic acid. The pharmaceutical composition of another embodiment comprises a lipid nanoparticle comprising a double-stranded ribonucleic acid.

Examples of the form of a lipid complex formed of a lipid containing a cationic lipid and the double-stranded ribonucleic acid include a complex of the double-stranded ribonucleic acid and a membrane consisting of a lipid monolayer (single molecule) (reverse micelle); a complex of the double-stranded ribonucleic acid and a liposome; and a complex of the double-stranded ribonucleic acid and a micelle. In a lipid complex of an embodiment of the present invention, the double-stranded ribonucleic acid is encapsulated in a fine particle comprising a lipid containing a cationic lipid.

In an embodiment, the lipid complex contains the double-stranded ribonucleic acid in a content of, for example, 0.01 to 50% by weight, 0.1 to 30% by weight, or 1 to 10% by weight to the total weight of the lipid complex.

Cationic lipid is an amphiphilic molecule having a lipophilic region including one or more hydrocarbon groups and a hydrophilic region including a polar group to be protonated at specific pH. Examples of the cationic lipid of an embodiment include, but are not particularly limited to, cationic lipids described in International Publication Nos. WO 2015/105131, WO 2016/104580, and WO 2017/222016, and alternatively a cationic lipid with improved biodegradability described in International Publication No. WO 2016/104580 or WO 2017/222016 can be used. Examples of the cationic lipid of an embodiment include 1-oxo-1-(undecan-5-yloxy)nonadecan-10-yl-1-methylpiperidine-4-carboxylate, 1-((2-butyloctyl)oxy)-1-oxononadecan-10-yl-1-methylpiperidine-4-carboxylate, 1-oxo-1-(undecan-5-yloxy)heptadecan-8-yl-1-methylpip eri dine 4-carboxylate, 21-oxo-21-(undecan-5-yloxy)heneicosan-10-yl-1-methylpiperidine4-carboxylate, 21-(octan-3-yloxy)-21-oxoheneicosan-10-yl-1-methylpiperidine-4-carboxylate, 1-((2-butylo ctyl)oxy)-1-oxoicosan-10-yl-1-methylpiperidine-4-carboxylate, (Z)-1-((2-butylnon-3-en-1-yl) oxy)-1-oxoicosan-10-yl-1-methylpiperidine-4-carboxylate, 1-oxo-1-((3-pentyloctyl)oxy)icosan-10-yl-1-methylpiperidine-4-carboxylate, 1-((3,4-dipropylheptyl)oxy)-1-oxoicosan-10-yl-1-methylpiperidine-4-carboxylate, 14(6- (butyldisulfanyl)-3-(3-(butyldisulfanyl)propyl)hexyl)oxy)-1-oxoicosan-10-y1-1-methylpiperidine-4-carboxylate, 2-butyloctyl-10-((4-(dimethylamino)butanoyl)oxy)icosanoate, 2-{9-[(2-butyloctyl)oxy]-9-oxononyl}dodecyl 1-methylpiperidine-4-carboxylate, 2-{9-oxo-9-[(3-pentyloctyl)oxy]nonyl}dodecyl 1-methylpiperidine-4-carboxylate, 2-nonyl-11- oxo-11-[(3-pentyloctyl)oxy]undecyl 1-methylpiperidine-4-carboxylate, bis(3-pentyloctyl) 9-{[1(1-methylpiperidine-4-carbonyl)oxy]methyl} heptadecanedioate, di[(Z)-2-nonen-l-yl]9-{1[(1-methylpiperidine-4-carbonyl)oxy]methyl}heptadecanedioate, 1-(2-octylcyclopropyl)heptadecan-8-yl-1-methylpiperidine-4-carboxylate, (3 S)-2-{9-oxo-9-[(3-pentyloctypoxy]nonyl}dodecyl 1-methylpyrrolidine-3-carboxylate, and (3R)-2-{9-oxo-9-[(3-pentyloctypoxy]nonyl}dodecyl 1-methylpyrrolidine-3-carboxylate. In an embodiment, the cationic lipid is 1((2-butyloctyl)oxy)-1-oxoicosan-10-yl-1-methylpiperidine-4-carboxylate, 1-((2-butyloctyl)oxy)-1-oxononadecan-10-yl-1-methylpiperidine-4-carboxylate, 2-{9-oxo-9-[(3-pentyloctyl)oxy]nonyl}dodecyl 1-methylpiperidine-4-carboxylate, 1-(2-octylcyclopropyl)heptadecan-8-yl-1-methylpiperidine-4-carboxylate, (3 S)-2-{9-oxo-9-[(3-pentyloctypoxy]nonyl}dodecyl 1-methylpyrroli dine-3-carboxylate, or (3R)-2-{9-oxo-9-[(3-pentyloctypoxy]nonyl}dodecyl 1-methylpyrrolidine-3-carboxylate. In a particular embodiment, the cationic lipid is 2-{9-oxo-9-[(3-pentyl octyl) oxy]nonyl}dodecyl 1-methylpiperidine-4-carboxylate.

The lipid complex in an embodiment contains the above-described cationic lipid in a content of, for example, 10 to 100 mol %, 20 to 90 mol %, 30 to 90 mol %, or 40 to 70 mol % based on the total lipids contained in the lipid complex. One cationic lipid can be used singly, and mixture of two or more cationic lipids can also be used.

The lipid complex in an embodiment comprises (I) the above-described cationic lipid and (II) at least one lipid selected from the group consisting of neutral lipid, polyethylene glycol-modified lipid, and sterol, as a lipid component. The lipid complex of an embodiment contains the lipid component in a content of, for example, 50 to 99.99% by weight, 70 to 99.9% by weight, or 90 to 99% by weight to the total weight of the lipid complex.

The term “neutral lipid” refers to a lipid present either as a non-charged form or as a neutral zwitterion at physiological pH. Examples of the neutral lipid of the present embodiment include phospholipid and ceramide. Examples of the phospholipid of the present embodiment include DOPE (1,2-Dioleoyl-sn-glycero-3-phosphoethanol amine), POPE (1-Palmitoyl-2-oleoyl-sn-glycero-3-phosphoethanolamine), HSPC (Hydrogenated soybean phosphatidylcholine), SOPC (1-Stearoyl-2-oleoyl-sn-glycero-3-phosphocholine), POPC (1-Palmitoyl-2-oleoyl- sn-glycero-3-phosphocholine), EPC (Egg phosphatidylcholine), DMPC (1,2-Dimyri stoyl-sn-glycero-3-pho sphocholine), DPPC (1,2-Dip almitoyl-sn-glycero-3-phosphocholine), DSPC (1,2-Distearoyl-sn-glycero-3-phosphocholine), DAPC (1,2-Diarachidoyl-sn-glycero-3-phosphocholine), DBPC (1,2-Dibehenoyl-sn-glycero-3-phosphocholine), DLPC (1,2-Dilauroyl-sn-glycero-3-phosphocholine), DOPC (1,2-Dioleoyl-sn-glycero-3-phosphocholine), DOPG (1,2-Dioleoyl-sn-glycero-3-phosphoglycerol), DPPG (1,2-Dip almitoyl-sn-glycero-3-phosphoglycerol), DSPG (1,2-Di stearoyl-sn-glycero-3-phosphoglycerol), DOPS (1,2-Dioleoyl-sn-glycero-3-phospho-L-serine), DOPE-MAL (N-(3-Maleimide-l-oxopropyl)-L-α-phosphatidylethanolamine, Dioleoyl), and sphingomyelin. In an embodiment, the neutral lipid is DOPE, HSPC, DPPC, DSPC, or DAPC. In a particular embodiment, the neutral lipid is DSPC. One neutral lipid can be used singly, and mixture of two or more neutral lipids can also be used.

The lipid complex in an embodiment may contain the neutral lipid in a content of, for example, 0 to 50 mol %, 0 to 40 mol %, 0 to 30 mol %, or 0 to 20 mol % based on the total lipids contained in the lipid complex. In another embodiment, the lipid complex may contain the neutral lipid in a content of, for example, 0.1 to 20 mol % or 3 to 15 mol % based on the total lipids contained in the lipid complex.

The polyethylene glycol-modified lipid (PEG lipid) refers to a lipid having a polyethylene glycol group. Examples of the polyethylene glycol-modified lipid (PEG lipid) in an embodiment include PEG2000-DMG PEG2000-DPG PEG2000-DSG PEG5000-DMG PEG5000-DPG PEG5000-DSG PEG-cDMA, PEG-C-DOMG PEG-DAG PEG-DAA, PEG-phospholipid, PEG-cholesterol, and PEG-ceramide (Cer). Examples of PEG2000-DMG PEG2000-DPG PEG2000-DSG PEG-cDMA, and PEG-C-DOMG. In a particular embodiment, the polyethylene glycol-modified lipid is PEG2000-DMG. The PEG in the present specification also includes methoxy PEG (MPEG). Specifically, for example, MPEG2000-DMG is included in PEG2000-DMG and MPEG2000-DPG is also included in PEG2000-DPG One polyethylene glycol-modified lipid can be used singly, and mixture of two or more polyethylene glycol-modified lipids can also be used.

The lipid complex in an embodiment may contain the polyethylene glycol-modified lipid in a content of, for example, 0 to 30 mol %, 0 to 20 mol %, 0 to 10 mol %, or 0.5 to 2 mol % based on the total lipids contained in the lipid complex. In another embodiment, the lipid complex may contain the polyethylene glycol-modified lipid in a content of, for example, 0.01 to 10 mol % or 0.1 to 3 mol % based on the total lipids contained in the lipid complex.

Sterol is an alcohol having a steroid backbone. Examples of the sterol of an embodiment include cholesterol, dihydrocholesterol, lanosterol, β-sitosterol, campesterol, stigmasterol, brassicasterol, ergosterol, fucosterol, and 3β-[N-(N′,N′-dimethylaminoethyl)carbamoyl]cholesterol (DC-Chol). In an embodiment, the sterol is cholesterol, dihydrocholesterol, lanosterol, or β-sitosterol. In a particular embodiment, the sterol is cholesterol. One sterol can be used singly, and mixture of two or more sterols can also be used.

The lipid complex in an embodiment may contain the sterol in a content of, for example, 0 to 90 mol %, 10 to 80 mol %, or 20 to 40 mol % based on the total lipids contained in the lipid complex. In another embodiment, the lipid complex may contain the sterol in a content of, for example, 0.1 to 70 mol % or 15 to 60 mol % based on the total lipids contained in the lipid complex.

Combination of lipid components in the lipid complex in an embodiment is not particularly limited, and examples thereof include combination of the above-described cationic lipid, neutral lipid, and sterol, and combination of the above-described cationic lipid, neutral lipid, polyethylene glycol-modified lipid, and sterol.

In an embodiment, the lipid complex comprises a cationic lipid, neutral lipid, polyethylene glycol-modified lipid, and sterol. The cationic lipid is needed for encapsulation of a nucleic acid or efficient delivery of a nucleic acid to target cells, and the polyethylene glycol-modified lipid has been reported to be needed for preventing particles from aggregating (Molecular Therapy-Nucleic Acids (2012)1, e37). Moreover, it has been reported that coexistence of four lipids: neutral lipid and sterol in addition to those two lipids, is crucial for encapsulation of a nucleic acid to form stable particles (Nanoscale. 2019 Nov. 21; 11(45): 21733-21739.).

The lipid complex in an embodiment may be a lipid complex containing, for example, 2-{9-oxo-9-[(3-pentyloctyl)oxy]nonyl}dodecyl 1-methylpiperidine-4-carboxylate and at least one lipid selected from the group consisting of DSPC, PEG2000-DMQ and cholesterol, or a lipid complex containing 2-{9-oxo-9-[(3-pentyloctyl)oxy]nonyl}dodecyl 1-methylpiperidine-4-carboxylate, DSPC, PEG2000-DMQ and cholesterol.

The lipid complex in an embodiment encapsulating the double-stranded ribonucleic acid comprises lipid components of cationic lipid/neutral lipid/polyethylene glycol-modified lipid/sterol, and the mole ratio of the lipids may be, for example, 10 to 99/0 to 50/0 to 10/0 to 50, 10 to 99/1 to 50/0.5 to 10/10 to 50, 40 to 70/1 to 20/0.5 to 2/20 to 40, or 40 to 70/0 to 20/0.5 to 2/20 to 40. In an embodiment, the mole ratio of cationic lipid/neutral lipid/polyethylene glycol-modified lipid/sterol in the lipid complex is 30 to 90/0.1 to 20/0.01 to 10/0.1 to 70. In another embodiment, the mole ratio of cationic lipid/neutral lipid/polyethylene glycol-modified lipid/sterol in the lipid complex is 40 to 70/3 to 15/0.1 to 3/15 to 60. In a particular embodiment, the mole ratio of cationic lipid/neutral lipid/polyethylene glycol-modified lipid/sterol in the lipid complex is 60/10.5/1.5/28.

The “average particle size” of the lipid complex in the present specification refers to the Z-average particle size, and the average particle size is measured by a dynamic light scattering method. The average particle size (Z-average) of the lipid complex comprising a double-stranded ribonucleic acid may be, for example, 10 to 1000 nm, 30 to 500 nm, or 30 to 200 nm, though the average particle size is not particularly limited thereto. In an embodiment, the average particle size of the lipid complex comprising a double-stranded ribonucleic acid is 100 nm or less. In a particular embodiment, the average particle size of the lipid complex comprising a double-stranded ribonucleic acid is 65 nm or more and 100 nm or less. In another embodiment, the average particle size of the lipid complex comprising a double-stranded ribonucleic acid is 80 nm or more and 100 nm or less, and, in still another embodiment, the average particle size of the lipid complex comprising a double-stranded ribonucleic acid is 85 nm or more and 100 nm or less.

In an embodiment, the pH of a solution of the lipid complex is 5.0 or less or 7.5 or more. In another embodiment, the pH of a solution of the lipid complex is 2.0 or more and 5.0 or less, or 7.5 or more and 11.0 or less. In another embodiment, the pH of a solution of the lipid complex is 2.0 or more and 5.0 or less, 2.5 or more and 5.0 or less, 3.0 or more and 5.0 or less, 3.5 or more and 5.0 or less, 4.0 or more and 5.0 or less, 4.5 or more and 5.0 or less, 7.5 or more and 11.0 or less, 7.5 or more and 10.5 or less, 7.5 or more and 10.0 or less, 7.5 or more and 9.5 or less, 7.5 or more and 9.0 or less, or 7.5 or more and 8.5 or less. In a particular embodiment, the pH of a solution of the lipid complex is 7.5 or more and 8.5 or less. The storage stability of the pharmaceutical composition is improved by setting the pH of a solution of the lipid complex in the above range.

The storage stability of the pharmaceutical composition of the present embodiment can be determined, for example, on the basis of the degree of change in the average particle size by comparing the average particle size of the lipid complex before storage and the average particle size of the lipid complex after storage for a given period of time. In the present specification, “before storage” may be, for example, the time immediately after production of the pharmaceutical composition, or, if the pH has been adjusted, the time immediately after the pH adjustment. Examples of conditions for storage of the pharmaceutical composition of the present embodiment include conditions that allow to keep in a cool place or refrigerator (conditions for refrigerated storage), and normal temperature or room temperature. In an embodiment, a condition for the storage of the pharmaceutical composition is 2 to 8° C. In another embodiment, a condition for the storage of the pharmaceutical composition is 5° C. In still another embodiment, a condition for the storage of the pharmaceutical composition is 25° C.

The storage stability of the pharmaceutical composition may be determined, for example, on the basis of the average particle size of the lipid complex 2 weeks after the initiation of storage (after storage for 2 weeks), or determined on the basis of the average particle size of the lipid complex 1 month after the initiation of storage (after storage for 1 month), or determined on the basis of the average particle size of the lipid complex 2 months after the initiation of storage (after storage for 2 months), or determined on the basis of the average particle size of the lipid complex 3 months after the initiation of storage (after storage for 3 months). In an embodiment, the storage stability may be determined to be good if the change in the average particle size of the lipid complex after storage is ±10% or less from the average particle size of the lipid complex before storage, the storage stability may be determined to be good if the change in the average particle size of the lipid complex after storage is ±8% or less from the average particle size of the lipid complex before storage, and the storage stability may be determined to be good if the change in the average particle size of the lipid complex after storage is ±5% or less from the average particle size of the lipid complex before storage. Specifically, for example, the storage stability may be determined to be good if the average particle size of the lipid complex 2 weeks after the initiation of storage differs by ±10% or less from the average particle size of the lipid complex before the storage. In another embodiment, the storage stability of the pharmaceutical composition can be determined on the basis of increase in the average particle size, and, for example, the storage stability may be determined to be good if the average particle size of the lipid complex after storage for a given period of time differs by +10% or less from the average particle size of the lipid complex before the storage, the storage stability may be determined to be good if the average particle size of the lipid complex after storage for a given period of time differs by +8% or less from the average particle size of the lipid complex before the storage, and the storage stability may be determined to be good if the average particle size of the lipid complex after storage for a given period of time differs by +5% or less from the average particle size of the lipid complex before the storage. Specifically, for example, the storage stability of the pharmaceutical composition may be determined to be good if the average particle size of the lipid complex 2 weeks after the initiation of storage differs by +10% or less from the average particle size of the lipid complex before the storage.

The siRNA encapsulation efficiency for a lipid complex in an embodiment encapsulating the double-stranded ribonucleic acid can be calculated, for example, from the siRNA concentration of a formulation diluted with RNase Free Water, which is assumed as the concentration of siRNA present in the LNP external solution, and the siRNA concentration of the formulation diluted with 1% Triton X-100, which is assumed as the total siRNA concentration of the formulation, where each siRNA concentration is measured by using Quant-iT RiboGreen RNA Reagent (Invitrogen, Cat#R11491) (see also Kewal K. Jain, Drug Delivery System, Methods in Molecular Biology, Vol. 1141: 109-120). The encapsulation efficiency calculated in this manner is, for example, higher than 80%, higher than 85%, or higher than 90%. The siRNA encapsulation efficiency for a lipid complex in an embodiment is higher than 90%.

### <Method for Producing Lipid Complex>

Examples of methods for encapsulating an effective molecule in a lipid complex include a reverse phase evaporation method, a zwitterion (NaCl) hydration method, a cationic core hydration method, and a method with ethanol and calcium (see, Biomembr., 1468, 239-252 (2000)). A lipid complex in pharmaceutical composition of an embodiment encapsulating the double-stranded ribonucleic acid can be prepared by using any of these methods known in the art.

A lipid complex in an embodiment comprising the double-stranded ribonucleic acid can be prepared by, for example, mixing a lipid solution containing the cationic lipid and at least one lipid selected from the group consisting of neutral lipid, polyethylene glycol-modified lipid, and sterol, and an acidic buffer containing the double-stranded ribonucleic acid. By using such a method, a lipid complex the inside of which is filled with a core of the double-stranded ribonucleic acid and the lipids can be obtained. A lipid complex of an embodiment comprising the double-stranded ribonucleic acid may contain the cationic lipid and at least one lipid selected from the group consisting of neutral lipid, polyethylene glycol-modified lipid, and sterol.

A lipid complex of an embodiment comprising the double-stranded ribonucleic acid can be produced by using a method including: a step (a) of mixing a polar organic solvent-containing aqueous solution containing (I) the cationic lipid and (II) at least one lipid selected from the group consisting of neutral lipid, polyethylene glycol-modified lipid, and sterol, and an aqueous solution containing (III) the double-stranded ribonucleic acid to obtain a mixed solution; and a step (b) of reducing the content of the polar organic solvent in the mixed solution.

Through the electrostatic interaction between the double-stranded ribonucleic acid and the cationic lipid, each being water-soluble, and the hydrophobic interaction among the lipids, a lipid complex encapsulating the double-stranded ribonucleic acid in a fine particle comprising the lipids can be formed. For example, a lipid complex can be formed by reducing the content of the polar organic solvent in the mixed solution to change the solubility of the lipid component containing (I) the cationic lipid and (II) at least one lipid selected from the group consisting of neutral lipid, polyethylene glycol-modified lipid, and sterol in the polar organic solvent-containing aqueous solution. Examples of the polar organic solvent include alcohol such as ethanol.

First, in the step (a), a polar organic solvent-containing aqueous solution containing (I) the cationic lipid and (II) at least one lipid selected from the group consisting of neutral lipid, polyethylene glycol-modified lipid, and sterol dissolved therein is mixed with an aqueous solution containing (III) the double-stranded ribonucleic acid to obtain a mixed solution. The concentration of the polar organic solvent in the polar organic solvent-containing aqueous solution is not particularly limited as long as conditions for dissolving lipid molecules are satisfied even after mixing with the aqueous solution containing the double-stranded ribonucleic acid. The concentration of the polar organic solvent in the mixed solution obtained in the step (a) can be 0.1 to 60% by weight. The aqueous solution containing (III) the double-stranded ribonucleic acid is obtained by, for example, dissolving the double-stranded ribonucleic acid in an acidic buffer.

Subsequently, in the step (b), the content of the polar organic solvent is reduced by adding water or the like to the mixed solution. Thereby, a lipid complex can be formed. It is preferred for efficient formation of the lipid complex to rapidly lower the content of the polar organic solvent. In an example, the concentration of the polar organic solvent in the final polar organic solvent-containing aqueous solution in the step (b) can be 0 to 5% by weight.

The mixed solution obtained in the step (a) may be subjected to dialysis to remove the polar organic solvent and substitute the solvent with a pharmaceutically acceptable medium. The content of the polar organic solvent in the solution decreases during the dialysis, by which a lipid complex can be formed.

By using the method for producing a composition of an embodiment, a lipid complex encapsulating the double-stranded ribonucleic acid in the inside of a fine particle can be obtained with high encapsulation efficiency.

Examples of the acidic buffer to dissolve the double-stranded ribonucleic acid therein include sulfate buffer, phosphate buffer, phthalate buffer, tartrate buffer, citrate buffer, formate buffer, oxalate buffer, and acetate buffer.

Examples of the solvent to dissolve the lipids therein include polar organic solvent such as alcohol, and the solvent may be, for example, ethanol, isopropanol, chloroform, or tert-butanol.

The method of the present embodiment for producing a pharmaceutical composition can further comprise adjusting the pH of a solution of the lipid complex. The adjustment of the pH of the solution is typically performed after inclusion of a double-stranded ribonucleic acid in the lipid complex. The adjusting step may be, for example, adjusting the pH of a solution of the lipid complex to 5.0 or less or 7.5 or more, or adjusting the pH of a solution of the lipid complex to 2.0 or more and 5.0 or less, or 7.5 or more and 11.0 or less, or adjusting the pH of a solution of the lipid complex to 2.0 or more and 5.0 or less, or adjusting the pH of a solution of the lipid complex to 7.5 or more and 11.0 or less, or adjusting the pH of a solution of the lipid complex to 7.5 or more or 10.0, or adjusting the pH of a solution of the lipid complex to 7.5 or more and 9.5 or less, or adjusting the pH of a solution of the lipid complex to 7.5 or more and 9.0 or less, or adjusting the pH of a solution of the lipid complex to 7.5 or more and 8.5 or less. In a particular embodiment, the adjusting step is adjusting the pH of a solution of the lipid complex to 7.5 or more and 8.5 or less.

The adjustment of the pH can be performed by using a known method, and may be performed, for example, by using an acidic aqueous solution of hydrochloric acid or the like or a basic aqueous solution of sodium hydroxide or the like, or performed by using buffer solution (buffer) of phosphate, citrate, acetate, tartrate, borate, or the like.

As described above, expression of complement C5 can be inhibited through RNAi by using the double-stranded ribonucleic acid comprised in the pharmaceutical composition of the present embodiment. In an embodiment, the pharmaceutical composition can comprise a pharmaceutically acceptable carrier in addition to the lipid complex comprising a double-stranded ribonucleic acid.

Examples of the pharmaceutically accepted carrier include liquid or solid fillers, diluent, excipients, production aids, and solvent-encapsulating materials.

The pharmaceutical composition in an embodiment may be, for example, in the form of powder obtained by removing solvent through freeze-drying or the like, or in the form of liquid. A pharmaceutical composition of an embodiment is a powder composition containing a lipid complex of any of the above-described embodiments. The powder composition may be prepared by removing solvent from a composition in the form of liquid (dispersion), for example, through filtration or centrifugation, or prepared by freeze-drying the dispersion. In the case that the pharmaceutical composition is in the form of powder, the pharmaceutical composition can be suspended or dissolved in a pharmaceutically acceptable medium before use and used as an injection. A pharmaceutical composition of an embodiment is a liquid composition containing a lipid complex of any of the above-described embodiments and a pharmaceutically acceptable medium. In the case that the pharmaceutical composition is in the form of liquid, the pharmaceutical composition can be directly used as an injection, or suspended or dissolved in a pharmaceutically acceptable medium and used as an injection.

By administering to a subject in need thereof, the pharmaceutical composition of the present embodiment can inhibit expression of complement C5 in the subject through RNAi. Here, the “subject in need thereof” refers to a subject presenting with a disease or disorder associated with expression or activity of the C5 gene, or a subject determined to have a high risk of development thereof

In some embodiments, the double-stranded ribonucleic acid that the pharmaceutical composition of the present embodiment comprises can suppress expression of complement C5, and hence the pharmaceutical composition of the present embodiment can be useful for treating paroxysmal nocturnal hemoglobinuria (PNH) and atypical hemolytic uremic syndrome (aHUS). Thus, in other embodiments, the invention is encompassed a method for treating paroxysmal nocturnal hemoglobinuria or atypical hemolytic uremic syndrome, the method including administering a therapeutically effective amount of a pharmaceutical composition of an embodiment to a subject.

The subject to administer a pharmaceutical composition of an embodiment is not limited, and, for example, the invention can be used to humans or non-human mammals (such as monkeys, mice, rats, rabbits, cows, horses, goats).

The method for administering a pharmaceutical composition of an embodiment to a subject (such as the route of administration, dose, frequency of administration per day, timing of administration) is not limited, and can be appropriately determined by one of ordinary skill in the art (e.g., physicians) in accordance with the health condition of a subject, the degree of a disease, the type of an agent to be used in combination.

### (Administration Method)

The mode of administration of a pharmaceutical composition of an embodiment is not particularly limited, and may be parenteral administration, and examples thereof include intravenous administration, intramuscular administration, subcutaneous administration, intradermal administration, and intrathecal administration.

A pharmaceutical composition of an embodiment can be administered in an amount enough to inhibit complement C5 depending on the mode of administration. The dose of a pharmaceutical composition of an embodiment may be, for example, 0.01 mg to 100 mg, or 0.1 mg to 50 mg, or 0.3 mg to 10 mg, per kg body weight of a subject.

One of ordinary skill in the art understand that the present invention may be implemented with appropriate combination of any one or more of all the embodiments described herein, unless the combination causes any technical contradiction. In addition, one of ordinary skill in the art understand that it would be preferred to implement the present invention with appropriate combination of any of all the preferred or advantageous embodiments described herein, unless the combination causes any technical contradiction.

All of the contents disclosed in the literatures mentioned herein are incorporated by reference in their entirety, and one of ordinary skill in the art can cite and understand related contents disclosed in the literatures as a part of the present specification in accordance with the context of the present specification, without departing from the spirit and scope of the present invention.

The literatures cited herein are provided only for the purpose of disclosing related art before the filing date of the present application, and should not be interpreted as admission that the present inventors have no right of priority to the disclosures because of any prior invention or for any other reason. All of the descriptions in the literatures are based on information which was available for the applicant, and by no means constitute admission that the described contents are correct.

The terms used herein are for describing specific embodiments, and not intended to limit the invention.

The term “comprise” used herein is intended to indicate the presence of a mentioned matter (e.g., a member, a step, an element, or a number) unless the context apparently requires different understanding, and does not exclude the presence of another matter (e.g., a member, a step, an element, or a number). The term “consist of” encompasses embodiments described with the term(s) “consist of” and/or “consist essentially of”.

Unless otherwise defined, all terms used herein (including technical terms and scientific terms) have the same meaning as commonly understood by one of ordinary skill in the art to which the present invention pertains. Each of the terms used herein should be interpreted to have a meaning consistent with that in the present specification and in the fields of related art unless otherwise specified, and should not be interpreted with respect to an idealized or excessively literal meaning

While terms such as “first” and “second” are used to represent various elements, it is to be understood that such elements should not be limited by the terms themselves. The terms are used only to distinguish one element from another element, and, for example, it is acceptable without departing from the scope of the present invention to express a first element as “second element” and to express a second element as “first element”, similarly.

Numerical values used herein to indicate component contents, numerical ranges, and so forth should be understood to be modified with the term “approximately”, unless otherwise specified. For example, “4° C.” is understood to refer to “approximately 4° C”, unless otherwise specified, and, needless to say, one of ordinary skill in the art can rationally understand the allowance in accordance with the common general knowledge and the context of the present specification. In this case, “approximately” is understood with applying the common round-off technique considering significant figures of each numerical value. Specifically, “approximately” is used to additionally include numerical values rounded to the nearest integer, wherein, for example, the range corresponding to “approximately 4” is from 3.5 to 4.4, and used to additionally include numerical values rounded to one decimal place, wherein, for example, the range corresponding to “approximately 4.5” is from 4.45 to 4.54. Cases of three or more decimal places are similarly understood.

Unless the context clearly indicates otherwise, embodiments with a singular form as used herein and in the claims are to be understood to allow the plural form, and vice versa, as long as no technical contradiction is caused.

Hereinafter, the present invention will be described in more detail with reference to Examples. However, the present invention can be realized in various embodiments, and should not be interpreted to be limited to Examples described below. One of ordinary skill in the art can implement the present invention with various modifications, additions, deletions, substitutions, and so forth, without changing the spirit or scope of the present invention.

### EXAMPLES

### Example 1: In-Vitro Screening for Single Administration (1)

### (Preparation of Double-Stranded Nucleic Acids)

Sense strands and antisense strands listed in Table 2 were synthesized by using the phosphoramidite method, and then annealed to synthesize double-stranded nucleic acids (GeneDesign, Inc.). Abbreviations in the sequences are as shown in Table 1. Each double-stranded nucleic acid synthesized had a hydroxy group instead of a phosphate group at each 3′-terminal

TABLE 1

| Abbreviation | Nucleotide |
|---|---|
| A | Adenosine-3′-phosphate |
| U | Uridine-3′-phosphate |
| G | Guanosine-3′-phosphate |
| C | Cytidine-3′-phosphate |
| a | 2′-O-methyladenosine-3′-phosphate |
| u | 2′-O-methyluridine-3′-phosphate |
| g | 2′-O-methylguanosine-3′-phosphate |
| c | 2′-O-methylcytidine-3′-phosphate |
| dT | Deoxy-thymidine |
| (idT) | Inverted deoxy-thymidine (inverted dT) |
| \hat{ } | Phosphorothioate bond |

TABLE 2

| | Numbers of | | | | | |
|---|---|---|---|---|---|---|
| | nucleotides | | | | | |
| | in sense | Sense strand | | Antisense strand | | Target |
| Double | strand/anti- | | SEQ | | SEQ | site in |
| strand ID | sense strand | Sequence (5′→3′) | ID NO | Sequence (5′→3′) | ID NO | NM_001735.2 |
| SiRNA-001 | 21/21 | AGGcAAAGGuGuucAAAGAdT^{∧}dT | 1 | UCUUUGAAcACCUUUGCCUdT^{∧}dT | 2 | 2477-2495 |
| SiRNA-002 | 21/21 | cuGucuuAAcuuucAuAGAdT^{∧}dT | 3 | UCuAUGAAAGUuAAGAcAGdT^{∧}dT | 4 | 506-524 |
| siRNA-003 | 21/21 | uAGcAuGuGccAGcuAcAAdT^{∧}dT | 5 | UUGuAGCUGGcAcAUGCuAdT^{∧}dT | 6 | 4238-4256 |
| siRNA-004 | 21/21 | cuGuGAuuGGAAuuAGAAAdT^{∧}dT | 7 | UUUCuAAUUCCAAUGAcAGdT^{∧}dT | 8 | 3473-3491 |
| SiRNA-006 | 21/21 | AAGGCAAAGGuGuucAAAGdT^{∧}dT | 9 | CUUUGAACACCUUUGCCUUdT^{∧}dT | 10 | 2476-2494 |
| siRNA-007 | 21/21 | GAAAGGAAcuGuuuAcAAcdT^{∧}dT | 11 | GUUGUAAACAGUUCCUUUCdT^{∧}dT | 12 | 2553-2571 |
| siRNA-008 | 21/21 | uGGuAuAuGuGuuGcuGAudT^{∧}dT | 13 | AUGAGCAAcAcAuAuACcAdT^{∧}dT | 14 | 2451-2469 |
| siRNA-009 | 21/21 | AcuGucuuAAcuuucAuAGdT^{∧}dT | 15 | CuAUGAAAGUuAAGAcAGUdT^{∧}dT | 16 | 505-523 |
| SiRNA-010 | 21/21 | GuGccAGcuAcAAGcccAGdT^{∧}dT | 17 | CUGGGCUUGuAGCUGGcACHT^{∧}dT | 18 | 4244-4262 |
| siRNA-011 | 21/21 | AAGGAAcuGuuuAcAAcuAdT^{∧}dT | 19 | uAGUUGUAAACAGUUCCUUdT^{∧}dT | 20 | 2555-2573 |
| siRNA-012 | 21/21 | uccucuGGAAAuuGGccuudT^{∧}dT | 21 | AAGGCCAAUUUCcAGAGGAdT^{∧}dT | 22 | 2733-2751 |
| siRNA-013 | 21/21 | uuGAAAGGAAcuGuuuAcAdT^{∧}dT | 23 | UGuAAAcAGUUCCUUUCAAdT^{∧}dT | 24 | 2551-2569 |
| SiRNA-014 | 21/21 | AAAGGAAcuGuuuAcAAcudT^{∧}dT | 25 | AGUUGUAAACAGUUCCUUUdT^{∧}dT | 26 | 2554-2572 |
| SiRNA-015 | 21/21 | AGGAAcuGuuuAcAAcuAudT^{∧}dT | 27 | AuAGUUGUAAACAGUUCCUdT^{∧}dT | 28 | 2556-2574 |
| siRNA-016 | 21/21 | uAcAcuGAAGcAuuuGAuGdT^{∧}dT | 29 | CAUCAAAUGCUUCAGUGuAdT^{∧}dT | 30 | 166-184 |
| siRNA-017 | 21/21 | CAcuGAAGcAuuuGAuGcAdT^{∧}dT | 31 | UGCAUCAAAUGCUUcAGUGdT^{∧}dT | 32 | 168-186 |
| SiRNA-018 | 21/21 | cuGAAGcAuuuGAuGcAAcdT^{∧}dT | 33 | GUUGCAUCAAAUGCUUcAGdT^{∧}dT | 34 | 170-188 |
| siRNA-019 | 21/21 | uucuGcAAcuGAAuucGAudT^{∧}dT | 35 | AUCGAAUUCAGUUGGAGAAdT^{∧}dT | 36 | 4412-4430 |
| siRNA-020 | 21/21 | uGAAAGGAAcuGuuuAcAAdT^{∧}dT | 37 | UUGuAAACAGUUCCUUUcAdT^{∧}dT | 38 | 2552-2570 |
| siRNA-021 | 21/21 | AcuGAAGcAuuuGAuGcAAdT^{∧}dT | 39 | UUGCAUCAAAUGCUUCAGUdT^{∧}dT | 40 | 169-187 |
| siRNA-022 | 21/21 | cAuAcAGAcAAAccuGuuudT^{∧}dT | 41 | AAACAGGUUUGUCUGUAUGdT^{∧}dT | 42 | 415-433 |
| siRNA-023 | 21/21 | AAACAACAAGuAccuuuAudT^{∧}dT | 43 | AuAAAGGUACUUGUUGUUUdT^{∧}dT | 44 | 984-1002 |
| siRNA-024 | 21/21 | AuAcAGAcAAAccuGuuuAdT^{∧}dT | 45 | uAAAcAGGUUUGUCUGuAUdT^{∧}dT | 46 | 416-434 |
| siRNA-025 | 21/21 | GGuAuAuGuGuuGouGAuAdT^{∧}dT | 47 | uAUcAGCAAcAcAuAuACCdT^{∧}dT | 48 | 2452-2470 |
| siRNA-026 | 21/21 | ucAGAAAGucuGuGAAGGAdT^{∧}dT | 49 | UCCUUcAcAGACUUUCUGAdT^{∧}dT | 50 | 4578-4596 |
| SiRNA-027 | 21/21 | ucuccAGGccAAAcuGuGudT^{∧}dT | 51 | AcACAGUUUGGCCUGGAGAdT^{∧}dT | 52 | 1777-1795 |
| siRNA-028 | 21/21 | AcAAcAAGuAccuuuAuAudT^{∧}dT | 53 | AuAuAAAGGuACUUGUUGUdT^{∧}dT | 54 | 986-1004 |
| siRNA-029 | 21/21 | CAACAAGuAccuuuAuAuudT^{∧}dT | 55 | AAuAuAAAGGuACUUGUUGdT^{∧}dT | 56 | 987-1005 |
| siRNA-030 | 21/21 | AuucuccAGGccAAAcuGudT^{∧}dT | 57 | AcAGUUUGGCCUGGAGAAUdT^{∧}dT | 58 | 1775-1793 |
| siRNA-031 | 21/21 | GuGGcAAccAGcuccAGGudT^{∧}dT | 59 | ACCUGGAGCUGGUUGCCACdT^{∧}dT | 60 | 1730-1748 |
| siRNA-032 | 21/21 | AAGAGAcAucuGAcuuGGAdT^{∧}dT | 61 | UCCAAGUCAGAUGUCUCUUdT^{∧}dT | 62 | 1226-1244 |
| siRNA-033 | 21/21 | AuucuGcAAcuGAAuucGAdT^{∧}dT | 63 | UCGAAUUCAGUUGCAGAAUdT^{∧}dT | 64 | 4411-4429 |
| SiRNA-034 | 21/21 | uuccucuGGAAAuuGGccudT^{∧}dT | 65 | AGGCCAAUUUCCAGAGGAAdT^{∧}dT | 66 | 2732-2750 |
| siRNA-035 | 21/21 | AACAAcAAGuAccuuuAuAdT^{∧}dT | 67 | uAuAAAGGuACUUGUUGUUdT^{∧}dT | 68 | 985-1003 |
| siRNA-036 | 21/21 | AAuAuGuccucucucccuAdT^{∧}dT | 69 | uAGGGAGAGAGGAcAuAUUdT^{∧}dT | 70 | 1067-1085 |
| siRNA-037 | 21/21 | AcucAcuAuAAuuAcuuGAdT^{∧}dT | 71 | UcAAGUAAUuAuAGUGAGUdT^{∧}dT | 72 | 1519-1537 |
| siRNA-038 | 21/21 | AuAAcucAcuAuAAuuAcudT^{∧}dT | 73 | AGUAAUuAuAGUGAGUuAUdT^{∧}dT | 74 | 1516-1534 |
| SiRNA-039 | 21/21 | AAAuAuGuccucucuccoudT^{∧}dT | 75 | AGGGAGAGAGGACAuAUUUdT^{∧}dT | 76 | 1066-1084 |
| SiRNA-040 | 21/21 | AAGAuAuuuuuAuAAuAAAdT^{∧}dT | 77 | UUuAUuAuAAAAAuAUCUUdT^{∧}dT | 78 | 876-894 |
| siRNA-042 | 21/21 | AAAAuAAcucAcuAuAAuudT^{∧}dT | 79 | AAUuAuAGUGAGUuAUUUUdT^{∧}dT | 80 | 1513-1531 |
| siRNA-043 | 21/21 | AAAuAAcucAcuAuAAuuAdT^{∧}dT | 81 | uAAUuAuAGUGAGUuAUUUdT^{∧}dT | 82 | 1514-1532 |
| siRNA-044 | 21/21 | GuGuuAAAAuGucuGcuGudT^{∧}dT | 83 | AcAGcAGAcAUUUuAAcACdT^{∧}dT | 84 | 2597-2615 |
| siRNA-045 | 21/21 | AAAAuGuuuuuGucAAGuAdT^{∧}dT | 85 | uACUUGACAAAAAcAUUUUdT^{∧}dT | 86 | 4742-4760 |
| Mock | 21/21 | cuuAcGcuGAGuAcuucGAdT^{∧}dT | 87 | UCGAAGuACUcAGCGuAAGdT^{∧}dT | 88 | — |

### (In-Vitro Screening)

Each of the double-stranded nucleic acids listed in Table 2 in combination with the transfection reagent Lipofectamine RNAiMax (from Invitrogen, catalog number: 13778150) was diluted with an Opti-MEM medium (from Gibco, catalog number: 31985062) to prepare siRNA/RNAiMax mixed solution with a final concentration of 3 nM double-stranded nucleic acid and 0.3% RNAiMax. The siRNA/RNAiMax mixed solution was aliquoted into 20 μL portions in wells of a 96-well culture plate, and Hep3B cells (obtained from ATCC) as cell lines derived from human liver cancer were seeded in each well at 20000 cells/80 μL/well, and cultured under conditions of 37° C. and 5% CO₂ overnight. From the cultured cells, a template lysate for real-time PCR was prepared by using a CellAmp (registered trademark) Direct RNA Prep Kit for RT-PCR (Real Time) (from Takara Bio Inc., catalog number: 3732) and Proteinase K (from Takara Bio Inc., catalog number: 9034) in accordance with a protocol provided by Takara Bio Inc. Thereafter, cDNA was prepared by using a PrimeScript (registered trademark) RT Master Mix (Perfect Real Time) (from Takara Bio Inc., catalog number: RR036A) in accordance with a protocol provided by Takara Bio Inc. Further, Ct values were measured for the target gene human C5 and the endogenous control gene human GAPDH (glyceraldehyde-3-phosphate dehydrogenase) by using an EagleTaq Universal Master Mix (ROX) (from Roche Diagnostics K.K., catalog number: 07260296190) and a TaqMan probe (from Applied Biosystems, C5: Hs00156197_m1; GAPDH: Hs02758991_g1) with an ABI7900HT real-time PCR system (from Applied Biosystems) in accordance with a protocol provided by Applied Biosystems. The C5 mRNA expression level in the case that Hep3B cells were treated only with the transfection reagent without addition of siRNA was defined as 100%, and a C5 mRNA residual rate (relative value) was calculated for each introduction of siRNA by using a calibration curve method. As a negative control, Mock which does not cross over with any human gene was used.

The results are shown in Table 3.

TABLE 3

| | C5 mRNA residual rate |
|---|---|
| Double | (n = 3, average) |
| strand ID | (3 nM siRNA) |
| siRNA-001 | 18% |
| siRNA-002 | 43% |
| siRNA-003 | 107% |
| siRNA-004 | 35% |
| siRNA-006 | 37% |
| siRNA-007 | 28% |
| siRNA-008 | 18% |
| siRNA-009 | 89% |
| siRNA-010 | 107% |
| siRNA-011 | 107% |
| siRNA-012 | 37% |
| siRNA-013 | 53% |
| siRNA-014 | 64% |
| siRNA-015 | 125% |
| siRNA-016 | 35% |
| siRNA-017 | 26% |
| siRNA-018 | 48% |
| siRNA-019 | 27% |
| siRNA-020 | 42% |
| siRNA-021 | 74% |
| siRNA-022 | 39% |
| siRNA-023 | 101% |
| siRNA-024 | 41% |
| siRNA-025 | 125% |
| siRNA-026 | 41% |
| siRNA-027 | 143% |
| siRNA-028 | 61% |
| siRNA-029 | 40% |
| siRNA-030 | 151% |
| siRNA-031 | 96% |
| siRNA-032 | 73% |
| siRNA-033 | 70% |
| siRNA-034 | 121% |
| siRNA-035 | 54% |
| siRNA-036 | 119% |
| siRNA-037 | 119% |
| siRNA-038 | 47% |
| siRNA-039 | 100% |
| siRNA-040 | 111% |
| siRNA-042 | 112% |
| siRNA-043 | 53% |
| siRNA-044 | 87% |
| siRNA-045 | 115% |
| Mock | 116% |
| Lipofection only | 100% |

### Example 2: In-Vitro Screening for Single Administration (2)

### (Preparation of Double-Stranded Nucleic Acids)

Sense strands and antisense strands listed in Table 4 were synthesized by using the phosphoramidite method, and then annealed to synthesize double-stranded nucleic acids (GeneDesign, Inc.).

TABLE 4

| | Numbers of | | | | |
|---|---|---|---|---|---|
| | nucleotides | | | | |
| | in sense | | | | |
| | strand/ | Sense strand | | Antisense strand | |
| Double | antisense | | SEQ | | SEQ |
| strand ID | strand | Sequence (5′→3′) | ID NO | Sequence (5′→3′) | ID NO |
| siRNA-001 | 21/21 | AGGcAAAGGuGuucAAAGAdT^{∧}dT | 89 | UCUUUGAAcACCUUUGCCUdT^{∧}dT | 90 |
| siRNA-001-02 | 19/19 | AGGcAAAGGuGuucAAAGA | 91 | UCUUUGAAcACCUUUGCCU | 92 |
| SiRNA-001-08 | 21/21 | A^{∧}GGcAAAGGuGuucAAAGAuu | 93 | U^{∧}CUUUGAAcACCUUUGCCUuu | 94 |
| siRNA-001-09 | 21/21 | A^{∧}G^{∧}GcAAAGGuGuucAAAGAuu | 95 | U^{∧}C^{∧}UUUGAAcACCUUUGCCUuu | 96 |
| SiRNA-001-10 | 21/21 | A^{∧}GGcAAAGGuGuucAAAGA^{∧}u^{∧}u | 97 | U^{∧}CUUUGAAcACCUUUGCCU^{∧}u?^{∧}u | 98 |
| SiRNA-001-11 | 19/21 | AGGcAAAGGuGuucAAAGA | 99 | UCUUUGAAcACCUUUGCCUuu | 100 |
| siRNA-001-12 | 21/21 | AuAGGcAAAGGuGuucAAAGA | 101 | UCUUUGAAcACCUUUGCCUuu | 102 |
| SiRNA-001-13 | 22/21 | uAuAGGcAAAGGuGuucAAAGA | 103 | UCUUUGAAcACCUUUGCCUuu | 104 |
| siRNA-006 | 21/21 | AAGGcAAAGGuGuucAAAGdT^{∧}dT | 105 | CUUUGAAcACCUUUGCCUUdT^{∧}dT | 106 |
| SiRNA-006-02 | 19/19 | AAGGcAAAGGuGuucAAAG | 107 | CUUUGAAcACCUUUGCCUU | 108 |
| siRNA-007 | 21/21 | GAAAGGAAcuGuuuAcAAcdT^{∧}dT | 109 | GUUGuAAAcAGUUCCUUUCdT^{∧}dT | 110 |
| SiRNA-007-02 | 19/19 | GAAAGGAAcuGuuuAcAAc | 111 | GUUGuAAAcAGUUCCUUUC | 112 |
| siRNA-008 | 21/21 | uGGuAuAuGuGuuGcuGAudT^{∧}dT | 113 | AUcAGcAAcAcAuAuACcAdT^{∧}dT | 114 |
| SiRNA-008-02 | 19/19 | uGGuAuAuGuGuuGcuGAu | 115 | AUcAGcAAcAcAuAuACcA | 116 |
| SiRNA-008-08 | 21/21 | u^{∧}GGuAuAuGuGuuGcuGAuuu | 117 | A^{∧}UcAGcAAcAcAuAuACcAuu | 118 |
| siRNA-008-09 | 21/21 | u^{∧}G^{∧}GuAuAuGuGuuGcuGAuuu | 119 | A^{∧}U^{∧}cAGcAAcAcAuAuACcAuu | 120 |
| SiRNA-008-10 | 21/21 | u^{∧}GGuAuAuGuGuuGcuGAu^{∧}u^{∧}u | 121 | A^{∧}UcAGcAAcAcAuAuACcA^{∧}u^{∧}u | 122 |
| SiRNA-008-11 | 19/21 | uGGuAuAuGuGuuGcuGAu | 123 | AUcAGcAAcAcAuAuACcAuu | 124 |
| siRNA-008-12 | 21/21 | AuuGGuAuAuGuGuuGcuGAu | 125 | AUcAGcAAcAcAuAuACcAuu | 126 |
| SiRNA-008-13 | 22/21 | uAuuGGuAuAuGuGuuGcuGAu | 127 | AUcAGcAAcAcAuAuACcAuu | 128 |
| SiRNA-008-14 | 23/21 | uAuuGGuAuAuGuGuuGcuGAu(idT) | 129 | AUcAGcAAcAcAuAuACcAuu | 130 |
| siRNA-008-22 | 21/21 | uGGuAuAuGuGuuGCuGAuuu | 131 | AUcAGcAAcAcAuAuACcAuu | 132 |
| siRNA-008-23 | 21/21 | uGGuAuAuGuGuuGcUGAuuu | 133 | AUcAGcAAcAcAuAuACcAuu | 134 |
| SiRNA-008-29 | 20/20 | AuuGGuAuAuGuGuuGcuGA | 135 | UcAGcAAcAcAuAuACcAuu | 136 |
| siRNA-008-30 | 20/20 | AuGGuAuAuGuGuuGcuGAu | 137 | AUcAGcAAcAcAuAuACcuu | 138 |
| SiRNA-008-31 | 19/23 | uGGuAuAuGuGuuGcuGAu | 139 | AUcAGcAAcAcAuAuACcAuuuu | 140 |
| SiRNA-008-32 | 19/23 | uGGuAuAuGuGuuGCuGAu | 141 | AUcAGcAAcAcAuAuACcAuuuu | 142 |
| SiRNA-008-33 | 19/23 | uGGuAuAuGuGuuGCuGAu | 143 | AUcAGcAAcAcAuAuACcAuuaa | 144 |
| siRNA-008-34 | 19/23 | uGGuAuAuGuGuuGCuGAu | 145 | AUcAGcAAcAcAuAuACcAuu^{∧}a^{∧}a | 146 |
| SiRNA-008-35 | 19/23 | uGGuAuAuGuGuuGCuGAu | 147 | AUcAGcAAcAcAuAuACcA^{∧}u^{∧}uaa | 148 |
| siRNA-008-36 | 19/23 | uGGuAuAuGuGuuGCuGAu | 149 | a^{∧}UcAGcAAcAcAuAuACcA^{∧}u^{∧}uaa | 150 |
| SiRNA-008-37 | 19/23 | uGGuAuAuGuGuuGCuGAu | 151 | aUcAGcAAcAcAuAuACcAuuuu | 152 |
| siRNA-008-38 | 19/23 | u?GGuAuAuGuGuuGCuGAu | 153 | AUcAGcAAcAcAuAuACcA^{∧}u^{∧}uaa | 154 |
| siRNA-038 | 21/21 | AuAAcucAcuAuAAuuAcudT^{∧}dT | 155 | AGuAAUuAuAGUGAGUuAUdT^{∧}dT | 156 |
| siRNA-038-02 | 19/19 | AuAAcucAcuAuAAuuAcu | 157 | AGuAAUuAuAGUGAGUuAU | 158 |
| Mock | 21/21 | cuuAcGouGAGuAcuucGAdT^{∧}dT | 87 | UCGAAGuACUcAGCGuAAGdT^{∧}dT | 88 |

### (In-Vitro Screening)

A test was performed to measure Ct values for the target gene human C5 and the endogenous control gene human GAPDH in cultured Hep3B cells in the same manner as in Example 1, except that siRNA/RNAiMax mixed solution was prepared with a final concentration of 1 nM double-stranded nucleic acid and 0.3% RNAiMax. As in Example 1, the C5 mRNA expression level in the case of Lipofection only was defined as 100%, and a C5 mRNA residual rate (relative value) was calculated for each introduction of siRNA.

The results are shown in Table 5. Lowered C5 mRNA residual rates were found for all of the double-stranded nucleic acids except siRNA-008-29, demonstrating that expression of C5 was suppressed.

TABLE 5

| | C5 mRNA residual rate |
|---|---|
| | (n = 3, average) |
| Double strand ID | (1 nM siRNA) |
| siRNA-001 | 36% |
| siRNA-001-02 | 44% |
| siRNA-001-08 | 42% |
| siRNA-001-09 | 42% |
| siRNA-001-10 | 44% |
| siRNA-001-11 | 45% |
| siRNA-001-12 | 39% |
| siRNA-001-13 | 45% |
| siRNA-006 | 43% |
| siRNA-006-02 | 40% |
| siRNA-007 | 44% |
| siRNA-007-02 | 50% |
| siRNA-008 | 34% |
| siRNA-008-02 | 37% |
| siRNA-008-08 | 37% |
| siRNA-008-09 | 41% |
| siRNA-008-10 | 45% |
| siRNA-008-11 | 38% |
| siRNA-008-12 | 39% |
| siRNA-008-13 | 46% |
| siRNA-008-14 | 42% |
| siRNA-008-22 | 37% |
| siRNA-008-23 | 36% |
| siRNA-008-29 | 119% |
| siRNA-008-30 | 33% |
| siRNA-008-31 | 36% |
| siRNA-008-32 | 39% |
| siRNA-008-33 | 36% |
| siRNA-008-34 | 32% |
| siRNA-008-35 | 33% |
| siRNA-008-36 | 37% |
| siRNA-008-37 | 34% |
| siRNA-008-38 | 38% |
| siRNA-038 | 43% |
| siRNA-038-02 | 54% |
| Mock | 90% |
| Lipofection only | 100% |

### Example 3: In-Vivo Screening (Sequence Finding)

(Preparation of siRNA-LNPs)

Each siRNA listed in Table 6 was dissolved in 10 mM sodium citrate (pH 4.0) to prepare diluted siRNA solution. Lipid solution was prepared by dissolving 2-{9-oxo-9-[(3-pentyloctyl)oxy]nonyl}dodecyl 1-methylpiperidine-4-carboxylate, DSPC (NIPPON FINE CHEMICAL CO., LTD.), Cholesterol (NIPPON FINE CHEMICAL CO., LTD.), and MPEG2000-DMG (NOF CORPORATION) at a mole ratio of 60/10.5/28/1.5 in ethanol. Lipid Nanoparticles (LNPs) were obtained by mixing the diluted siRNA solution and the lipid solution at flow rates of 3 mL/min and 1 mL/min, respectively, with an siRNA/lipid weight ratio of 0.1. The external solution of the resulting LNP aqueous solution was substituted with PBS (pH 7.4) through dialysis by using a Float-A-Lyzer G2 (SPECTRUM, 100K MWCO). After the dialysis, the resultant was subjected to concentration and filtration sterilization for use in experiments. The siRNA concentration and encapsulation efficiency were measured by using a Quant-iT RiboGreen RNA Reagent (Invitrogen, Cat#R11491). For calculation of the encapsulation efficiency, the siRNA concentration measured after dilution with RNase Free Water was assumed as the concentration of siRNA present in the LNP external solution, and the siRNA concentration measured after dilution with 1% Triton X-100 was assumed as the total siRNA concentration of the formulation. The average particle size (Z-average) was measured by using a particle size analyzer (Malvern Panalytical Ltd., Zetasizer Nano ZS). Results of evaluation of product quality for the prepared LNPs are shown in Table 7.

TABLE 6

| | Numbers of | | | | |
|---|---|---|---|---|---|
| | nucleotides | | | | |
| | in sense | | | | |
| | strand/ | Sense strand | | Antisense strand | |
| Double | antisense | | SEQ | | SEQ |
| strand ID | strand | Sequence (5′→3′) | ID NO | Sequence (5′→3′) | ID NO |
| Mock | 21/21 | cuuAcGcuGAGuAcuucGAdT^{∧}dT | 87 | UCGAAGuACUcAGCGuAAGdT^{∧}dT | 88 |
| SiRNA-001-01 | 21/21 | AGGcAAAGGuGuucAAAGAuu | 161 | UCUUUGAAcACCUUUGCCUuu | 162 |
| SiRNA-007-01 | 21/21 | GAAAGGAAcuGuuuAcAAcuu | 163 | GUUGUAAAcAGUUCCUUUCuu | 164 |
| SiRNA-008-01 | 21/21 | uGGuAuAuGuGuuGcuGAuuu | 159 | AUcAGcAAcAcAuAuACcAuu | 160 |

TABLE 7

| | Encapsulation | Average particle | Polydispersity |
|---|---|---|---|
| Double strand ID | efficiency | size (nm) | index |
| Mock | >90% | 92 | 0.06 |
| siRNA-001-01 | >90% | 90 | 0.08 |
| siRNA-007-01 | >90% | 87 | 0.06 |
| siRNA-008-01 | >90% | 88 | 0.1 |

### (In-Vivo Screening)

LNPs encapsulating PBS or siRNA listed in Table 6 therein were intravenously administered to a BALB/c mouse (male, 6 weeks old, n=3 per group) from the tail vein at a dose of 0.1 mg/kg siRNA, and the blood and liver were sampled under anesthesia 5 days and 14 days after the administration. From the liver frozen with liquid nitrogen, Total RNA was purified by using an RNeasy Plus Mini Kit (Qiagen, Cat#74106) in accordance with a protocol provided by the manufacturer. Thereafter, cDNA was prepared by using a PrimeScript RT Master Mix (Perfect Real Time) (Takara Bio Inc., Cat#RR036A) in accordance with a protocol provided by the manufacturer. Further, Ct values were measured for the target gene mouse C5 and the endogenous control gene mouse GAPDH by using a TaqMan (registered trademark) Gene Expression Master Mix (Applied Biosystem, Cat#4369510) and a TaqMan probe (Applied Biosystems, C5: Mm01336776_g1; GAPDH: Mm99999915_g1)with an ABI7500 Fast (Applied Biosystems) in accordance with a protocol provided by the manufacturer. The liver C5 mRNA residual rate 5 days after the administration for the PBS administration group was defined as 100%, and a liver C5 mRNA residual rate (relative value) was calculated for each siRNA administration group by using the comparative Ct method. The results are shown in Table 8.

TABLE 8

| | Liver C5 mRNA residual | Liver C5 mRNA residual |
|---|---|---|
| | rate 5 days after | rate 14 days after |
| Double | administration | administration |
| strand ID | (n = 3, Average) | (n = 3, Average) |
| PBS | 100% | Not tested |
| Mock | 75% | 105% |
| siRNA-001-01 | 51% | 104% |
| siRNA-007-01 | 69% | 92% |
| siRNA-008-01 | 21% | 62% |

The blood sampled on each sampling day was centrifuged at 3000 rpm for 15 minutes, and then the heparin plasma as the supernatant was collected and stored at −80° C. Thereafter, the plasma Mouse C5 was quantified by ELISA. Specifically, the mouse anti-05 antibody BB5.1 (Hycult Biotech, Cat#HM1073-FS) as an immobilized antibody was diluted with PBS(−) (Wako Pure Chemical Industries, Ltd., #045-29795) to a final concentration of 2 μg/mL and added to an assay plate (Nunc, Cat#442404), and incubated at 4° C. overnight. Thereafter, blocking solution (PBS(−) (Wako Pure Chemical Industries, Ltd.) containing 1% BSA (R&D systems, Inc., Cat#DY995)) was added, and the resultant was incubated at room temperature for 1 hour. The blocking solution was discarded, and washing was performed three times with washing solution (PBS(−) (Wako Pure Chemical Industries, Ltd.) containing 0.02% Tween20). The washing solution was discarded, and the heparin plasma sample diluted with blocking solution was then added, and the resultant was incubated at room temperature for 5 hours. The plasma of the PBS administration group was used as a standard sample. The sample was discarded, and washing was then performed five times with washing solution, and a goat anti-human C5 antibody (Quidel Corporation, Cat#A306) diluted 4000-fold with blocking solution was added, and the resultant was incubated at room temperature for 1 hour. The antibody was discarded, and washing was then performed five times with washing solution, and an HRP-labeled donkey anti-goat IgG (H+L) (Jackson ImmunoResearch Inc., Cat#805-035-180) diluted 40000-fold with blocking solution was added, and the resultant was incubated at room temperature for 1 hour. The antibody was discarded, and washing was then performed five times with washing solution. Thereafter, equal amounts of TMB (3,3′,5,5′-tetramethylbenzidine) Peroxidase Substrate (Kirkegaard & Perry Laboratories, Inc., Cat#50-76-01) and Peroxidase Substrate Solution B (Kirkegaard & Perry Laboratories, Inc., Cat#50-65-00) were mixed together as detection reagent, which was added and allowed to develop color. H2504 (Wako Pure Chemical Industries, Ltd., Cat#198-09595) was added as quenching solution, and absorbance was then measured at 450 nm and 650 nm. Relative values for the samples as the plasma C5 concentration 5 days after the administration for the PBS administration group was defined as 100% are shown in Table 9.

TABLE 9

| | Blood C5 protein residual | Blood C5 protein residual |
|---|---|---|
| | rate 5 days after | rate 14 days after |
| Double | administration | administration |
| strand ID | (n = 3, Average) | (n = 3, Average) |
| PBS | 100% | Not tested |
| Mock | 94% | 96% |
| siRNA-001-01 | 44% | 87% |
| siRNA-007-01 | 79% | 103% |
| siRNA-008-01 | 22% | 48% |

### Example 4: In-Vitro Screening

Sense strands and antisense strands listed in Table 10 were synthesized by using the phosphoramidite method, and then annealed to synthesize double-stranded nucleic acids (GeneDesign, Inc.). A test was performed to measure Ct values for the target gene human C5 and the endogenous control gene human GAPDH in cultured Hep3B cells in the same manner as in Example 1, except that siRNA/RNAiMax mixed solution was prepared with a final concentration of 0.003 to 10 nM double-stranded nucleic acid and 0.3% RNAiMax. As in Example 1, the C5 mRNA expression level in the case of Lipofection only was defined as 100%, and a C5 mRNA residual rate (relative value) was calculated for each introduction of siRNA. The results are shown in Table 11.

TABLE 10

| | Numbers of | | | | |
|---|---|---|---|---|---|
| | nucleotides | | | | |
| | in sense | | | | |
| | strand/ | Sense strand | | Antisense strand | |
| Double | antisense | | SEQ | | SEQ |
| strand ID | strand | Sequence (5′→3′) | ID NO | Sequence (5′→3′) | ID NO |
| siRNA-008-01 | 21/21 | uGGuAuAuGuGuuGcuGAuuu | 159 | AUcAGcAAcAcAuAuACcAuu | 160 |
| siRNA-008-31 | 19/23 | uGGuAuAuGuGuuGcuGAu | 139 | AUcAGcAAcAcAuAuACcAuuuu | 140 |
| siRNA-008-33 | 19/23 | uGGuAuAuGuGuuGCuGAu | 143 | AUcAGcAAcAcAuAuACcAuuaa | 144 |
| siRNA-008-34 | 19/23 | uGGuAuAuGuGuuGCuGAu | 145 | AUcAGcAAcAcAuAuACcAuu^{∧}a^{∧}a | 146 |
| siRNA-008-35 | 19/23 | uGGuAuAuGuGuuGCuGAu | 147 | AUcAGcAAcAcAuAuACcA^{∧}u^{∧}uaa | 148 |
| Mock | 21/21 | cuuAcGcuGAGuAcuucGAdT^{∧}dT | 87 | UCGAAGuACUcAGCGuAAGdT^{∧}dT | 88 |

TABLE 11

| | Double | C5 mRNA residual rate |
|---|---|---|
| siRNA (nM) | strand ID | (n = 3, average) |
| 10 | siRNA-008-01 | 17% |
| 3 | siRNA-008-01 | 20% |
| 1 | siRNA-008-01 | 22% |
| 0.3 | siRNA-008-01 | 26% |
| 0.1 | siRNA-008-01 | 34% |
| 0.03 | siRNA-008-01 | 60% |
| 0.01 | siRNA-008-01 | 66% |
| 0.003 | siRNA-008-01 | 85% |
| 10 | siRNA-008-31 | 16% |
| 3 | siRNA-008-31 | 22% |
| 1 | siRNA-008-31 | 26% |
| 0.3 | siRNA-008-31 | 35% |
| 0.1 | siRNA-008-31 | 49% |
| 0.03 | siRNA-008-31 | 79% |
| 0.01 | siRNA-008-31 | 96% |
| 0.003 | siRNA-008-31 | 83% |
| 10 | siRNA-008-33 | 15% |
| 3 | siRNA-008-33 | 27% |
| 1 | siRNA-008-33 | 37% |
| 0.3 | siRNA-008-33 | 53% |
| 0.1 | siRNA-008-33 | 70% |
| 0.03 | siRNA-008-33 | 96% |
| 0.01 | siRNA-008-33 | 103% |
| 0.003 | siRNA-008-33 | 112% |
| 10 | siRNA-008-34 | 16% |
| 3 | siRNA-008-34 | 28% |
| 1 | siRNA-008-34 | 34% |
| 0.3 | siRNA-008-34 | 38% |
| 0.1 | siRNA-008-34 | 44% |
| 0.03 | siRNA-008-34 | 68% |
| 0.01 | siRNA-008-34 | 76% |
| 0.003 | siRNA-008-34 | 91% |
| 10 | siRNA-008-35 | 15% |
| 3 | siRNA-008-35 | 23% |
| 1 | siRNA-008-35 | 30% |
| 0.3 | siRNA-008-35 | 42% |
| 0.1 | siRNA-008-35 | 59% |
| 0.03 | siRNA-008-35 | 87% |
| 0.01 | siRNA-008-35 | 92% |
| 0.003 | siRNA-008-35 | 98% |
| 10 | Mock | 116% |
| — | Lipofection | 100% |
| | only | |

### Example 5: In-Vivo Screening (Overhang Modification)

(Preparation of siRNA-LNPs)

Lipid nanoparticles (LNPs) encapsulating siRNA therein were prepared in the same manner as in Example 3, except that siRNAs listed in Table 12 were used. Results of evaluation of product quality for the prepared LNPs are shown in Table 13.

| | Numbers of | | | | |
|---|---|---|---|---|---|
| | nucleotides | | | | |
| | in sense | | | | |
| | strand/ | Sense strand | | Antisense strand | |
| Double | antisense | | SEQ | | SEQ |
| strand ID | strand | Sequence (5′→3′) | ID NO | Sequence (5′→3′) | ID NO |
| Mock | 21/21 | cuuAcGcuGAGuAcuucGAdT^{∧}dT | 87 | UCGAAGuACUcAGCGuAAGdT^{∧}dT | 88 |
| siRNA-008 | 21/21 | uGGuAuAuGuGuuGcuGAudT^{∧}dT | 13 | AUcAGcAAcAcAuAuACcAdT^{∧}dT | 14 |
| siRNA-008-32 | 19/23 | uGGuAuAuGuGuuGCuGAu | 141 | AUcAGcAAcAcAuAuACcAuuuu | 142 |
| SiRNA-008-33 | 19/23 | uGGuAuAuGuGuuGCuGAu | 143 | AUcAGcAAcAcAuAuACcAuuaa | 144 |
| siRNA-008-34 | 19/23 | uGGuAuAuGuGuuGCuGAu | 145 | AUcAGcAAcAcAuAuACcAuu^{∧}a^{∧}a | 146 |
| siRNA-008-35 | 19/23 | uGGuAuAuGuGuuGCuGAu | 147 | AUcAGcAAcAcAuAuACcA^{∧}u^{∧}uaa | 148 |
| SiRNA-008-38 | 19/23 | u^{∧}GGuAuAuGuGuuGCuGAu | 153 | AUcAGcAAcAcAuAuACcA^{∧}u^{∧}uaa | 154 |

TABLE 13

| Double | Encapsulation | Average particle | Polydispersity |
|---|---|---|---|
| strand ID | efficiency | size (nm) | index |
| Mock | >90% | 76 | 0.11 |
| siRNA-008 | >90% | 68 | 0.01 |
| siRNA-008-32 | >90% | 68 | 0.03 |
| siRNA-008-33 | >90% | 69 | 0.05 |
| siRNA-008-34 | >90% | 70 | 0.06 |
| siRNA-008-35 | >90% | 71 | 0.02 |
| siRNA-008-38 | >90% | 70 | 0.01 |

### (In-Vivo Screening)

LNPs encapsulating PBS or siRNA listed in Table 12 therein were intravenously administered to a BALB/c mouse (male, 6 weeks old, n=3 per group) from the tail vein at a dose of 0.3 mg/kg siRNA, and the blood and liver were sampled under anesthesia 5 days, 14 days, and 21 days after the administration. From the liver frozen with liquid nitrogen, Total RNA was purified by using an RNeasy Plus Mini Kit (Qiagen, Cat#74106) in accordance with a protocol provided by the manufacturer. Thereafter, cDNA was prepared by using a PrimeScript RT Master Mix (Perfect Real Time) (Takara Bio Inc., Cat#RR036A) in accordance with a protocol provided by the manufacturer. Further, Ct values were measured for the target gene mouse C5 and the endogenous control gene mouse GAPDH by using a TaqMan (registered trademark) Gene Expression Master Mix (Applied Biosystem, Cat#4369510) and a TaqMan probe (Applied Biosystems, C5: Mm01336776_g1; GAPDH: Mm99999915_g1) with an ABI7500 Fast (Applied Biosystems) in accordance with a protocol provided by the manufacturer. The liver C5 mRNA residual rate on each day of measurement for the PBS administration group was defined as 100%, and liver C5 mRNA residual rates (relative values) were calculated for each siRNA administration group by using the comparative Ct method. The results are shown in Table 14.

TABLE 14

| | Liver C5 mRNA residual rate | | |
|---|---|---|---|
| | (n = 3, average) | | |
| Double | 5 days after | 14 days after | 21 days after |
| strand ID | administration | administration | administration |
| PBS | 100% | 100% | 100% |
| Mock | 90% | 119% | 82% |
| siRNA-008 | 22% | 35% | 60% |
| siRNA-008-32 | 22% | 23% | 46% |
| siRNA-008-33 | 17% | 37% | 47% |
| siRNA-008-34 | 16% | 26% | 35% |
| siRNA-008-35 | 12% | 20% | 47% |
| siRNA-008-38 | 14% | 24% | 40% |

The blood sampled on each sampling day was centrifuged at 3000 rpm for 15 minutes, and then the heparin plasma as the supernatant was collected and stored at ˜80° C. Thereafter, the plasma Mouse C5 was quantified by ELISA. Specifically, the mouse anti-05 antibody BB5.1 (Hycult Biotech, Cat#HM1073-FS) as an immobilized antibody was diluted with PBS(−) (Wako Pure Chemical Industries, Ltd., #045-29795) to a final concentration of 2 μg/mL and added to an assay plate (Nunc, Cat#442404), and incubated at 4° C. overnight. Thereafter, blocking solution (PBS(−) (Wako Pure Chemical Industries, Ltd.) containing 1% BSA (R&D systems, Inc., Cat#DY995)) was added, and the resultant was incubated at room temperature for 1 hour. The blocking solution was discarded, and washing was performed three times with washing solution (PBS(−) (Wako Pure Chemical Industries, Ltd.) containing 0.02% Tween20). The washing solution was discarded, and the heparin plasma sample diluted with blocking solution was then added, and the resultant was incubated at room temperature for 5 hours. The plasma of the PBS administration group was used as a standard sample. The sample was discarded, and washing was then performed five times with washing solution, and a goat anti-human C5 antibody (Quidel Corporation, Cat#A306) diluted 4000-fold with blocking solution was added, and the resultant was incubated at room temperature for 1 hour. The antibody was discarded, and washing was then performed five times with washing solution, and an HRP-labeled donkey anti-goat IgG (H+L) (Jackson ImmunoResearch Inc., Cat#805-035-180) diluted 40000-fold with blocking solution was added, and the resultant was incubated at room temperature for 1 hour. The antibody was discarded, and washing was then performed five times with washing solution. Thereafter, equal amounts of TMB (3,3′,5,5′-tetramethylbenzidine) Peroxidase Substrate (Kirkegaard & Perry Laboratories, Inc., Cat#50-76-01) and Peroxidase Substrate Solution B (Kirkegaard & Perry Laboratories, Inc., Cat#50-65-00) were mixed together as detection reagent, which was added and allowed to develop color. H₂SO₄ (Wako Pure Chemical Industries, Ltd., Cat#198-09595) was added as quenching solution, and absorbance was then measured at 450 nm and 650 nm. Relative values for the samples as the plasma C5 concentration on the day before the administration for the PBS administration group was defined as 100%, are shown in Table 15.

TABLE 15

| | Plasma C5 residual rate | | |
|---|---|---|---|
| | (n = 3, average) | | |
| Double | 5 days after | 14 days after | 21 days after |
| strand ID | administration | administration | administration |
| PBS | 98% | 97% | 101% |
| Mock | 106% | 104% | 108% |
| siRNA-008 | 8% | 21% | 47% |
| siRNA-008-32 | 9% | 15% | 42% |
| siRNA-008-33 | 7% | 29% | 50% |
| siRNA-008-34 | 4% | 11% | 29% |
| siRNA-008-35 | 7% | 19% | 49% |
| siRNA-008-38 | 5% | 17% | 42% |

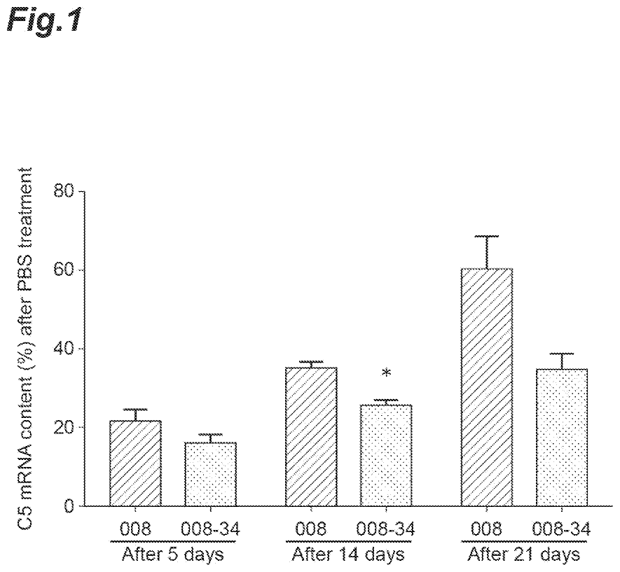

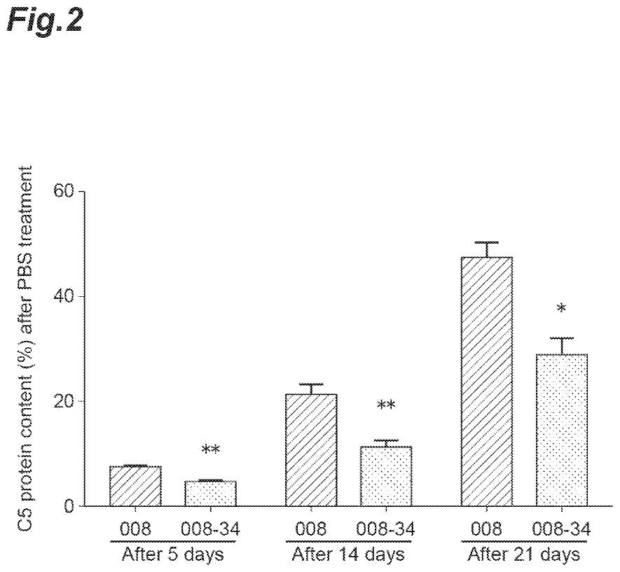

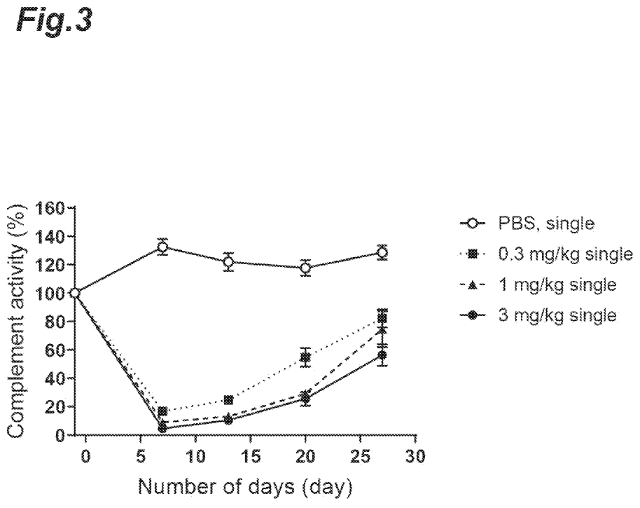

Liver C5 mRNA residual rates and plasma C5 concentrations 5 days, 14 days, and 21 days after the administration were quantified, and subjected to statistical analysis (unpaired T-test) for the siRNA-008-34 administration group to the siRNA-008 administration group. The results are shown in FIG. 1 and FIG. 2. Groups with a P value of 0.05 or lower were provided with * (asterisk), and groups with a P value of 0.01 or lower were provided with **.

### Example 6: In-Vitro Analysis (Sequence Walk)

Sense strands and antisense strands listed in Table 16 were synthesized by using the phosphoramidite method, and then annealed to synthesize double-stranded nucleic acids (GeneDesign, Inc.). As in Example 1, the C5 mRNA expression level in the case of Lipofection only was defined as 100%, and a C5 mRNA residual rate (relative value) was calculated for each introduction of siRNA. The results are shown in Table 17.

TABLE 16

| | Numbers of | | | | | |
|---|---|---|---|---|---|---|
| | nucleotides | | | | | |
| | in sense | | | | | |
| | strand/ | Sense strand | | Antisense strand | | Target site |
| Double | antisense | | SEQ | | SEQ | in |
| strand ID | strand | Sequence (5′→3′) | ID NO | Sequence (5′→3′) | ID NO | NM_001735.2 |
| Seq2449 | 21/21 | AcuGGuAuAuGuGuuGcuGdT^{∧}dT | 165 | cAGcAAcAcAuAuACcAGUdT^{∧}dT | 166 | 2449-2467 |
| Seq2450 | 21/21 | cuGGuAuAuGuGuuGcuGAdT^{∧}dT | 167 | UcAGcAAcAcAuAuACcAGdT^{∧}dT | 168 | 2450-2468 |
| Seq2451 | 21/21 | uGGuAuAuGuGuuGcuGAudT^{∧}dT | 13 | AUcAGcAAcAcAuAuACcAdT^{∧}dT | 14 | 2451-2469 |
| (= siRNA-008) | | | | | | |
| Seq2452 | 21/21 | GGuAuAuGuGuuGcuGAuAdT^{∧}dT | 169 | uAUcAGcAAcAcAuAuACCdT^{∧}dT | 170 | 2452-2470 |
| Seq2453 | 21/21 | GuAuAuGuGuuGcuGAuAcdT^{∧}dT | 171 | GuAUcAGcAAcAcAuAuACdT^{∧}dT | 172 | 2453-2471 |
| Mock | 21/21 | cuuAcGcuGAGuAcuucGAdT^{∧}dT | 87 | UCGAAGuACUcAGCGuAAGdT^{∧}dT | 88 | |

TABLE 17

| | | C5 mRNA residual rate |
|---|---|---|
| siRNA (nM) | Double strand ID | (n = 3, average) |
| 10 nM | Seq2449 | 107% |
| | Seq2450 | 105% |
| | Seq2451 | 12% |
| | (=siRNA-008) | |
| | Seq2452 | 111% |
| | Seq2453 | 20% |
| 1 nM | Seq2449 | 110% |
| | Seq2450 | 97% |
| | Seq2451 | 14% |
| | (=siRNA-008) | |
| | Seq2452 | 107% |
| | Seq2453 | 24% |
| 10 nM | Mock | 96% |
| — | Lipofection only | 100% |

### Example 7: Pharmacological Test (Hemolysis-Suppressing Effect)

(Preparation of siRNA-LNPs)

Lipid nanoparticles (LNPs) encapsulating siRNA therein were prepared in the same manner as in Example 3, except that siRNAs listed in Table 18 were used. Results of evaluation of product quality for the prepared LNPs are shown in Table 19.

TABLE 18

| | Numbers of | | | | |
|---|---|---|---|---|---|
| | nucleotides | | | | |
| | in sense | | | | |
| | strand/ | Sense strand | | Antisense strand | |
| Double | antisense | | SEQ | | SEQ |
| strand ID | strand | Sequence (5′→3′) | ID NO | Sequence (5′→3′) | ID NO |
| Mock | 21/21 | cuuAcGcuGAGuAcuucGAdT^{∧}dT | 89 | UCGAAGuACUcAGCGuAAGdT^{∧}dT | 90 |
| siRNA-008-34 | 19/23 | uGGuAuAuGuGuuGCuGAu | 145 | AUcAGcAAcAcAuAuACcAuu^{∧}a^{∧}a | 146 |

TABLE 19

| | Encapsulation | Average particle | Polydispersity |
|---|---|---|---|
| Double strand ID | efficiency | size (nm) | index |
| Mock | >90% | 92 | 0.06 |
| siRNA-008-34 | >90% | 88 | 0.1 |

### (In-Vivo Evaluation)

LNPs encapsulating PBS or siRNA listed in Table 18 therein were intravenously administered to a BALB/c mouse (male, 6 weeks old, n=3 per group) from the tail vein at a dose of 1 to 3 mg/kg siRNA, and the blood was sampled under anesthesia 5 days and 9 days after the administration. The blood sampled on each sampling day was placed in a blood separator tube containing clot activator (Immuno-Biological Laboratories Co, Ltd., Cat#31203) and centrifuged at 3000 rpm for 15 minutes, and then the serum as the supernatant was collected and stored at −80° C. Thereafter, the complement activity in the serum was quantified in the following manner Specifically, sheep erythrocytes with a concentration of 1.5×10⁸ cells/mL were prepared by using a serum complement titer CH50 kit (DENKA SEIKEN Co., Ltd., Cat#400017) in accordance with a protocol provided by the manufacturer. Subsequently, zymosan (Wako Pure Chemical Industries, Ltd., Cat#263-01491) was prepared so as at a dose of 20 μg/mL with a diluting medium attached to the serum complement titer CH50 kit. The sample serum was diluted 40-fold with the same diluting medium. The sheep erythrocytes, the zymosan, and the diluted serum sample each in a volume of 50 μL were mixed together, and the mixture was incubated at 37° C. overnight. On the next day, the assay plate was centrifuged at 2000 rpm at room temperature for 10 minutes, and the absorbance of the supernatant was then measured at 405 nm. Values for the samples as the complement activity in the serum on the day before the administration to each individual was defined as 100% are shown in Table 20.

TABLE 20

| | | Complement activity | |
|---|---|---|---|
| | | (n = 3, average) | |
| | siRNA | 5 days after | 9 days after |
| Double strand ID | (mg/kg) | administration | administration |
| PBS | — | 106% | Not tested |
| Mock | 1 | 130% | 126% |
| siRNA-008-34 | 1 | 2% | 6% |
| siRNA-008-34 | 3 | 2% | 0% |

### Example 8: Pharmacological Test (Hemolysis-Suppressing Effect with Single Administration)

(Preparation of siRNA-LNPs)

LNPs encapsulating siRNA therein were prepared in the same manner as in Example 3, except that siRNAs listed in Table 21 were used. Results of evaluation of product quality for the prepared LNPs are shown in Table 22.

TABLE 21

| | Numbers of | | | | |
|---|---|---|---|---|---|
| | nucleotides | | | | |
| | in sense | | | | |
| | strand/ | Sense strand | | Antisense strand | |
| Double | antisense | | SEQ | | SEQ |
| strand ID | strand | Sequence (5′→3′) | ID NO | Sequence (5′→3′) | ID NO |
| SiRNA-008-34 | 19/23 | uGGuAuAuGuGuuGCuGAu | 145 | AUcAGcAAcAcAuAuACcAuu^{∧}a^{∧}a | 146 |

TABLE 22

| Double | Encapsulation | Average particle | Polydispersity |
|---|---|---|---|
| strand ID | efficiency | size (nm) | index |
| siRNA-008-34 | >90% | 92 | 0.09 |

### (In-Vivo Evaluation)

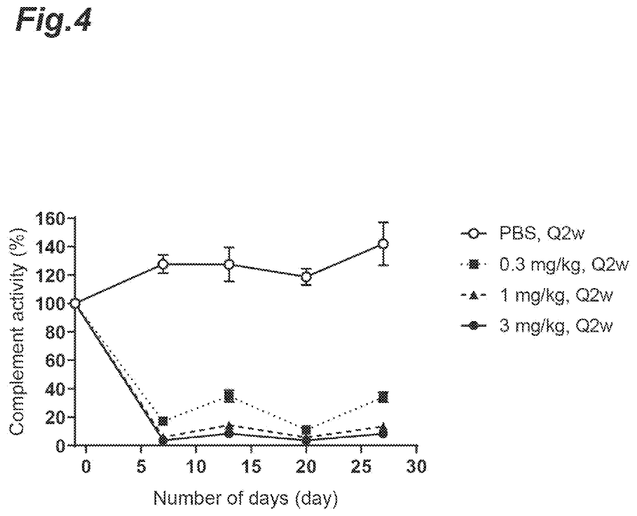

LNPs encapsulating PBS or siRNA listed in Table 21 therein were intravenously administered to a BALB/c mouse (male, 7 weeks old, n=4 per group) from the tail vein at a dose of 0.3, 1 and 3 mg/kg siRNA. The blood was sampled under anesthesia on the day before the administration (−1 Day in Table 23), and 7 days, 13 days, 20 days and 27 days after the administration. The blood sampled on each sampling day was placed in a blood separator tube containing clot activator (Immuno-Biological Laboratories Co, Ltd., Cat#31203) and centrifuged at 3000 rpm for 15 minutes, and then the serum as the supernatant was collected and stored at −80° C. Thereafter, the complement activity in the serum was quantified in the following manner. Specifically, sheep erythrocytes with a concentration of 1.5×10⁸ cells/mL were prepared by using a serum complement titer CH50 kit (DENKA SEIKEN Co., Ltd., Cat#400017) in accordance with a protocol provided by the manufacturer. Subsequently, zymosan (Wako Pure Chemical Industries, Ltd., Cat#263-01491) was prepared so as to achieve a dose of 20 μg/mL with a diluting medium attached to the serum complement titer CH50 kit. The sample serum was diluted 40-fold with the same diluting medium. The sheep erythrocytes, the zymosan, and the diluted serum sample each in a volume of 50 μL were mixed together, and the mixture was incubated at 37° C. overnight. On the next day, the assay plate was centrifuged at 2000 rpm at room temperature for 10 minutes, and the absorbance of the supernatant was then measured at 405 nm. Values for the samples as the complement activity in the serum on the day before the administration to each individual was defined as 100% are shown in Table 23. Complement activity from 1 mouse in 1 mg/kg group was excluded because it was seemed to be outlier. Therefore, the value of 1 mg/kg group in Table 23 is shown as the average of 3 mice. The values of PBS group, 0.3 mg/kg group and 3 mg/kg group in Table 23 are shown as the average of 4 mice. The results are also shown in FIG. 3.

TABLE 23

| Double | siRNA | Complement activity (average) | | | | |
|---|---|---|---|---|---|---|
| strand ID | (mg/kg) | −1 Day | 7 Day | 13 Day | 20 Day | 27 Day |
| PBS | — | 100% | 133% | 122% | 118% | 129% |
| siRNA-008-34 | 0.3 | 100% | 7% | 25% | 55% | 82% |
| siRNA-008-34 | 1 | 100% | 9% | 13% | 29% | 75% |
| siRNA-008-34 | 3 | 100% | 5% | 10% | 26% | 56% |

### Example 9: Pharmacological Test (Hemolysis-Suppressing Effect with Bi-Weekly Administration)

(Preparation of siRNA-LNPs)

LNPs encapsulating siRNA therein were prepared in the same manner as in Example 8.

### (In-Vivo Evaluation)

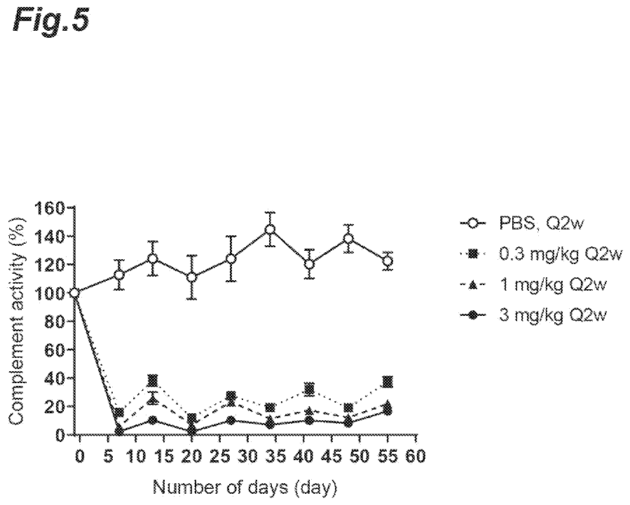

LNPs encapsulating PBS or siRNA listed in Table 21 of Example 8 therein were intravenously administered to a BALB/c mouse (male, 7 weeks old, n=4 per group) from the tail vein at a dose of 0.3, 1 and 3 mg/kg siRNA bi-weekly. The blood was sampled under anesthesia on the day before the administration (−1 Day in Table 24), and 7 days, 13 days, 20 days and 27 days after the administration. The blood sampled on each sampling day was placed in a blood separator tube containing clot activator (Immuno-Biological Laboratories Co, Ltd., Cat#31203) and centrifuged at 3000 rpm for 15 minutes, and then the serum as the supernatant was collected and stored at −80° C. Thereafter, the complement activity in the serum was quantified in the following manner Specifically, sheep erythrocytes with a concentration of 1.5×10⁸ cells/mL were prepared by using a serum complement titer CH50 kit (DENKA SEIKEN Co., Ltd., Cat#400017) in accordance with a protocol provided by the manufacturer. Subsequently, zymosan (Wako Pure Chemical Industries, Ltd., Cat#263-01491) was prepared so as to achieve a dose of 20 μg/mL with a diluting medium attached to the serum complement titer CH50 kit. The sample serum was diluted 40-fold with the same diluting medium. The sheep erythrocytes, the zymosan, and the diluted serum sample each in a volume of 50 μL were mixed together, and the mixture was incubated at 37° C. overnight. On the next day, the assay plate was centrifuged at 2000 rpm at room temperature for 10 minutes, and the absorbance of the supernatant was then measured at 405 nm. Values for the samples as the complement activity in the serum on the day before the administration to each individual was defined as 100% are shown in Table 24. The results are also shown in FIG. 4.

TABLE 24

| | | Complement activity | | | | |
|---|---|---|---|---|---|---|
| Double | siRNA | (n = 4, average) | | | | |
| strand ID | (mg/kg) | −1 Day | 7 Day | 13 Day | 20 Day | 27 Day |
| PBS | — | 100% | 128% | 128% | 119% | 142% |
| siRNA-008-34 | 0.3 | 100% | 17% | 35% | 11% | 34% |
| siRNA-008-34 | 1 | 100% | 6% | 14% | 6% | 13% |
| siRNA-008-34 | 3 | 100% | 4% | 8% | 4% | 8% |

### Example 10: Pharmacological Test (Hemolysis-Suppressing Effect with Bi-Weekly Administration)

(Preparation of siRNA-LNPs)

LNPs encapsulating siRNA therein were prepared in the same manner as in Example 8.

### (In-Vivo Evaluation)

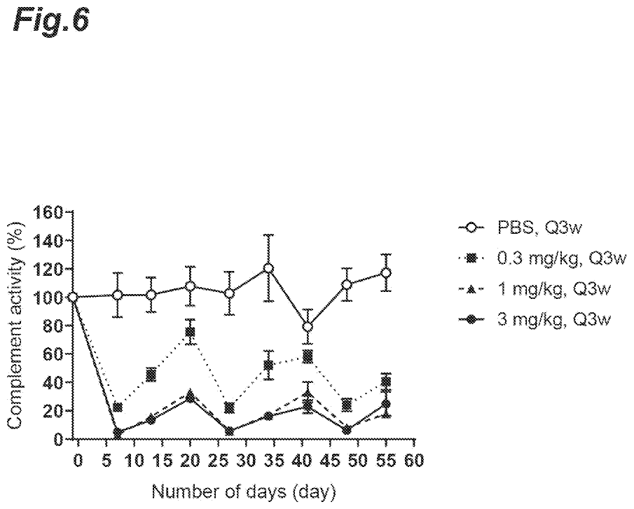

LNPs encapsulating PBS or siRNA listed in Table 21 of Example 8 therein were intravenously administered to a BALB/c mouse (male, 7 weeks old, n=4 per group) from the tail vein at a dose of 0.3, 1 and 3 mg/kg siRNA bi-weekly. The blood was sampled under anesthesia on the day before the administration (−1 Day in Table 25), and 7 days, 13 days, 20 days, 27 days, 34 days, 41 days, 48 days and 55 days after the administration. The blood sampled on each sampling day was placed in a blood separator tube containing clot activator (Immuno-Biological Laboratories Co, Ltd., Cat#31203) and centrifuged at 3000 rpm for 15 minutes, and then the serum as the supernatant was collected and stored at −80° C. Thereafter, the complement activity in the serum was quantified in the following manner. Specifically, sheep erythrocytes with a concentration of 1.5×10⁸ cells/mL were prepared by using a serum complement titer CH50 kit (DENKA SEIKEN Co., Ltd., Cat#400017) in accordance with a protocol provided by the manufacturer. Subsequently, zymosan (Wako Pure Chemical Industries, Ltd., Cat#263-01491) was prepared so as to achieve a dose of 20 μg/mL with a diluting medium attached to the serum complement titer CH50 kit. The sample serum was diluted 40-fold with the same diluting medium. The sheep erythrocytes, the zymosan, and the diluted serum sample each in a volume of 50 μL were mixed together, and the mixture was incubated at 37° C. overnight. On the next day, the assay plate was centrifuged at 2000 rpm at room temperature for 10 minutes, and the absorbance of the supernatant was then measured at 405 nm. Values for the samples as the complement activity in the serum on the day before the administration to each individual was defined as 100% are shown in Table 25. The results are also shown in FIG. 5.

TABLE 25

| | | Complement activity | | | | |
|---|---|---|---|---|---|---|
| Double | siRNA | (n = 4, average) | | | | |
| strand ID | (mg/kg) | −1 Day | 7 Day | 13 Day | 20 Day | 27 Day |
| PBS | — | 100% | 113% | 124% | 111% | 124% |
| siRNA-008-34 | 0.3 | 100% | 16% | 38% | 12% | 27% |
| siRNA-008-34 | 1 | 100% | 6% | 26% | 7% | 23% |
| siRNA-008-34 | 3 | 100% | 2% | 10% | 2% | 10% |
| Double | siRNA | | | | | |
| strand ID | (mg/kg) | 34 Day | 41 Day | 48 Day | 55 Day | |
| PBS | — | 145% | 120% | 138% | 122% | |
| siRNA-008-34 | 0.3 | 19% | 32% | 19% | 37% | |
| siRNA-008-34 | 1 | 11% | 17% | 12% | 22% | |
| siRNA-008-34 | 3 | 7% | 10% | 8% | 17% | |

### Example 11: Pharmacological Test (Hemolysis-Suppressing Effect with Administration Once Every Three Weeks)

(Preparation of siRNA-LNPs)

LNPs encapsulating siRNA therein were prepared in the same manner as in Example 8.

### (In-Vivo Evaluation)

LNPs encapsulating PBS or siRNA listed in Table 21 of Example 8 therein were intravenously administered to a BALB/c mouse (male, 7 weeks old, n=4 per group) from the tail vein at a dose of 0.3, 1 and 3 mg/kg siRNA once every three weeks. The blood was sampled under anesthesia on the day before the administration (−1 Day in Table 26), and 7 days, 13 days, 20 days, 27 days, 34 days, 41 days, 48 days and 55 days after the administration. The blood sampled on each sampling day was placed in a blood separator tube containing clot activator (Immuno-Biological Laboratories Co, Ltd., Cat#31203) and centrifuged at 3000 rpm for 15 minutes, and then the serum as the supernatant was collected and stored at −80° C. Thereafter, the complement activity in the serum was quantified in the following manner. Specifically, sheep erythrocytes with a concentration of 1.5×10⁸ cells/mL were prepared by using a serum complement titer CH50 kit (DENKA SEIKEN Co., Ltd., Cat#400017) in accordance with a protocol provided by the manufacturer. Subsequently, zymosan (Wako Pure Chemical Industries, Ltd., Cat#263-01491) was prepared so as to achieve a dose of 20 μg/mL with a diluting medium attached to the serum complement titer CH50 kit. The sample serum was diluted 40-fold with the same diluting medium. The sheep erythrocytes, the zymosan, and the diluted serum sample each in a volume of 50 μL were mixed together, and the mixture was incubated at 37° C. overnight. On the next day, the assay plate was centrifuged at 2000 rpm at room temperature for 10 minutes, and the absorbance of the supernatant was then measured at 405 nm. Values for the samples as the complement activity in the serum on the day before the administration to each individual was defined as 100% are shown in Table 26. The results are also shown in FIG. 6.

TABLE 26

| | | Complement activity | | | | |
|---|---|---|---|---|---|---|
| Double | siRNA | (n = 4, average) | | | | |
| strand ID | (mg/kg) | −1 Day | 7 Day | 13 Day | 20 Day | 27 Day |
| PBS | — | 100% | 102% | 102% | 108% | 103% |
| siRNA-008-34 | 0.3 | 100% | 22% | 45% | 75% | 22% |
| siRNA-008-34 | 1 | 100% | 3% | 16% | 33% | 6% |
| siRNA-008-34 | 3 | 100% | 5% | 13% | 29% | 6% |
| Double | siRNA | | | | | |
| strand ID | (mg/kg) | 34 Day | 41 Day | 48 Day | 55 Day | |
| PBS | — | 120% | 79% | 109% | 117% | |
| siRNA-008-34 | 0.3 | 52% | 58% | 24% | 40% | |
| siRNA-008-34 | 1 | 17% | 33% | 8% | 18% | |
| siRNA-008-34 | 3 | 16% | 23% | 6% | 25% | |

### Example 12: Physical Properties Test

(Particle Diameter of siRNA-LNPs Under Variation of pH of LNP Solution)

siRNA-008-34 shown in Table 4 was dissolved in 10 mM sodium citrate (pH 4.0) to prepare diluted siRNA solution. Lipid solution was prepared by dissolving 2-{9-oxo-9-[(3-pentyloctyl)oxy]nonyl}dodecyl 1-methylpiperidine-4-carboxylate, DSPC (NIPPON FINE CHEMICAL CO., LTD.), Cholesterol (Dishman Carbogen Amcis Limited), and MPEG2000-DMG (NOF CORPORATION) at a mole ratio of 60/10.5/28/1.5 in ethanol. LNPs were obtained by mixing the diluted siRNA solution and the lipid solution at 3:1 of flow rates. The resulting LNP solution was subjected to substitution with PBS (pH 7.5) in accordance with a conventional method, and then the LNPs were concentrated. Aqueous hydrochloric acid or sodium hydroxide solution was added to the concentrate of the LNPs to adjust to pH 6.0 to 8.5. After the pH adjustment, the LNPs were stored in a cool place.

Results of measurement of the particle sizes of those LNPs by using a particle size analyzer (Malvern Panalytical Ltd., Zetasizer Nano ZS) are shown in Table 27.

TABLE 27

| | | Immediately | | |
|---|---|---|---|---|
| | | after pH | After | After |
| | | adjustment | 18 days | 1 month |
| pH 6.0 | Average particle size (nm) | 91.7 | 95.4 | 96.4 |
| | Polydispersity index | 0.05 | 0.04 | 0.04 |
| pH 6.5 | Average particle size (nm) | 91.0 | 97.6 | 99.3 |
| | Polydispersity index | 0.04 | 0.04 | 0.04 |
| pH 7.0 | Average particle size (nm) | 89.6 | 100.7 | 101.4 |
| | Polydispersity index | 0.05 | 0.05 | 0.02 |
| pH 7.5 | Average particle size (nm) | 89.3 | 90.8 | 88.1 |
| | Polydispersity index | 0.05 | 0.04 | 0.05 |
| pH 8.0 | Average particle size (nm) | 88.1 | 90.5 | 89.4 |
| | Polydispersity index | 0.06 | 0.05 | 0.07 |
| pH 8.5 | Average particle size (nm) | 88.2 | 90.1 | 89.4 |
| | Polydispersity index | 0.05 | 0.05 | 0.06 |

### Example 13: Physical Properties Test

(Particle Diameter of siRNA-LNPs Under Variation of pH of LNP Solution)

siRNA-008-34 shown in Table 4 was dissolved in 10 mM sodium citrate (pH 4.0) to prepare diluted siRNA solution. Lipid solution was prepared by dissolving 2-{9-oxo-9-[(3-pentyloctyl)oxy]nonyl}dodecyl 1-methylpiperidine-4-carboxylate, DSPC (NIPPON FINE CHEMICAL CO., LTD.), Cholesterol (Dishman Carbogen Amcis Limited), and MPEG2000-DMG (NOF CORPORATION) at a mole ratio of 60/10.5/28/1.5 in ethanol. LNPs were obtained by mixing the diluted siRNA solution and the lipid solution at 3:1 of flow rates. The resulting LNP solution was subjected to substitution with PBS (pH 7.5) in accordance with a conventional method, and then the LNPs were concentrated. Aqueous hydrochloric acid or sodium hydroxide solution was added to the concentrate of the LNPs to adjust to pH 6.5 to 8.5. After the pH adjustment, the LNPs were stored in a cool place (5° C.).

Results of measurement of the particle sizes of those LNPs by using a particle size analyzer (NICOMP380) are shown in Table 28. The LNPs at each pH had an appearance of homogeneous solution with opalescence of white or yellowish white color as a feature, and no change was found from immediately after the pH adjustment to after storage for 1 month.

TABLE 28

| | | Immediately | | |
|---|---|---|---|---|
| | | after pH | After | After |
| | | adjustment | 2 weeks | 1 month |
| pH 6.5 | Average particle size (nm) | 93.2 | 93.8 | 94.5 |
| | Polydispersity index | 0.054 | 0.041 | 0.048 |
| pH 7.0 | Average particle size (nm) | 92.9 | 99.2 | 100.5 |
| | Polydispersity index | 0.056 | 0.045 | 0.043 |
| pH 7.5 | Average particle size (nm) | 92.6 | 91.0 | 91.1 |
| | Polydispersity index | 0.050 | 0.056 | 0.058 |
| pH 8.0 | Average particle size (nm) | 92.0 | 91.4 | 91.4 |
| | Polydispersity index | 0.054 | 0.054 | 0.055 |
| pH 8.5※ | Average particle size (nm) | 92.2 | 91.9 | 92.0 |
| | Polydispersity index | 0.055 | 0.049 | 0.049 |

*The pH of the LNP solution was adjusted so as to reach pH 8.5, but the actual measurement immediately after the adjustment was pH 8.3. The pH after storage for 1 month was pH 8.2.

### Example 14: Physical Properties Test

(Particle Diameter of siRNA-LNPs Under Variation of pH of LNP Solution)

siRNA-008-34 shown in Table 4 was dissolved in 10 mM sodium citrate (pH 4.0) to prepare diluted siRNA solution. Lipid solution was prepared by dissolving 2-{9-oxo-9-[(3-pentylo ctyl)oxy]nonyl}dodecyl 1-methylpiperidine-4-carboxylate, DSPC (NIPPON FINE CHEMICAL CO., LTD.), Cholesterol (Dishman Carbogen Amcis Limited), and MPEG2000-DMG (NOF CORPORATION) at a mole ratio of 60/10.5/28/1.5 in ethanol. LNPs were obtained by mixing the diluted siRNA solution and the lipid solution at 3:1 of flow rates. The resulting LNP solution was subjected to substitution with PBS (pH 7.5) in accordance with a conventional method, and then the LNPs were concentrated. Aqueous hydrochloric acid or sodium hydroxide solution was added to the concentrate of the LNPs to adjust to pH 6.5 to 8.5. After the pH adjustment, the LNPs were stored in a cool place (5° C.).

Results of measurement of the particle sizes of those LNPs by using a particle size analyzer (NICOMP380) are shown in Table 29.

TABLE 29

| | | After |
|---|---|---|
| | | 3 months |
| pH 6.5 | Average particle size (nm) | 107.0 |
| | Polydispersity index | 0.048 |
| pH 7.0 | Average particle size (nm) | 103.8 |
| | Polydispersity index | 0.063 |
| pH 7.5 | Average particle size (nm) | 91.8 |
| | Polydispersity index | 0.064 |
| pH 8.0 | Average particle size (nm) | 91.1 |
| | Polydispersity index | 0.061 |
| pH 8.5 | Average particle size (nm) | 91.6 |
| | Polydispersity index | 0.045 |

*1: The pH after storage for 3 months was pH 7.7.
*2: The pH after storage for 3 months was pH 7.9.

### Example 15: Physical Properties Test

(Particle Diameter of siRNA-LNPs Under Variation of pH of LNP Solution)

siRNA-008-34 shown in Table 4 was dissolved in 10 mM sodium citrate (pH 4.0) to prepare diluted siRNA solution. Lipid solution was prepared by dissolving 2-{9-oxo-9-[(3-pentylo ctyl)oxy]nonyl}dodecyl 1-methylpiperidine-4-carboxylate, DSPC (NIPPON FINE CHEMICAL CO., LTD.), Cholesterol (Dishman Carbogen Amcis Limited), and MPEG2000-DMG (NOF CORPORATION) at a mole ratio of 60/10.5/28/1.5 in ethanol. LNPs were obtained by mixing the diluted siRNA solution and the lipid solution at 3:1 of flow rates. The resulting LNP solution was subjected to substitution with PBS (pH 7.5) in accordance with a conventional method, and then the LNPs were concentrated. Aqueous hydrochloric acid or sodium hydroxide solution was added to the concentrate of the LNPs to adjust to pH 6.5 to 8.5. After the pH adjustment, the LNPs were stored at room temperature (25° C.).

Results of measurement of the particle sizes of those LNPs by using a particle size analyzer (NICOMP380) are shown in Table 30.

TABLE 30

| | | Immediately | |
|---|---|---|---|
| | | after pH | After |
| | | adjustment | 2 weeks |
| pH 6.5 | Average particle size (nm) | 93.2 | 95.8 |
| | Polydispersity index | 0.054 | 0.056 |
| pH 7.0 | Average particle size (nm) | 92.9 | 94.5 |
| | Polydispersity index | 0.056 | 0.036 |
| pH 7.5 | Average particle size (nm) | 92.6 | 91.6 |
| | Polydispersity index | 0.050 | 0.056 |
| pH 8.0 | Average particle size (nm) | 92.0 | 91.5 |
| | Polydispersity index | 0.054 | 0.036 |
| pH 8.5※ | Average particle size (nm) | 92.2 | 91.0 |
| | Polydispersity index | 0.055 | 0.048 |

*The pH of the LNP solution was adjusted so as to reach pH 8.5, but the actual measurement immediately after the adjustment was pH 8.3.

### Example 16: Physical Properties Test

(Particle Diameter of siRNA-LNPs under Variation of pH of LNP Solution)

siRNA-008-34 shown in Table 4 was dissolved in 10 mM sodium citrate (pH 4.0) to prepare diluted siRNA solution. Lipid solution was prepared by dissolving 2-{9-oxo-9-[(3-pentylo ctyl)oxy]nonyl}dodecyl 1-methylpiperidine-4-carboxylate, DSPC (Lipoid GmbH), Cholesterol (Dishman Carbogen Amcis Limited), and MPEG2000-DMG (NOF CORPORATION) at a mole ratio of 60/10.5/28/1.5 in ethanol. LNPs were obtained by mixing the diluted siRNA solution and the lipid solution at 3:1 of flow rates. The resulting LNP solution was subjected to substitution with PBS (pH 7.7) in accordance with a conventional method, and then the LNPs were concentrated. Subsequently, the concentrate of the LNPs was subjected to clarifying filtration and concentration adjustment, followed by filtration sterilization. The resulting LNP solution was applied to a dialysis membrane, and dialyzed with Britton-Robinson (BR) buffer solution at pH 2.0/5.0/9.0/10.0/11.0 under room temperature to obtain LNP solutions at different pH values. After the completion of the dialysis, the pH of each LNP solution was confirmed, and the LNP solution was defined as “Immediately after pH adjustment”. After the pH adjustment, the LNP solutions were stored in a cool place (5° C.).

Results of measurement of the particle sizes of those LNPs by using a particle size analyzer (NICOMP380) are shown in Table 31.

TABLE 31

| | | Immediately | | |
|---|---|---|---|---|
| | | after pH | After | After |
| | | adjustment | 2 weeks | 1 month |
| pH 2.0 | Average particle | 94.3 | 94.2 | 94.8 |
| | size (nm) | | | |
| | Polydispersity index | 0.02 | 0.02 | 0.03 |
| PH 5.0 | Average particle | 92.9 | 93.4 | 93.5 |
| | size (nm) | | | |
| | Polydispersity index | 0.02 | 0.03 | 0.02 |
| pH 9.0 | Average particle | 87.8 | 87.2 | 87.2 |
| | size (nm) | | | |
| | Polydispersity index | 0.02 | 0.02 | 0.03 |
| pH 10.0※1 | Average particle | 88.6 | 87.0 | 90.0 |
| | size (nm) | | | |
| | Polydispersity index | 0.04 | 0.03 | 0.03 |
| pH 11.0※2 | Average particle | 90.0 | 88.3 | 89.9 |
| | size (nm) | | | |
| | Polydispersity index | 0.03 | 0.01 | 0.02 |

*1: The pH after storage for 1 month was pH 9.7.
*2: The pH of the LNP solution was adjusted so as to reach pH 11.0, but the actual measurement immediately after the adjustment was pH 10.7. The pH after storage for 2 weeks was pH 10.5, and the pH after storage for 1 month was pH 10.3.

1. A pharmaceutical composition comprising:
a lipid complex,
wherein the lipid complex comprises a double-stranded ribonucleic acid comprising a sense strand consisting of a nucleotide sequence set forth in SEQ ID NO: 145 and an antisense strand consisting of a nucleotide sequence set forth in SEQ ID NO: 146, and
a pH of a solution of the lipid complex is 5.0 or less or 7.5 or more.

2. The pharmaceutical composition according to claim 1, wherein the pH of the solution of the lipid complex is 2.0 or more and 5.0 or less, or 7.5 or more and 11.0 or less.

3. The pharmaceutical composition according to claim 1, wherein the pH of the solution of the lipid complex is 7.5 or more and 10.0 or less.

4. The pharmaceutical composition according to claim 1, wherein an average particle size of the lipid complex is 100 nm or less.

5. The pharmaceutical composition according to claim 1, wherein an average particle size of the lipid complex is 65 nm or more and 100 nm or less.

6. The pharmaceutical composition according to claim 1, wherein an average particle size of the lipid complex is 80 nm or more and 100 nm or less.

7. The pharmaceutical composition according to claim 1, wherein a change in an average particle size of the lipid complex after storage for 2 weeks is 10% or less from an average particle size of the lipid complex before the storage.

8. The pharmaceutical composition according to claim 7, wherein the change in the average particle size of the lipid complex is increase in the average particle size of the lipid complex.

9. The pharmaceutical composition according to claim 1, wherein the lipid complex comprises:
a cationic lipid; and
at least one lipid selected from the group consisting of neutral lipid, polyethylene glycol-modified lipid, and sterol.

10. The pharmaceutical composition according to claim 9, wherein the cationic lipid is 2-{9-oxo-9-[(3-pentyloctyl)oxy]nonyl}dodecyl 1-methylpiperidine-4-carboxylate.

11. The pharmaceutical composition according to claim 1, wherein the lipid complex is a lipid nanoparticle (LNP).

12. The pharmaceutical composition according to claim 1, wherein the lipid complex encapsulates a double-stranded ribonucleic acid comprising a combination of a sense strand and an antisense strand.

13. A method for treating paroxysmal nocturnal hemoglobinuria, comprising administering thepharmaceutical composition according to claim 1 to a patient in need thereof.

14. A method for treating atypical hemolytic uremic syndrome, comprising administering the pharmaceutical composition according to claim 1 to a patient in need thereof.

15. A method for producing the pharmaceutical composition according to claim 1,comprising:
adjusting the pH of the solution of the lipid complex to 5.0 or less, or 7.5 or more.

16. The method according to claim 15, comprising:
adjusting the pH of the solution of the lipid complex to 2.0 or more and 5.0 or less, or 7.5 or more and 11.0 or less.

17. A method for stabilizing the pharmaceutical composition according to claim 1, comprising:
adjusting the pH of the solution of the lipid complex to 5.0 or less, or 7.5 or more.

18. The method according to claim 17, comprising:
adjusting the pH of the solution of the lipid complex to 2.0 or more and 5.0 or less, or 7.5 or more and 11.0 or less.

19. The method according to claim 17, wherein the method for stabilizing the pharmaceutical composition is a method of suppressing a change in an average particle size of the lipid complex in the pharmaceutical composition.

20. The method according to claim 19, wherein the method of suppressing the change in the average particle size is a method of suppressing increase in the average particle size.


SEQUENCE LISTING (172 sequences)

SEQ ID NO: 1 - RNA, 21 nt, Artificial Sequence
  synthesized siRNA sense sequence
  misc_feature (4)..(4): cm
  misc_feature (10)..(10): um
  misc_feature (12)..(13): um
  misc_feature (14)..(14): cm
  misc_feature (20)..(21): n=deoxythymidine
  misc_feature (20)..(21): phosphorothioate bond
aggcaaagguguucaaagann

SEQ ID NO: 2 - RNA, 21 nt, Artificial Sequence
  synthesized siRNA antisense sequence
  misc_feature (9)..(9): cm
  misc_feature (20)..(21): n=deoxythymidine
  misc_feature (20)..(21): phosphorothioate bond
ucuuugaacaccuuugccunn

SEQ ID NO: 3 - RNA, 21 nt, Artificial Sequence
  synthesized siRNA sense sequence
  misc_feature (1)..(1): cm
  misc_feature (2)..(2): um
  misc_feature (4)..(4): um
  misc_feature (5)..(5): cm
  misc_feature (6)..(7): um
  misc_feature (10)..(10): cm
  misc_feature (11)..(13): um
  misc_feature (14)..(14): cm
  misc_feature (16)..(16): um
  misc_feature (20)..(21): n=deoxythymidine
  misc_feature (20)..(21): phosphorothioate bond
cugucuuaacuuucauagann

SEQ ID NO: 4 - RNA, 21 nt, Artificial Sequence
  synthesized siRNA antisense sequence
  misc_feature (3)..(3): um
  misc_feature (12)..(12): um
  misc_feature (17)..(17): cm
  misc_feature (20)..(21): n=deoxythymidine
  misc_feature (20)..(21): phosphorothioate bond
ucuaugaaaguuaagacagnn

SEQ ID NO: 5 - RNA, 21 nt, Artificial Sequence
  synthesized siRNA sense sequence
  misc_feature (1)..(1): um
  misc_feature (4)..(4): cm
  misc_feature (6)..(6): um
  misc_feature (8)..(8): um
  misc_feature (10)..(11): cm
  misc_feature (14)..(14): cm
  misc_feature (15)..(15): um
  misc_feature (17)..(17): cm
  misc_feature (20)..(21): n=deoxythymidine
  misc_feature (20)..(21): phosphorothioate bond
uagcaugugccagcuacaann

SEQ ID NO: 6 - RNA, 21 nt, Artificial Sequence
  synthesized siRNA antisense sequence
  misc_feature (4)..(4): um
  misc_feature (11)..(11): cm
  misc_feature (13)..(13): cm
  misc_feature (18)..(18): um
  misc_feature (20)..(21): n=deoxythymidine
  misc_feature (20)..(21): phosphorothioate bond
uuguagcuggcacaugcuann

SEQ ID NO: 7 - RNA, 21 nt, Artificial Sequence
  synthesized siRNA sense sequence
  misc_feature (1)..(1): cm
  misc_feature (2)..(2): um
  misc_feature (4)..(4): um
  misc_feature (7)..(8): um
  misc_feature (13)..(14): um
  misc_feature (20)..(21): n=deoxythymidine
  misc_feature (20)..(21): phosphorothioate bond
cugugauuggaauuagaaann

SEQ ID NO: 8 - RNA, 21 nt, Artificial Sequence
  synthesized siRNA antisense sequence
  misc_feature (5)..(5): um
  misc_feature (11)..(11): cm
  misc_feature (15)..(15): cm
  misc_feature (17)..(17): cm
  misc_feature (20)..(21): n=deoxythymidine
  misc_feature (20)..(21): phosphorothioate bond
uuucuaauuccaaucacagnn

SEQ ID NO: 9 - RNA, 21 nt, Artificial Sequence
  synthesized siRNA sense sequence
  misc_feature (5)..(5): cm
  misc_feature (11)..(11): um
  misc_feature (13)..(14): um
  misc_feature (15)..(15): cm
  misc_feature (20)..(21): n=deoxythymidine
  misc_feature (20)..(21): phosphorothioate bond
aaggcaaagguguucaaagnn

SEQ ID NO: 10 - RNA, 21 nt, Artificial Sequence
  synthesized siRNA antisense sequence
  misc_feature (8)..(8): cm
  misc_feature (20)..(21): n=deoxythymidine
  misc_feature (20)..(21): phosphorothioate bond
cuuugaacaccuuugccuunn

SEQ ID NO: 11 - RNA, 21 nt, Artificial Sequence
  synthesized siRNA sense sequence
  misc_feature (9)..(9): cm
  misc_feature (10)..(10): um
  misc_feature (12)..(14): um
  misc_feature (16)..(16): cm
  misc_feature (19)..(19): cm
  misc_feature (20)..(21): n=deoxythymidine
  misc_feature (20)..(21): phosphorothioate bond
gaaaggaacuguuuacaacnn

SEQ ID NO: 12 - RNA, 21 nt, Artificial Sequence
  synthesized siRNA antisense sequence
  misc_feature (5)..(5): um
  misc_feature (9)..(9): cm
  misc_feature (20)..(21): n=deoxythymidine
  misc_feature (20)..(21): phosphorothioate bond
guuguaaacaguuccuuucnn

SEQ ID NO: 13 - RNA, 21 nt, Artificial Sequence
  synthesized siRNA sense sequence
  misc_feature (1)..(1): um
  misc_feature (4)..(4): um
  misc_feature (6)..(6): um
  misc_feature (8)..(8): um
  misc_feature (10)..(10): um
  misc_feature (12)..(13): um
  misc_feature (15)..(15): cm
  misc_feature (16)..(16): um
  misc_feature (19)..(19): um
  misc_feature (20)..(21): n=deoxythymidine
  misc_feature (20)..(21): phosphorothioate bond
ugguauauguguugcugaunn

SEQ ID NO: 14 - RNA, 21 nt, Artificial Sequence
  synthesized siRNA antisense sequence
  misc_feature (3)..(3): cm
  misc_feature (6)..(6): cm
  misc_feature (9)..(9): cm
  misc_feature (11)..(11): cm
  misc_feature (13)..(13): um
  misc_feature (15)..(15): um
  misc_feature (18)..(18): cm
  misc_feature (20)..(21): n=deoxythymidine
  misc_feature (20)..(21): phosphorothioate bond
aucagcaacacauauaccann

SEQ ID NO: 15 - RNA, 21 nt, Artificial Sequence
  synthesized siRNA sense sequence
  misc_feature (2)..(2): cm
  misc_feature (3)..(3): um
  misc_feature (5)..(5): um
  misc_feature (6)..(6): cm
  misc_feature (7)..(8): um
  misc_feature (11)..(11): cm
  misc_feature (12)..(14): um
  misc_feature (15)..(15): cm
  misc_feature (17)..(17): um
  misc_feature (20)..(21): n=deoxythymidine
  misc_feature (20)..(21): phosphorothioate bond
acugucuuaacuuucauagnn

SEQ ID NO: 16 - RNA, 21 nt, Artificial Sequence
  synthesized siRNA antisense sequence
  misc_feature (2)..(2): um
  misc_feature (11)..(11): um
  misc_feature (16)..(16): cm
  misc_feature (20)..(21): n=deoxythymidine
  misc_feature (20)..(21): phosphorothioate bond
cuaugaaaguuaagacagunn

SEQ ID NO: 17 - RNA, 21 nt, Artificial Sequence
  synthesized siRNA sense sequence
  misc_feature (2)..(2): um
  misc_feature (4)..(5): cm
  misc_feature (8)..(8): cm
  misc_feature (9)..(9): um
  misc_feature (11)..(11): cm
  misc_feature (15)..(17): cm
  misc_feature (20)..(21): n=deoxythymidine
  misc_feature (20)..(21): phosphorothioate bond
gugccagcuacaagcccagnn

SEQ ID NO: 18 - RNA, 21 nt, Artificial Sequence
  synthesized siRNA antisense sequence
  misc_feature (10)..(10): um
  misc_feature (17)..(17): cm
  misc_feature (20)..(21): n=deoxythymidine
  misc_feature (20)..(21): phosphorothioate bond
cugggcuuguagcuggcacnn

SEQ ID NO: 19 - RNA, 21 nt, Artificial Sequence
  synthesized siRNA sense sequence
  misc_feature (7)..(7): cm
  misc_feature (8)..(8): um
  misc_feature (10)..(12): um
  misc_feature (14)..(14): cm
  misc_feature (17)..(17): cm
  misc_feature (18)..(18): um
  misc_feature (20)..(21): n=deoxythymidine
  misc_feature (20)..(21): phosphorothioate bond
aaggaacuguuuacaacuann

SEQ ID NO: 20 - RNA, 21 nt, Artificial Sequence
  synthesized siRNA antisense sequence
  misc_feature (1)..(1): um
  misc_feature (7)..(7): um
  misc_feature (11)..(11): cm
  misc_feature (20)..(21): n=deoxythymidine
  misc_feature (20)..(21): phosphorothioate bond
uaguuguaaacaguuccuunn

SEQ ID NO: 21 - RNA, 21 nt, Artificial Sequence
  synthesized siRNA sense sequence
  misc_feature (1)..(1): um
  misc_feature (2)..(3): cm
  misc_feature (4)..(4): um
  misc_feature (5)..(5): cm
  misc_feature (6)..(6): um
  misc_feature (12)..(13): um
  misc_feature (16)..(17): cm
  misc_feature (18)..(19): um
  misc_feature (20)..(21): n=deoxythymidine
  misc_feature (20)..(21): phosphorothioate bond
uccucuggaaauuggccuunn

SEQ ID NO: 22 - RNA, 21 nt, Artificial Sequence
  synthesized siRNA antisense sequence
  misc_feature (6)..(6): cm
  misc_feature (13)..(13): cm
  misc_feature (20)..(21): n=deoxythymidine
  misc_feature (20)..(21): phosphorothioate bond
aaggccaauuuccagaggann

SEQ ID NO: 23 - RNA, 21 nt, Artificial Sequence
  synthesized siRNA sense sequence
  misc_feature (1)..(2): um
  misc_feature (11)..(11): cm
  misc_feature (12)..(12): um
  misc_feature (14)..(16): um
  misc_feature (18)..(18): cm
  misc_feature (20)..(21): n=deoxythymidine
  misc_feature (20)..(21): phosphorothioate bond
uugaaaggaacuguuuacann

SEQ ID NO: 24 - RNA, 21 nt, Artificial Sequence
  synthesized siRNA antisense sequence
  misc_feature (3)..(3): um
  misc_feature (7)..(7): cm
  misc_feature (17)..(17): cm
  misc_feature (20)..(21): n=deoxythymidine
  misc_feature (20)..(21): phosphorothioate bond
uguaaacaguuccuuucaann

SEQ ID NO: 25 - RNA, 21 nt, Artificial Sequence
  synthesized siRNA sense sequence
  misc_feature (8)..(8): cm
  misc_feature (9)..(9): um
  misc_feature (11)..(13): um
  misc_feature (15)..(15): cm
  misc_feature (18)..(18): cm
  misc_feature (19)..(19): um
  misc_feature (20)..(21): n=deoxythymidine
  misc_feature (20)..(21): phosphorothioate bond
aaaggaacuguuuacaacunn

SEQ ID NO: 26 - RNA, 21 nt, Artificial Sequence
  synthesized siRNA antisense sequence
  misc_feature (6)..(6): um
  misc_feature (10)..(10): cm
  misc_feature (20)..(21): n=deoxythymidine
  misc_feature (20)..(21): phosphorothioate bond
aguuguaaacaguuccuuunn

SEQ ID NO: 27 - RNA, 21 nt, Artificial Sequence
  synthesized siRNA sense sequence
  misc_feature (6)..(6): cm
  misc_feature (7)..(7): um
  misc_feature (9)..(11): um
  misc_feature (13)..(13): cm
  misc_feature (16)..(16): cm
  misc_feature (17)..(17): um
  misc_feature (19)..(19): um
  misc_feature (20)..(21): n=deoxythymidine
  misc_feature (20)..(21): phosphorothioate bond
aggaacuguuuacaacuaunn

SEQ ID NO: 28 - RNA, 21 nt, Artificial Sequence
  synthesized siRNA antisense sequence
  misc_feature (2)..(2): um
  misc_feature (8)..(8): um
  misc_feature (12)..(12): cm
  misc_feature (20)..(21): n=deoxythymidine
  misc_feature (20)..(21): phosphorothioate bond
auaguuguaaacaguuccunn

SEQ ID NO: 29 - RNA, 21 nt, Artificial Sequence
  synthesized siRNA sense sequence
  misc_feature (1)..(1): um
  misc_feature (3)..(3): cm
  misc_feature (5)..(5): cm
  misc_feature (6)..(6): um
  misc_feature (11)..(11): cm
  misc_feature (13)..(15): um
  misc_feature (18)..(18): um
  misc_feature (20)..(21): n=deoxythymidine
  misc_feature (20)..(21): phosphorothioate bond
uacacugaagcauuugaugnn

SEQ ID NO: 30 - RNA, 21 nt, Artificial Sequence
  synthesized siRNA antisense sequence
  misc_feature (1)..(1): cm
  misc_feature (4)..(4): cm
  misc_feature (13)..(13): cm
  misc_feature (18)..(18): um
  misc_feature (20)..(21): n=deoxythymidine
  misc_feature (20)..(21): phosphorothioate bond
caucaaaugcuucaguguann

SEQ ID NO: 31 - RNA, 21 nt, Artificial Sequence
  synthesized siRNA sense sequence
  misc_feature (1)..(1): cm
  misc_feature (3)..(3): cm
  misc_feature (4)..(4): um
  misc_feature (9)..(9): cm
  misc_feature (11)..(13): um
  misc_feature (16)..(16): um
  misc_feature (18)..(18): cm
  misc_feature (20)..(21): n=deoxythymidine
  misc_feature (20)..(21): phosphorothioate bond
cacugaagcauuugaugcann

SEQ ID NO: 32 - RNA, 21 nt, Artificial Sequence
  synthesized siRNA antisense sequence
  misc_feature (3)..(3): cm
  misc_feature (6)..(6): cm
  misc_feature (15)..(15): cm
  misc_feature (20)..(21): n=deoxythymidine
  misc_feature (20)..(21): phosphorothioate bond
ugcaucaaaugcuucagugnn

SEQ ID NO: 33 - RNA, 21 nt, Artificial Sequence
  synthesized siRNA sense sequence
  misc_feature (1)..(1): cm
  misc_feature (2)..(2): um
  misc_feature (7)..(7): cm
  misc_feature (9)..(11): um
  misc_feature (14)..(14): um
  misc_feature (16)..(16): cm
  misc_feature (19)..(19): cm
  misc_feature (20)..(21): n=deoxythymidine
  misc_feature (20)..(21): phosphorothioate bond
cugaagcauuugaugcaacnn

SEQ ID NO: 34 - RNA, 21 nt, Artificial Sequence
  synthesized siRNA antisense sequence
  misc_feature (5)..(5): cm
  misc_feature (8)..(8): cm
  misc_feature (17)..(17): cm
  misc_feature (20)..(21): n=deoxythymidine
  misc_feature (20)..(21): phosphorothioate bond
guugcaucaaaugcuucagnn

SEQ ID NO: 35 - RNA, 21 nt, Artificial Sequence
  synthesized siRNA sense sequence
  misc_feature (1)..(2): um
  misc_feature (3)..(3): cm
  misc_feature (4)..(4): um
  misc_feature (6)..(6): cm
  misc_feature (9)..(9): cm
  misc_feature (10)..(10): um
  misc_feature (14)..(15): um
  misc_feature (16)..(16): cm
  misc_feature (19)..(19): um
  misc_feature (20)..(21): n=deoxythymidine
  misc_feature (20)..(21): phosphorothioate bond
uucugcaacugaauucgaunn

SEQ ID NO: 36 - RNA, 21 nt, Artificial Sequence
  synthesized siRNA antisense sequence
  misc_feature (9)..(9): cm
  misc_feature (15)..(15): cm
  misc_feature (20)..(21): n=deoxythymidine
  misc_feature (20)..(21): phosphorothioate bond
aucgaauucaguugcagaann

SEQ ID NO: 37 - RNA, 21 nt, Artificial Sequence
  synthesized siRNA sense sequence
  misc_feature (1)..(1): um
  misc_feature (10)..(10): cm
  misc_feature (11)..(11): um
  misc_feature (13)..(15): um
  misc_feature (17)..(17): cm
  misc_feature (20)..(21): n=deoxythymidine
  misc_feature (20)..(21): phosphorothioate bond
ugaaaggaacuguuuacaann

SEQ ID NO: 38 - RNA, 21 nt, Artificial Sequence
  synthesized siRNA antisense sequence
  misc_feature (4)..(4): cm
  misc_feature (8)..(8): cm
  misc_feature (18)..(18): cm
  misc_feature (20)..(21): n=deoxythymidine
  misc_feature (20)..(21): phosphorothioate bond
uuguaaacaguuccuuucann

SEQ ID NO: 39 - RNA, 21 nt, Artificial Sequence
  synthesized siRNA sense sequence
  misc_feature (2)..(2): cm
  misc_feature (3)..(3): um
  misc_feature (8)..(8): cm
  misc_feature (10)..(12): um
  misc_feature (15)..(15): um
  misc_feature (17)..(17): cm
  misc_feature (20)..(21): n=deoxythymidine
  misc_feature (20)..(21): phosphorothioate bond
acugaagcauuugaugcaann

SEQ ID NO: 40 - RNA, 21 nt, Artificial Sequence
  synthesized siRNA antisense sequence
  misc_feature (4)..(4): cm
  misc_feature (7)..(7): cm
  misc_feature (16)..(16): cm
  misc_feature (20)..(21): n=deoxythymidine
  misc_feature (20)..(21): phosphorothioate bond
uugcaucaaaugcuucagunn

SEQ ID NO: 41 - RNA, 21 nt, Artificial Sequence
  synthesized siRNA sense sequence
  misc_feature (1)..(1): cm
  misc_feature (3)..(3): um
  misc_feature (5)..(5): cm
  misc_feature (9)..(9): cm
  misc_feature (13)..(14): cm
  misc_feature (15)..(15): um
  misc_feature (17)..(19): um
  misc_feature (20)..(21): n=deoxythymidine
  misc_feature (20)..(21): phosphorothioate bond
cauacagacaaaccuguuunn

SEQ ID NO: 42 - RNA, 21 nt, Artificial Sequence
  synthesized siRNA antisense sequence
  misc_feature (4)..(4): cm
  misc_feature (16)..(16): um
  misc_feature (20)..(21): n=deoxythymidine
  misc_feature (20)..(21): phosphorothioate bond
aaacagguuugucuguaugnn

SEQ ID NO: 43 - RNA, 21 nt, Artificial Sequence
  synthesized siRNA sense sequence
  misc_feature (4)..(4): cm
  misc_feature (7)..(7): cm
  misc_feature (11)..(11): um
  misc_feature (13)..(14): cm
  misc_feature (15)..(17): um
  misc_feature (19)..(19): um
  misc_feature (20)..(21): n=deoxythymidine
  misc_feature (20)..(21): phosphorothioate bond
aaacaacaaguaccuuuaunn

SEQ ID NO: 44 - RNA, 21 nt, Artificial Sequence
  synthesized siRNA antisense sequence
  misc_feature (2)..(2): um
  misc_feature (8)..(8): um
  misc_feature (20)..(21): n=deoxythymidine
  misc_feature (20)..(21): phosphorothioate bond
auaaagguacuuguuguuunn

SEQ ID NO: 45 - RNA, 21 nt, Artificial Sequence
  synthesized siRNA sense sequence
  misc_feature (2)..(2): um
  misc_feature (4)..(4): cm
  misc_feature (8)..(8): cm
  misc_feature (12)..(13): cm
  misc_feature (14)..(14): um
  misc_feature (16)..(18): um
  misc_feature (20)..(21): n=deoxythymidine
  misc_feature (20)..(21): phosphorothioate bond
auacagacaaaccuguuuann

SEQ ID NO: 46 - RNA, 21 nt, Artificial Sequence
  synthesized siRNA antisense sequence
  misc_feature (1)..(1): um
  misc_feature (5)..(5): cm
  misc_feature (17)..(17): um
  misc_feature (20)..(21): n=deoxythymidine
  misc_feature (20)..(21): phosphorothioate bond
uaaacagguuugucuguaunn

SEQ ID NO: 47 - RNA, 21 nt, Artificial Sequence
  synthesized siRNA sense sequence
  misc_feature (3)..(3): um
  misc_feature (5)..(5): um
  misc_feature (7)..(7): um
  misc_feature (9)..(9): um
  misc_feature (11)..(12): um
  misc_feature (14)..(14): cm
  misc_feature (15)..(15): um
  misc_feature (18)..(18): um
  misc_feature (20)..(21): n=deoxythymidine
  misc_feature (20)..(21): phosphorothioate bond
gguauauguguugcugauann

SEQ ID NO: 48 - RNA, 21 nt, Artificial Sequence
  synthesized siRNA antisense sequence
  misc_feature (1)..(1): um
  misc_feature (4)..(4): cm
  misc_feature (7)..(7): cm
  misc_feature (10)..(10): cm
  misc_feature (12)..(12): cm
  misc_feature (14)..(14): um
  misc_feature (16)..(16): um
  misc_feature (20)..(21): n=deoxythymidine
  misc_feature (20)..(21): phosphorothioate bond
uaucagcaacacauauaccnn

SEQ ID NO: 49 - RNA, 21 nt, Artificial Sequence
  synthesized siRNA sense sequence
  misc_feature (1)..(1): um
  misc_feature (2)..(2): cm
  misc_feature (9)..(9): um
  misc_feature (10)..(10): cm
  misc_feature (11)..(11): um
  misc_feature (13)..(13): um
  misc_feature (20)..(21): n=deoxythymidine
  misc_feature (20)..(21): phosphorothioate bond
ucagaaagucugugaaggann

SEQ ID NO: 50 - RNA, 21 nt, Artificial Sequence
  synthesized siRNA antisense sequence
  misc_feature (6)..(6): cm
  misc_feature (8)..(8): cm
  misc_feature (20)..(21): n=deoxythymidine
  misc_feature (20)..(21): phosphorothioate bond
uccuucacagacuuucugann

SEQ ID NO: 51 - RNA, 21 nt, Artificial Sequence
  synthesized siRNA sense sequence
  misc_feature (1)..(1): um
  misc_feature (2)..(2): cm
  misc_feature (3)..(3): um
  misc_feature (4)..(5): cm
  misc_feature (9)..(10): cm
  misc_feature (14)..(14): cm
  misc_feature (15)..(15): um
  misc_feature (17)..(17): um
  misc_feature (19)..(19): um
  misc_feature (20)..(21): n=deoxythymidine
  misc_feature (20)..(21): phosphorothioate bond
ucuccaggccaaacugugunn

SEQ ID NO: 52 - RNA, 21 nt, Artificial Sequence
  synthesized siRNA antisense sequence
  misc_feature (2)..(2): cm
  misc_feature (4)..(4): cm
  misc_feature (20)..(21): n=deoxythymidine
  misc_feature (20)..(21): phosphorothioate bond
acacaguuuggccuggagann

SEQ ID NO: 53 - RNA, 21 nt, Artificial Sequence
  synthesized siRNA sense sequence
  misc_feature (2)..(2): cm
  misc_feature (5)..(5): cm
  misc_feature (9)..(9): um
  misc_feature (11)..(12): cm
  misc_feature (13)..(15): um
  misc_feature (17)..(17): um
  misc_feature (19)..(19): um
  misc_feature (20)..(21): n=deoxythymidine
  misc_feature (20)..(21): phosphorothioate bond
acaacaaguaccuuuauaunn

SEQ ID NO: 54 - RNA, 21 nt, Artificial Sequence
  synthesized siRNA antisense sequence
  misc_feature (2)..(2): um
  misc_feature (4)..(4): um
  misc_feature (10)..(10): um
  misc_feature (20)..(21): n=deoxythymidine
  misc_feature (20)..(21): phosphorothioate bond
auauaaagguacuuguugunn

SEQ ID NO: 55 - RNA, 21 nt, Artificial Sequence
  synthesized siRNA sense sequence
  misc_feature (1)..(1): cm
  misc_feature (4)..(4): cm
  misc_feature (8)..(8): um
  misc_feature (10)..(11): cm
  misc_feature (12)..(14): um
  misc_feature (16)..(16): um
  misc_feature (18)..(19): um
  misc_feature (20)..(21): n=deoxythymidine
  misc_feature (20)..(21): phosphorothioate bond
caacaaguaccuuuauauunn

SEQ ID NO: 56 - RNA, 21 nt, Artificial Sequence
  synthesized siRNA antisense sequence
  misc_feature (3)..(3): um
  misc_feature (5)..(5): um
  misc_feature (11)..(11): um
  misc_feature (20)..(21): n=deoxythymidine
  misc_feature (20)..(21): phosphorothioate bond
aauauaaagguacuuguugnn

SEQ ID NO: 57 - RNA, 21 nt, Artificial Sequence
  synthesized siRNA sense sequence
  misc_feature (2)..(3): um
  misc_feature (4)..(4): cm
  misc_feature (5)..(5): um
  misc_feature (6)..(7): cm
  misc_feature (11)..(12): cm
  misc_feature (16)..(16): cm
  misc_feature (17)..(17): um
  misc_feature (19)..(19): um
  misc_feature (20)..(21): n=deoxythymidine
  misc_feature (20)..(21): phosphorothioate bond
auucuccaggccaaacugunn

SEQ ID NO: 58 - RNA, 21 nt, Artificial Sequence
  synthesized siRNA antisense sequence
  misc_feature (2)..(2): cm
  misc_feature (20)..(21): n=deoxythymidine
  misc_feature (20)..(21): phosphorothioate bond
acaguuuggccuggagaaunn

SEQ ID NO: 59 - RNA, 21 nt, Artificial Sequence
  synthesized siRNA sense sequence
  misc_feature (2)..(2): um
  misc_feature (5)..(5): cm
  misc_feature (8)..(9): cm
  misc_feature (12)..(12): cm
  misc_feature (13)..(13): um
  misc_feature (14)..(15): cm
  misc_feature (19)..(19): um
  misc_feature (20)..(21): n=deoxythymidine
  misc_feature (20)..(21): phosphorothioate bond
guggcaaccagcuccaggunn

SEQ ID NO: 60 - RNA, 21 nt, Artificial Sequence
  synthesized siRNA antisense sequence
  misc_feature (17)..(17): cm
  misc_feature (20)..(21): n=deoxythymidine
  misc_feature (20)..(21): phosphorothioate bond
accuggagcugguugccacnn

SEQ ID NO: 61 - RNA, 21 nt, Artificial Sequence
  synthesized siRNA sense sequence
  misc_feature (7)..(7): cm
  misc_feature (9)..(9): um
  misc_feature (10)..(10): cm
  misc_feature (11)..(11): um
  misc_feature (14)..(14): cm
  misc_feature (15)..(16): um
  misc_feature (20)..(21): n=deoxythymidine
  misc_feature (20)..(21): phosphorothioate bond
aagagacaucugacuuggann

SEQ ID NO: 62 - RNA, 21 nt, Artificial Sequence
  synthesized siRNA antisense sequence
  misc_feature (3)..(3): cm
  misc_feature (8)..(8): cm
  misc_feature (20)..(21): n=deoxythymidine
  misc_feature (20)..(21): phosphorothioate bond
uccaagucagaugucucuunn

SEQ ID NO: 63 - RNA, 21 nt, Artificial Sequence
  synthesized siRNA sense sequence
  misc_feature (2)..(3): um
  misc_feature (4)..(4): cm
  misc_feature (5)..(5): um
  misc_feature (7)..(7): cm
  misc_feature (10)..(10): cm
  misc_feature (11)..(11): um
  misc_feature (15)..(16): um
  misc_feature (17)..(17): cm
  misc_feature (20)..(21): n=deoxythymidine
  misc_feature (20)..(21): phosphorothioate bond
auucugcaacugaauucgann

SEQ ID NO: 64 - RNA, 21 nt, Artificial Sequence
  synthesized siRNA antisense sequence
  misc_feature (8)..(8): cm
  misc_feature (14)..(14): cm
  misc_feature (20)..(21): n=deoxythymidine
  misc_feature (20)..(21): phosphorothioate bond
ucgaauucaguugcagaaunn

SEQ ID NO: 65 - RNA, 21 nt, Artificial Sequence
  synthesized siRNA sense sequence
  misc_feature (1)..(2): um
  misc_feature (3)..(4): cm
  misc_feature (5)..(5): um
  misc_feature (6)..(6): cm
  misc_feature (7)..(7): um
  misc_feature (13)..(14): um
  misc_feature (17)..(18): cm
  misc_feature (19)..(19): um
  misc_feature (20)..(21): n=deoxythymidine
  misc_feature (20)..(21): phosphorothioate bond
uuccucuggaaauuggccunn

SEQ ID NO: 66 - RNA, 21 nt, Artificial Sequence
  synthesized siRNA antisense sequence
  misc_feature (5)..(5): cm
  misc_feature (12)..(12): cm
  misc_feature (20)..(21): n=deoxythymidine
  misc_feature (20)..(21): phosphorothioate bond
aggccaauuuccagaggaann

SEQ ID NO: 67 - RNA, 21 nt, Artificial Sequence
  synthesized siRNA sense sequence
  misc_feature (3)..(3): cm
  misc_feature (6)..(6): cm
  misc_feature (10)..(10): um
  misc_feature (12)..(13): cm
  misc_feature (14)..(16): um
  misc_feature (18)..(18): um
  misc_feature (20)..(21): n=deoxythymidine
  misc_feature (20)..(21): phosphorothioate bond
aacaacaaguaccuuuauann

SEQ ID NO: 68 - RNA, 21 nt, Artificial Sequence
  synthesized siRNA antisense sequence
  misc_feature (1)..(1): um
  misc_feature (3)..(3): um
  misc_feature (9)..(9): um
  misc_feature (20)..(21): n=deoxythymidine
  misc_feature (20)..(21): phosphorothioate bond
uauaaagguacuuguuguunn

SEQ ID NO: 69 - RNA, 21 nt, Artificial Sequence
  synthesized siRNA sense sequence
  misc_feature (3)..(3): um
  misc_feature (5)..(5): um
  misc_feature (7)..(7): um
  misc_feature (8)..(9): cm
  misc_feature (10)..(10): um
  misc_feature (11)..(11): cm
  misc_feature (12)..(12): um
  misc_feature (13)..(13): cm
  misc_feature (14)..(14): um
  misc_feature (15)..(17): cm
  misc_feature (18)..(18): um
  misc_feature (20)..(21): n=deoxythymidine
  misc_feature (20)..(21): phosphorothioate bond
aauauguccucucucccuann

SEQ ID NO: 70 - RNA, 21 nt, Artificial Sequence
  synthesized siRNA antisense sequence
  misc_feature (1)..(1): um
  misc_feature (14)..(14): cm
  misc_feature (16)..(16): um
  misc_feature (20)..(21): n=deoxythymidine
  misc_feature (20)..(21): phosphorothioate bond
uagggagagaggacauauunn

SEQ ID NO: 71 - RNA, 21 nt, Artificial Sequence
  synthesized siRNA sense sequence
  misc_feature (2)..(2): cm
  misc_feature (3)..(3): um
  misc_feature (4)..(4): cm
  misc_feature (6)..(6): cm
  misc_feature (7)..(7): um
  misc_feature (9)..(9): um
  misc_feature (12)..(13): um
  misc_feature (15)..(15): cm
  misc_feature (16)..(17): um
  misc_feature (20)..(21): n=deoxythymidine
  misc_feature (20)..(21): phosphorothioate bond
acucacuauaauuacuugann

SEQ ID NO: 72 - RNA, 21 nt, Artificial Sequence
  synthesized siRNA antisense sequence
  misc_feature (2)..(2): cm
  misc_feature (6)..(6): um
  misc_feature (10)..(10): um
  misc_feature (12)..(12): um
  misc_feature (20)..(21): n=deoxythymidine
  misc_feature (20)..(21): phosphorothioate bond
ucaaguaauuauagugagunn

SEQ ID NO: 73 - RNA, 21 nt, Artificial Sequence
  synthesized siRNA sense sequence
  misc_feature (2)..(2): um
  misc_feature (5)..(5): cm
  misc_feature (6)..(6): um
  misc_feature (7)..(7): cm
  misc_feature (9)..(9): cm
  misc_feature (10)..(10): um
  misc_feature (12)..(12): um
  misc_feature (15)..(16): um
  misc_feature (18)..(18): cm
  misc_feature (19)..(19): um
  misc_feature (20)..(21): n=deoxythymidine
  misc_feature (20)..(21): phosphorothioate bond
auaacucacuauaauuacunn

SEQ ID NO: 74 - RNA, 21 nt, Artificial Sequence
  synthesized siRNA antisense sequence
  misc_feature (3)..(3): um
  misc_feature (7)..(7): um
  misc_feature (9)..(9): um
  misc_feature (17)..(17): um
  misc_feature (20)..(21): n=deoxythymidine
  misc_feature (20)..(21): phosphorothioate bond
aguaauuauagugaguuaunn

SEQ ID NO: 75 - RNA, 21 nt, Artificial Sequence
  synthesized siRNA sense sequence
  misc_feature (4)..(4): um
  misc_feature (6)..(6): um
  misc_feature (8)..(8): um
  misc_feature (9)..(10): cm
  misc_feature (11)..(11): um
  misc_feature (12)..(12): cm
  misc_feature (13)..(13): um
  misc_feature (14)..(14): cm
  misc_feature (15)..(15): um
  misc_feature (16)..(18): cm
  misc_feature (19)..(19): um
  misc_feature (20)..(21): n=deoxythymidine
  misc_feature (20)..(21): phosphorothioate bond
aaauauguccucucucccunn

SEQ ID NO: 76 - RNA, 21 nt, Artificial Sequence
  synthesized siRNA antisense sequence
  misc_feature (13)..(13): cm
  misc_feature (15)..(15): um
  misc_feature (20)..(21): n=deoxythymidine
  misc_feature (20)..(21): phosphorothioate bond
agggagagaggacauauuunn

SEQ ID NO: 77 - RNA, 21 nt, Artificial Sequence
  synthesized siRNA sense sequence
  misc_feature (5)..(5): um
  misc_feature (7)..(11): um
  misc_feature (13)..(13): um
  misc_feature (16)..(16): um
  misc_feature (20)..(21): n=deoxythymidine
  misc_feature (20)..(21): phosphorothioate bond
aagauauuuuuauaauaaann

SEQ ID NO: 78 - RNA, 21 nt, Artificial Sequence
  synthesized siRNA antisense sequence
  misc_feature (3)..(3): um
  misc_feature (6)..(6): um
  misc_feature (8)..(8): um
  misc_feature (14)..(14): um
  misc_feature (20)..(21): n=deoxythymidine
  misc_feature (20)..(21): phosphorothioate bond
uuuauuauaaaaauaucuunn

SEQ ID NO: 79 - RNA, 21 nt, Artificial Sequence
  synthesized siRNA sense sequence
  misc_feature (5)..(5): um
  misc_feature (8)..(8): cm
  misc_feature (9)..(9): um
  misc_feature (10)..(10): cm
  misc_feature (12)..(12): cm
  misc_feature (13)..(13): um
  misc_feature (15)..(15): um
  misc_feature (18)..(19): um
  misc_feature (20)..(21): n=deoxythymidine
  misc_feature (20)..(21): phosphorothioate bond
aaaauaacucacuauaauunn

SEQ ID NO: 80 - RNA, 21 nt, Artificial Sequence
  synthesized siRNA antisense sequence
  misc_feature (4)..(4): um
  misc_feature (6)..(6): um
  misc_feature (14)..(14): um
  misc_feature (20)..(21): n=deoxythymidine
  misc_feature (20)..(21): phosphorothioate bond
aauuauagugaguuauuuunn

SEQ ID NO: 81 - RNA, 21 nt, Artificial Sequence
  synthesized siRNA sense sequence
  misc_feature (4)..(4): um
  misc_feature (7)..(7): cm
  misc_feature (8)..(8): um
  misc_feature (9)..(9): cm
  misc_feature (11)..(11): cm
  misc_feature (12)..(12): um
  misc_feature (14)..(14): um
  misc_feature (17)..(18): um
  misc_feature (20)..(21): n=deoxythymidine
  misc_feature (20)..(21): phosphorothioate bond
aaauaacucacuauaauuann

SEQ ID NO: 82 - RNA, 21 nt, Artificial Sequence
  synthesized siRNA antisense sequence
  misc_feature (1)..(1): um
  misc_feature (5)..(5): um
  misc_feature (7)..(7): um
  misc_feature (15)..(15): um
  misc_feature (20)..(21): n=deoxythymidine
  misc_feature (20)..(21): phosphorothioate bond
uaauuauagugaguuauuunn

SEQ ID NO: 83 - RNA, 21 nt, Artificial Sequence
  synthesized siRNA sense sequence
  misc_feature (2)..(2): um
  misc_feature (4)..(5): um
  misc_feature (10)..(10): um
  misc_feature (12)..(12): um
  misc_feature (13)..(13): cm
  misc_feature (14)..(14): um
  misc_feature (16)..(16): cm
  misc_feature (17)..(17): um
  misc_feature (19)..(19): um
  misc_feature (20)..(21): n=deoxythymidine
  misc_feature (20)..(21): phosphorothioate bond
guguuaaaaugucugcugunn

SEQ ID NO: 84 - RNA, 21 nt, Artificial Sequence
  synthesized siRNA antisense sequence
  misc_feature (2)..(2): cm
  misc_feature (5)..(5): cm
  misc_feature (9)..(9): cm
  misc_feature (14)..(14): um
  misc_feature (17)..(17): cm
  misc_feature (20)..(21): n=deoxythymidine
  misc_feature (20)..(21): phosphorothioate bond
acagcagacauuuuaacacnn

SEQ ID NO: 85 - RNA, 21 nt, Artificial Sequence
  synthesized siRNA sense sequence
  misc_feature (5)..(5): um
  misc_feature (7)..(11): um
  misc_feature (13)..(13): um
  misc_feature (14)..(14): cm
  misc_feature (18)..(18): um
  misc_feature (20)..(21): n=deoxythymidine
  misc_feature (20)..(21): phosphorothioate bond
aaaauguuuuugucaaguann

SEQ ID NO: 86 - RNA, 21 nt, Artificial Sequence
  synthesized siRNA antisense sequence
  misc_feature (1)..(1): um
  misc_feature (8)..(8): cm
  misc_feature (14)..(14): cm
  misc_feature (20)..(21): n=deoxythymidine
  misc_feature (20)..(21): phosphorothioate bond
uacuugacaaaaacauuuunn

SEQ ID NO: 87 - RNA, 21 nt, Artificial Sequence
  synthesized siRNA sense sequence
  misc_feature (1)..(1): cm
  misc_feature (2)..(3): um
  misc_feature (5)..(5): cm
  misc_feature (7)..(7): cm
  misc_feature (8)..(8): um
  misc_feature (12)..(12): um
  misc_feature (14)..(14): cm
  misc_feature (15)..(16): um
  misc_feature (17)..(17): cm
  misc_feature (20)..(21): n=deoxythymidine
  misc_feature (20)..(21): phosphorothioate bond
cuuacgcugaguacuucgann

SEQ ID NO: 88 - RNA, 21 nt, Artificial Sequence
  synthesized siRNA antisense sequence
  misc_feature (7)..(7): um
  misc_feature (11)..(11): cm
  misc_feature (16)..(16): um
  misc_feature (20)..(21): n=deoxythymidine
  misc_feature (20)..(21): phosphorothioate bond
ucgaaguacucagcguaagnn

SEQ ID NO: 89 - RNA, 21 nt, Artificial Sequence
  synthesized siRNA sense sequence
  misc_feature (4)..(4): cm
  misc_feature (10)..(10): um
  misc_feature (12)..(13): um
  misc_feature (14)..(14): cm
  misc_feature (20)..(21): n=deoxythymidine
  misc_feature (20)..(21): phosphorothioate bond
aggcaaagguguucaaagann

SEQ ID NO: 90 - RNA, 21 nt, Artificial Sequence
  synthesized siRNA antisense sequence
  misc_feature (9)..(9): cm
  misc_feature (20)..(21): n=deoxythymidine
  misc_feature (20)..(21): phosphorothioate bond
ucuuugaacaccuuugccunn

SEQ ID NO: 91 - RNA, 19 nt, Artificial Sequence
  synthesized siRNA sense sequence
  misc_feature (4)..(4): cm
  misc_feature (10)..(10): um
  misc_feature (12)..(13): um
  misc_feature (14)..(14): cm
aggcaaagguguucaaaga

SEQ ID NO: 92 - RNA, 19 nt, Artificial Sequence
  synthesized siRNA antisense sequence
  misc_feature (9)..(9): cm
ucuuugaacaccuuugccu

SEQ ID NO: 93 - RNA, 21 nt, Artificial Sequence
  synthesized siRNA sense sequence
  misc_feature (1)..(2): phosphorothioate bond
  misc_feature (4)..(4): cm
  misc_feature (10)..(10): um
  misc_feature (10)..(21): um
  misc_feature (12)..(13): um
  misc_feature (14)..(14): cm
aggcaaagguguucaaagauu

SEQ ID NO: 94 - RNA, 21 nt, Artificial Sequence
  synthesized siRNA antisense sequence
  misc_feature (1)..(2): phosphorothioate bond
  misc_feature (9)..(9): cm
  misc_feature (20)..(21): um
ucuuugaacaccuuugccuuu

SEQ ID NO: 95 - RNA, 21 nt, Artificial Sequence
  synthesized siRNA sense sequence
  misc_feature (1)..(2): phosphorothioate bond
  misc_feature (2)..(3): phosphorothioate bond
  misc_feature (4)..(4): cm
  misc_feature (10)..(10): um
  misc_feature (12)..(13): um
  misc_feature (14)..(14): cm
  misc_feature (20)..(21): um
aggcaaagguguucaaagauu

SEQ ID NO: 96 - RNA, 21 nt, Artificial Sequence
  synthesized siRNA antisense sequence
  misc_feature (1)..(3): phosphorothioate bond
  misc_feature (9)..(9): cm
  misc_feature (20)..(21): um
ucuuugaacaccuuugccuuu

SEQ ID NO: 97 - RNA, 21 nt, Artificial Sequence
  synthesized siRNA sense sequence
  misc_feature (1)..(2): phosphorothioate bond
  misc_feature (4)..(4): cm
  misc_feature (10)..(10): um
  misc_feature (12)..(13): um
  misc_feature (14)..(14): cm
  misc_feature (19)..(20): phosphorothioate bond
  misc_feature (20)..(21): phosphorothioate bond
  misc_feature (20)..(21): um
aggcaaagguguucaaagauu

SEQ ID NO: 98 - RNA, 21 nt, Artificial Sequence
  synthesized siRNA antisense sequence
  misc_feature (1)..(2): phosphorothioate bond
  misc_feature (9)..(9): cm
  misc_feature (19)..(21): phosphorothioate bond
  misc_feature (20)..(21): um
ucuuugaacaccuuugccuuu

SEQ ID NO: 99 - RNA, 19 nt, Artificial Sequence
  synthesized siRNA sense sequence
  misc_feature (4)..(4): cm
  misc_feature (10)..(10): um
  misc_feature (12)..(13): um
  misc_feature (14)..(14): cm
aggcaaagguguucaaaga

SEQ ID NO: 100 - RNA, 21 nt, Artificial Sequence
  synthesized siRNA antisense sequence
  misc_feature (9)..(9): cm
  misc_feature (20)..(21): um
ucuuugaacaccuuugccuuu

SEQ ID NO: 101 - RNA, 21 nt, Artificial Sequence
  synthesized siRNA sense sequence
  misc_feature (2)..(2): um
  misc_feature (6)..(6): cm
  misc_feature (12)..(12): um
  misc_feature (14)..(15): um
  misc_feature (16)..(16): cm
auaggcaaagguguucaaaga

SEQ ID NO: 102 - RNA, 21 nt, Artificial Sequence
  synthesized siRNA antisense sequence
  misc_feature (9)..(9): cm
  misc_feature (20)..(21): um
ucuuugaacaccuuugccuuu

SEQ ID NO: 103 - skipped

SEQ ID NO: 104 - RNA, 21 nt, Artificial Sequence
  synthesized siRNA antisense sequence
  misc_feature (9)..(9): cm
  misc_feature (20)..(21): um
ucuuugaacaccuuugccuuu

SEQ ID NO: 105 - skipped

SEQ ID NO: 106 - RNA, 21 nt, Artificial Sequence
  synthesized siRNA antisense sequence
  misc_feature (8)..(8): cm
  misc_feature (20)..(21): n=deoxythymidine
  misc_feature (20)..(21): phosphorothioate bond
cuuugaacaccuuugccuunn

SEQ ID NO: 107 - RNA, 19 nt, Artificial Sequence
  synthesized siRNA sense sequence
  misc_feature (5)..(5): cm
  misc_feature (11)..(11): um
  misc_feature (13)..(14): um
  misc_feature (15)..(15): cm
aaggcaaagguguucaaag

SEQ ID NO: 108 - RNA, 19 nt, Artificial Sequence
  synthesized siRNA antisense sequence
  misc_feature (8)..(8): cm
cuuugaacaccuuugccuu

SEQ ID NO: 109 - RNA, 21 nt, Artificial Sequence
  synthesized siRNA sense sequence
  misc_feature (9)..(9): cm
  misc_feature (10)..(10): um
  misc_feature (12)..(14): um
  misc_feature (16)..(16): cm
  misc_feature (19)..(19): cm
  misc_feature (20)..(21): n=deoxythymidine
  misc_feature (20)..(21): phosphorothioate bond
gaaaggaacuguuuacaacnn

SEQ ID NO: 110 - RNA, 21 nt, Artificial Sequence
  synthesized siRNA antisense sequence
  misc_feature (5)..(5): um
  misc_feature (9)..(9): cm
  misc_feature (20)..(21): n=deoxythymidine
  misc_feature (20)..(21): phosphorothioate bond
guuguaaacaguuccuuucnn

SEQ ID NO: 111 - RNA, 19 nt, Artificial Sequence
  synthesized siRNA sense sequence
  misc_feature (9)..(9): cm
  misc_feature (10)..(10): um
  misc_feature (13)..(15): um
  misc_feature (17)..(17): cm
  misc_feature (19)..(19): cm
gaaaggaacuguuuacaac

SEQ ID NO: 112 - RNA, 19 nt, Artificial Sequence
  synthesized siRNA antisense sequence
  misc_feature (5)..(5): um
  misc_feature (9)..(9): cm
guuguaaacaguuccuuuc

SEQ ID NO: 113 - RNA, 21 nt, Artificial Sequence
  synthesized siRNA sense sequence
  misc_feature (1)..(1): um
  misc_feature (4)..(4): um
  misc_feature (6)..(6): um
  misc_feature (8)..(8): um
  misc_feature (10)..(10): um
  misc_feature (12)..(13): um
  misc_feature (15)..(15): cm
  misc_feature (16)..(16): um
  misc_feature (19)..(19): um
  misc_feature (20)..(21): n=deoxythymidine
  misc_feature (20)..(21): phosphorothioate bond
ugguauauguguugcugaunn

SEQ ID NO: 114 - RNA, 21 nt, Artificial Sequence
  synthesized siRNA antisense sequence
  misc_feature (3)..(3): cm
  misc_feature (6)..(6): cm
  misc_feature (9)..(9): cm
  misc_feature (11)..(11): cm
  misc_feature (13)..(13): um
  misc_feature (15)..(15): um
  misc_feature (18)..(18): cm
  misc_feature (20)..(21): n=deoxythymidine
  misc_feature (20)..(21): phosphorothioate bond
aucagcaacacauauaccann

SEQ ID NO: 115 - RNA, 19 nt, Artificial Sequence
  synthesized siRNA sense sequence
  misc_feature (1)..(1): um
  misc_feature (4)..(4): um
  misc_feature (6)..(6): um
  misc_feature (8)..(8): um
  misc_feature (10)..(10): um
  misc_feature (12)..(13): um
  misc_feature (15)..(15): cm
  misc_feature (16)..(16): um
  misc_feature (19)..(19): um
ugguauauguguugcugau

SEQ ID NO: 116 - RNA, 19 nt, Artificial Sequence
  synthesized siRNA antisense sequence
  misc_feature (3)..(3): cm
  misc_feature (6)..(6): cm
  misc_feature (9)..(9): cm
  misc_feature (11)..(11): cm
  misc_feature (13)..(13): um
  misc_feature (15)..(15): um
  misc_feature (18)..(18): cm
aucagcaacacauauacca

SEQ ID NO: 117 - RNA, 21 nt, Artificial Sequence
  synthesized siRNA sense sequence
  misc_feature (1)..(1): um
  misc_feature (1)..(2): phosphorothioate bond
  misc_feature (4)..(4): um
  misc_feature (6)..(6): um
  misc_feature (8)..(8): um
  misc_feature (10)..(10): um
  misc_feature (12)..(13): um
  misc_feature (15)..(15): cm
  misc_feature (16)..(16): um
  misc_feature (19)..(21): um
ugguauauguguugcugauuu

SEQ ID NO: 118 - RNA, 21 nt, Artificial Sequence
  synthesized siRNA antisense sequence
  misc_feature (1)..(2): phosphorothioate bond
  misc_feature (3)..(3): cm
  misc_feature (6)..(6): cm
  misc_feature (9)..(9): cm
  misc_feature (11)..(11): cm
  misc_feature (13)..(13): um
  misc_feature (15)..(15): um
  misc_feature (18)..(18): cm
  misc_feature (20)..(21): um
aucagcaacacauauaccauu

SEQ ID NO: 119 - RNA, 21 nt, Artificial Sequence
  synthesized siRNA sense sequence
  misc_feature (1)..(3): phosphorothioate bond
  misc_feature (1)..(1): um
  misc_feature (4)..(4): um
  misc_feature (6)..(6): um
  misc_feature (8)..(8): um
  misc_feature (10)..(10): um
  misc_feature (12)..(13): um
  misc_feature (15)..(15): cm
  misc_feature (16)..(16): um
  misc_feature (19)..(21): um
ugguauauguguugcugauuu

SEQ ID NO: 120 - RNA, 21 nt, Artificial Sequence
  synthesized siRNA antisense sequence
  misc_feature (1)..(3): phosphorothioate bond
  misc_feature (3)..(3): cm
  misc_feature (6)..(6): cm
  misc_feature (9)..(9): cm
  misc_feature (11)..(11): cm
  misc_feature (13)..(13): um
  misc_feature (15)..(15): um
  misc_feature (18)..(18): cm
  misc_feature (20)..(21): um
aucagcaacacauauaccauu

SEQ ID NO: 121 - RNA, 21 nt, Artificial Sequence
  synthesized siRNA sense sequence
  misc_feature (1)..(1): um
  misc_feature (1)..(2): phosphorothioate bond
  misc_feature (4)..(4): um
  misc_feature (6)..(6): um
  misc_feature (8)..(8): um
  misc_feature (10)..(10): um
  misc_feature (12)..(13): um
  misc_feature (15)..(15): cm
  misc_feature (16)..(16): um
  misc_feature (19)..(21): um
  misc_feature (19)..(21): phosphorothioate bond
ugguauauguguugcugauuu

SEQ ID NO: 122 - RNA, 21 nt, Artificial Sequence
  synthesized siRNA antisense sequence
  misc_feature (1)..(2): phosphorothioate bond
  misc_feature (3)..(3): cm
  misc_feature (6)..(6): cm
  misc_feature (9)..(9): cm
  misc_feature (11)..(11): cm
  misc_feature (13)..(13): um
  misc_feature (15)..(15): um
  misc_feature (18)..(18): cm
  misc_feature (19)..(21): phosphorothioate bond
  misc_feature (20)..(21): um
aucagcaacacauauaccauu

SEQ ID NO: 123 - RNA, 19 nt, Artificial Sequence
  synthesized siRNA sense sequence
  misc_feature (1)..(1): um
  misc_feature (4)..(4): um
  misc_feature (6)..(6): um
  misc_feature (8)..(8): um
  misc_feature (10)..(10): um
  misc_feature (12)..(13): um
  misc_feature (15)..(15): cm
  misc_feature (16)..(16): um
  misc_feature (19)..(19): um
ugguauauguguugcugau

SEQ ID NO: 124 - RNA, 21 nt, Artificial Sequence
  synthesized siRNA antisense sequence
  misc_feature (3)..(3): cm
  misc_feature (6)..(6): cm
  misc_feature (9)..(9): cm
  misc_feature (11)..(11): cm
  misc_feature (13)..(13): um
  misc_feature (15)..(15): um
  misc_feature (18)..(18): cm
  misc_feature (20)..(21): um
aucagcaacacauauaccauu

SEQ ID NO: 125 - RNA, 21 nt, Artificial Sequence
  synthesized siRNA sense sequence
  misc_feature (2)..(3): um
  misc_feature (6)..(6): um
  misc_feature (8)..(8): um
  misc_feature (10)..(10): um
  misc_feature (12)..(12): um
  misc_feature (14)..(15): um
  misc_feature (17)..(17): cm
  misc_feature (18)..(18): um
  misc_feature (21)..(21): um
auugguauauguguugcugau

SEQ ID NO: 126 - RNA, 21 nt, Artificial Sequence
  synthesized siRNA antisense sequence
  misc_feature (3)..(3): cm
  misc_feature (6)..(6): cm
  misc_feature (9)..(9): cm
  misc_feature (11)..(11): cm
  misc_feature (13)..(13): um
  misc_feature (15)..(15): um
  misc_feature (18)..(18): cm
  misc_feature (20)..(21): um
aucagcaacacauauaccauu

SEQ ID NO: 127 - RNA, 22 nt, Artificial Sequence
  synthesized siRNA sense sequence
  misc_feature (1)..(1): um
  misc_feature (3)..(4): um
  misc_feature (7)..(7): um
  misc_feature (9)..(9): um
  misc_feature (11)..(11): um
  misc_feature (13)..(13): um
  misc_feature (15)..(16): um
  misc_feature (18)..(18): cm
  misc_feature (19)..(19): um
  misc_feature (22)..(22): um
uauugguauauguguugcugau

SEQ ID NO: 128 - RNA, 21 nt, Artificial Sequence
  synthesized siRNA antisense sequence
  misc_feature (3)..(3): cm
  misc_feature (6)..(6): cm
  misc_feature (9)..(9): cm
  misc_feature (11)..(11): cm
  misc_feature (13)..(13): um
  misc_feature (15)..(15): um
  misc_feature (18)..(18): cm
  misc_feature (20)..(21): um
aucagcaacacauauaccauu

SEQ ID NO: 129 - RNA, 23 nt, Artificial Sequence
  synthesized siRNA sense sequence
  misc_feature (1)..(1): um
  misc_feature (3)..(4): um
  misc_feature (7)..(7): um
  misc_feature (9)..(9): um
  misc_feature (11)..(11): um
  misc_feature (13)..(13): um
  misc_feature (15)..(16): um
  misc_feature (18)..(18): cm
  misc_feature (19)..(19): um
  misc_feature (22)..(22): um
  misc_feature (23)..(23): n=Inverted deoxythymidine
uauugguauauguguugcugaun

SEQ ID NO: 130 - RNA, 21 nt, Artificial Sequence
  synthesized siRNA antisense sequence
  misc_feature (3)..(3): cm
  misc_feature (6)..(6): cm
  misc_feature (9)..(9): cm
  misc_feature (11)..(11): cm
  misc_feature (13)..(13): um
  misc_feature (15)..(15): um
  misc_feature (18)..(18): cm
  misc_feature (20)..(21): um
aucagcaacacauauaccauu

SEQ ID NO: 131 - RNA, 21 nt, Artificial Sequence
  synthesized siRNA sense sequence
  misc_feature (1)..(1): um
  misc_feature (4)..(4): um
  misc_feature (6)..(6): um
  misc_feature (8)..(8): um
  misc_feature (10)..(10): um
  misc_feature (12)..(13): um
  misc_feature (16)..(16): um
  misc_feature (19)..(21): um
ugguauauguguugcugauuu

SEQ ID NO: 132 - RNA, 21 nt, Artificial Sequence
  synthesized siRNA antisense sequence
  misc_feature (3)..(3): cm
  misc_feature (6)..(6): cm
  misc_feature (9)..(9): cm
  misc_feature (11)..(11): cm
  misc_feature (13)..(13): um
  misc_feature (15)..(15): um
  misc_feature (18)..(18): cm
  misc_feature (20)..(21): um
aucagcaacacauauaccauu

SEQ ID NO: 133 - RNA, 21 nt, Artificial Sequence
  synthesized siRNA sense sequence
  misc_feature (1)..(1): um
  misc_feature (4)..(4): um
  misc_feature (6)..(6): um
  misc_feature (8)..(8): um
  misc_feature (10)..(10): um
  misc_feature (12)..(13): um
  misc_feature (15)..(15): cm
  misc_feature (19)..(21): um
ugguauauguguugcugauuu

SEQ ID NO: 134 - RNA, 21 nt, Artificial Sequence
  synthesized siRNA antisense sequence
  misc_feature (3)..(3): cm
  misc_feature (6)..(6): cm
  misc_feature (9)..(9): cm
  misc_feature (11)..(11): cm
  misc_feature (13)..(13): um
  misc_feature (15)..(15): um
  misc_feature (18)..(18): cm
  misc_feature (20)..(21): um
aucagcaacacauauaccauu

SEQ ID NO: 135 - RNA, 20 nt, Artificial Sequence
  synthesized siRNA sense sequence
  misc_feature (2)..(3): um
  misc_feature (6)..(6): um
  misc_feature (8)..(8): um
  misc_feature (10)..(10): um
  misc_feature (12)..(12): um
  misc_feature (14)..(15): um
  misc_feature (17)..(17): cm
  misc_feature (18)..(18): um
auugguauauguguugcuga

SEQ ID NO: 136 - RNA, 20 nt, Artificial Sequence
  synthesized siRNA antisense sequence
  misc_feature (2)..(2): cm
  misc_feature (5)..(5): cm
  misc_feature (8)..(8): cm
  misc_feature (10)..(10): cm
  misc_feature (12)..(12): um
  misc_feature (14)..(14): um
  misc_feature (17)..(17): cm
  misc_feature (19)..(20): um
ucagcaacacauauaccauu

SEQ ID NO: 137 - RNA, 20 nt, Artificial Sequence
  synthesized siRNA sense sequence
  misc_feature (2)..(2): um
  misc_feature (5)..(5): um
  misc_feature (7)..(7): um
  misc_feature (9)..(9): um
  misc_feature (11)..(11): um
  misc_feature (13)..(14): um
  misc_feature (16)..(16): cm
  misc_feature (17)..(17): um
  misc_feature (20)..(20): um
augguauauguguugcugau

SEQ ID NO: 138 - RNA, 20 nt, Artificial Sequence
  synthesized siRNA antisense sequence
  misc_feature (3)..(3): cm
  misc_feature (6)..(6): cm
  misc_feature (9)..(9): cm
  misc_feature (11)..(11): cm
  misc_feature (13)..(13): um
  misc_feature (15)..(15): um
  misc_feature (18)..(18): cm
  misc_feature (19)..(20): um
aucagcaacacauauaccuu

SEQ ID NO: 139 - RNA, 19 nt, Artificial Sequence
  synthesized siRNA sense sequence
  misc_feature (1)..(1): um
  misc_feature (4)..(4): um
  misc_feature (6)..(6): um
  misc_feature (8)..(8): um
  misc_feature (10)..(10): um
  misc_feature (12)..(13): um
  misc_feature (15)..(15): cm
  misc_feature (16)..(16): um
  misc_feature (19)..(19): um
ugguauauguguugcugau

SEQ ID NO: 140 - RNA, 23 nt, Artificial Sequence
  synthesized siRNA antisense sequence
  misc_feature (3)..(3): cm
  misc_feature (6)..(6): cm
  misc_feature (9)..(9): cm
  misc_feature (11)..(11): cm
  misc_feature (13)..(13): um
  misc_feature (15)..(15): um
  misc_feature (18)..(18): cm
  misc_feature (20)..(23): um
aucagcaacacauauaccauuuu

SEQ ID NO: 141 - RNA, 19 nt, Artificial Sequence
  synthesized siRNA sense sequence
  misc_feature (1)..(1): um
  misc_feature (4)..(4): um
  misc_feature (6)..(6): um
  misc_feature (8)..(8): um
  misc_feature (10)..(10): um
  misc_feature (12)..(13): um
  misc_feature (16)..(16): um
  misc_feature (19)..(19): um
ugguauauguguugcugau

SEQ ID NO: 142 - RNA, 23 nt, Artificial Sequence
  synthesized siRNA antisense sequence
  misc_feature (3)..(3): cm
  misc_feature (6)..(6): cm
  misc_feature (9)..(9): cm
  misc_feature (11)..(11): cm
  misc_feature (13)..(13): um
  misc_feature (15)..(15): um
  misc_feature (18)..(18): cm
  misc_feature (20)..(23): um
aucagcaacacauauaccauuuu

SEQ ID NO: 143 - RNA, 19 nt, Artificial Sequence
  synthesized siRNA sense sequence
  misc_feature (1)..(1): um
  misc_feature (4)..(4): um
  misc_feature (6)..(6): um
  misc_feature (8)..(8): um
  misc_feature (10)..(10): um
  misc_feature (12)..(13): um
  misc_feature (16)..(16): um
  misc_feature (19)..(19): um
ugguauauguguugcugau

SEQ ID NO: 144 - RNA, 23 nt, Artificial Sequence
  synthesized siRNA antisense sequence
  misc_feature (3)..(3): cm
  misc_feature (6)..(6): cm
  misc_feature (9)..(9): cm
  misc_feature (11)..(11): cm
  misc_feature (13)..(13): um
  misc_feature (15)..(15): um
  misc_feature (18)..(18): cm
  misc_feature (20)..(21): um
  misc_feature (22)..(23): 2′-o-methyladenosine
aucagcaacacauauaccauuaa

SEQ ID NO: 145 - RNA, 19 nt, Artificial Sequence
  synthesized siRNA sense sequence
  misc_feature (1)..(1): um
  misc_feature (4)..(4): um
  misc_feature (6)..(6): um
  misc_feature (8)..(8): um
  misc_feature (10)..(10): um
  misc_feature (12)..(13): um
  misc_feature (16)..(16): um
  misc_feature (19)..(19): um
ugguauauguguugcugau

SEQ ID NO: 146 - RNA, 23 nt, Artificial Sequence
  synthesized siRNA antisense sequence
  misc_feature (3)..(3): cm
  misc_feature (6)..(6): cm
  misc_feature (9)..(9): cm
  misc_feature (11)..(11): cm
  misc_feature (13)..(13): um
  misc_feature (15)..(15): um
  misc_feature (18)..(18): cm
  misc_feature (20)..(21): um
  misc_feature (21)..(23): phosphorothioate bond
  misc_feature (22)..(23): 2′-o-methyladenosine
aucagcaacacauauaccauuaa

SEQ ID NO: 147 - RNA, 19 nt, Artificial Sequence
  synthesized siRNA sense sequence
  misc_feature (1)..(1): um
  misc_feature (4)..(4): um
  misc_feature (6)..(6): um
  misc_feature (8)..(8): um
  misc_feature (10)..(10): um
  misc_feature (12)..(13): um
  misc_feature (16)..(16): um
  misc_feature (19)..(19): um
ugguauauguguugcugau

SEQ ID NO: 148 - RNA, 23 nt, Artificial Sequence
  synthesized siRNA antisense sequence
  misc_feature (3)..(3): cm
  misc_feature (6)..(6): cm
  misc_feature (9)..(9): cm
  misc_feature (11)..(11): cm
  misc_feature (13)..(13): um
  misc_feature (15)..(15): um
  misc_feature (18)..(18): cm
  misc_feature (19)..(21): phosphorothioate bond
  misc_feature (20)..(21): um
  misc_feature (22)..(23): 2′-o-methyladenosine
aucagcaacacauauaccauuaa

SEQ ID NO: 149 - RNA, 19 nt, Artificial Sequence
  synthesized siRNA sense sequence
  misc_feature (1)..(1): um
  misc_feature (4)..(4): um
  misc_feature (6)..(6): um
  misc_feature (8)..(8): um
  misc_feature (10)..(10): um
  misc_feature (12)..(13): um
  misc_feature (16)..(16): um
  misc_feature (19)..(19): um
ugguauauguguugcugau

SEQ ID NO: 150 - RNA, 23 nt, Artificial Sequence
  synthesized siRNA antisense sequence
  misc_feature (1)..(1): 2′-o-methyladenosine
  misc_feature (1)..(2): phosphorothioate bond
  misc_feature (3)..(3): cm
  misc_feature (6)..(6): cm
  misc_feature (9)..(9): cm
  misc_feature (11)..(11): cm
  misc_feature (13)..(13): um
  misc_feature (15)..(15): um
  misc_feature (18)..(18): cm
  misc_feature (19)..(21): phosphorothioate bond
  misc_feature (20)..(21): um
  misc_feature (22)..(23): 2′-o-methyladenosine
aucagcaacacauauaccauuaa

SEQ ID NO: 151 - RNA, 19 nt, Artificial Sequence
  synthesized siRNA sense sequence
  misc_feature (1)..(1): um
  misc_feature (4)..(4): um
  misc_feature (6)..(6): um
  misc_feature (8)..(8): um
  misc_feature (10)..(10): um
  misc_feature (12)..(13): um
  misc_feature (16)..(16): um
  misc_feature (19)..(19): um
ugguauauguguugcugau

SEQ ID NO: 152 - RNA, 23 nt, Artificial Sequence
  synthesized siRNA antisense sequence
  misc_feature (1)..(1): 2′-o-methyladenosine
  misc_feature (3)..(3): cm
  misc_feature (6)..(6): cm
  misc_feature (9)..(9): cm
  misc_feature (11)..(11): cm
  misc_feature (13)..(13): um
  misc_feature (15)..(15): um
  misc_feature (18)..(18): cm
  misc_feature (20)..(23): um
aucagcaacacauauaccauuuu

SEQ ID NO: 153 - RNA, 19 nt, Artificial Sequence
  synthesized siRNA sense sequence
  misc_feature (1)..(1): um
  misc_feature (1)..(2): phosphorothioate bond
  misc_feature (4)..(4): um
  misc_feature (6)..(6): um
  misc_feature (8)..(8): um
  misc_feature (10)..(10): um
  misc_feature (12)..(13): um
  misc_feature (16)..(16): um
  misc_feature (19)..(19): um
ugguauauguguugcugau

SEQ ID NO: 154 - RNA, 23 nt, Artificial Sequence
  synthesized siRNA antisense sequence
  misc_feature (3)..(3): cm
  misc_feature (6)..(6): cm
  misc_feature (9)..(9): cm
  misc_feature (11)..(11): cm
  misc_feature (13)..(13): um
  misc_feature (15)..(15): um
  misc_feature (18)..(18): cm
  misc_feature (19)..(21): phosphorothioate bond
  misc_feature (20)..(21): um
  misc_feature (22)..(23): 2′-o-methyladenosine
aucagcaacacauauaccauuaa

SEQ ID NO: 155 - RNA, 21 nt, Artificial Sequence
  synthesized siRNA sense sequence
  misc_feature (2)..(2): um
  misc_feature (5)..(5): cm
  misc_feature (6)..(6): um
  misc_feature (7)..(7): cm
  misc_feature (9)..(9): cm
  misc_feature (10)..(10): um
  misc_feature (12)..(12): um
  misc_feature (15)..(16): um
  misc_feature (18)..(18): cm
  misc_feature (19)..(19): um
  misc_feature (20)..(21): n=deoxythymidine
  misc_feature (20)..(21): phosphorothioate bond
auaacucacuauaauuacunn

SEQ ID NO: 156 - RNA, 21 nt, Artificial Sequence
  synthesized siRNA antisense sequence
  misc_feature (3)..(3): um
  misc_feature (7)..(7): um
  misc_feature (9)..(9): um
  misc_feature (17)..(17): um
  misc_feature (20)..(21): n=deoxythymidine
  misc_feature (20)..(21): phosphorothioate bond
aguaauuauagugaguuaunn

SEQ ID NO: 157 - RNA, 19 nt, Artificial Sequence
  synthesized siRNA sense sequence
  misc_feature (2)..(2): um
  misc_feature (5)..(5): cm
  misc_feature (6)..(6): um
  misc_feature (7)..(7): cm
  misc_feature (9)..(9): cm
  misc_feature (10)..(10): um
  misc_feature (12)..(12): um
  misc_feature (15)..(16): um
  misc_feature (18)..(18): cm
  misc_feature (19)..(19): um
auaacucacuauaauuacu

SEQ ID NO: 158 - RNA, 19 nt, Artificial Sequence
  synthesized siRNA antisense sequence
  misc_feature (3)..(3): um
  misc_feature (7)..(7): um
  misc_feature (9)..(9): um
  misc_feature (17)..(17): um
aguaauuauagugaguuau

SEQ ID NO: 159 - RNA, 21 nt, Artificial Sequence
  synthesized siRNA sense sequence
  misc_feature (1)..(1): um
  misc_feature (4)..(4): um
  misc_feature (6)..(6): um
  misc_feature (8)..(8): um
  misc_feature (10)..(10): um
  misc_feature (12)..(13): um
  misc_feature (15)..(15): cm
  misc_feature (16)..(16): um
  misc_feature (19)..(21): um
ugguauauguguugcugauuu

SEQ ID NO: 160 - RNA, 21 nt, Artificial Sequence
  synthesized siRNA antisense sequence
  misc_feature (3)..(3): cm
  misc_feature (6)..(6): cm
  misc_feature (9)..(9): cm
  misc_feature (11)..(11): cm
  misc_feature (13)..(13): um
  misc_feature (15)..(15): um
  misc_feature (18)..(18): cm
  misc_feature (20)..(21): um
aucagcaacacauauaccauu

SEQ ID NO: 161 - RNA, 21 nt, Artificial Sequence
  synthesized siRNA sense sequence
  misc_feature (4)..(4): cm
  misc_feature (10)..(10): um
  misc_feature (12)..(13): um
  misc_feature (14)..(14): cm
  misc_feature (20)..(21): um
aggcaaagguguucaaagauu

SEQ ID NO: 162 - RNA, 21 nt, Artificial Sequence
  synthesized siRNA antisense sequence
  misc_feature (9)..(9): cm
  misc_feature (20)..(21): um
ucuuugaacaccuuugccuuu

SEQ ID NO: 163 - RNA, 21 nt, Artificial Sequence
  synthesized siRNA sense sequence
  misc_feature (9)..(9): cm
  misc_feature (10)..(10): um
  misc_feature (12)..(14): um
  misc_feature (16)..(16): cm
  misc_feature (19)..(19): cm
  misc_feature (20)..(21): um
gaaaggaacuguuuacaacuu

SEQ ID NO: 164 - RNA, 21 nt, Artificial Sequence
  synthesized siRNA antisense sequence
  misc_feature (5)..(5): um
  misc_feature (9)..(9): cm
  misc_feature (20)..(21): um
guuguaaacaguuccuuucuu

SEQ ID NO: 165 - RNA, 21 nt, Artificial Sequence
  synthesized siRNA sense sequence
  misc_feature (2)..(2): cm
  misc_feature (3)..(3): um
  misc_feature (6)..(6): um
  misc_feature (8)..(8): um
  misc_feature (10)..(10): um
  misc_feature (12)..(12): um
  misc_feature (14)..(15): um
  misc_feature (17)..(17): cm
  misc_feature (18)..(18): um
  misc_feature (20)..(21): n=deoxythymidine
  misc_feature (20)..(21): phosphorothioate bond
acugguauauguguugcugnn

SEQ ID NO: 166 - RNA, 21 nt, Artificial Sequence
  synthesized siRNA antisense sequence
  misc_feature (1)..(1): cm
  misc_feature (4)..(4): cm
  misc_feature (7)..(7): cm
  misc_feature (9)..(9): cm
  misc_feature (11)..(11): um
  misc_feature (13)..(13): um
  misc_feature (16)..(16): cm
  misc_feature (20)..(21): n=deoxythymidine
  misc_feature (20)..(21): phosphorothioate bond
cagcaacacauauaccagunn

SEQ ID NO: 167 - RNA, 21 nt, Artificial Sequence
  synthesized siRNA sense sequence
  misc_feature (1)..(1): cm
  misc_feature (2)..(2): um
  misc_feature (5)..(5): um
  misc_feature (7)..(7): um
  misc_feature (9)..(9): um
  misc_feature (11)..(11): um
  misc_feature (13)..(14): um
  misc_feature (16)..(16): cm
  misc_feature (17)..(17): um
  misc_feature (20)..(21): n=deoxythymidine
  misc_feature (20)..(21): phosphorothioate bond
cugguauauguguugcugann

SEQ ID NO: 168 - RNA, 21 nt, Artificial Sequence
  synthesized siRNA antisense sequence
  misc_feature (2)..(2): cm
  misc_feature (5)..(5): cm
  misc_feature (8)..(8): cm
  misc_feature (10)..(10): cm
  misc_feature (12)..(12): um
  misc_feature (14)..(14): um
  misc_feature (17)..(17): cm
  misc_feature (20)..(21): n=deoxythymidine
  misc_feature (20)..(21): phosphorothioate bond
ucagcaacacauauaccagnn

SEQ ID NO: 169 - RNA, 21 nt, Artificial Sequence
  synthesized siRNA sense sequence
  misc_feature (3)..(3): um
  misc_feature (5)..(5): um
  misc_feature (7)..(7): um
  misc_feature (9)..(9): um
  misc_feature (11)..(12): um
  misc_feature (14)..(14): cm
  misc_feature (15)..(15): um
  misc_feature (18)..(18): um
  misc_feature (20)..(21): n=deoxythymidine
  misc_feature (20)..(21): phosphorothioate bond
gguauauguguugcugauann

SEQ ID NO: 170 - RNA, 21 nt, Artificial Sequence
  synthesized siRNA antisense sequence
  misc_feature (1)..(1): um
  misc_feature (4)..(4): cm
  misc_feature (7)..(7): cm
  misc_feature (10)..(10): cm
  misc_feature (12)..(12): cm
  misc_feature (14)..(14): um
  misc_feature (16)..(16): um
  misc_feature (20)..(21): n=deoxythymidine
  misc_feature (20)..(21): phosphorothioate bond
uaucagcaacacauauaccnn

SEQ ID NO: 171 - RNA, 21 nt, Artificial Sequence
  synthesized siRNA sense sequence
  misc_feature (2)..(2): um
  misc_feature (4)..(4): um
  misc_feature (6)..(6): um
  misc_feature (8)..(8): um
  misc_feature (10)..(11): um
  misc_feature (13)..(13): cm
  misc_feature (14)..(14): um
  misc_feature (17)..(17): um
  misc_feature (19)..(19): cm
  misc_feature (20)..(21): n=deoxythymidine
  misc_feature (20)..(21): phosphorothioate bond
guauauguguugcugauacnn

SEQ ID NO: 172 - RNA, 21 nt, Artificial Sequence
  synthesized siRNA antisense sequence
  misc_feature (2)..(2): um
  misc_feature (5)..(5): cm
  misc_feature (8)..(8): cm
  misc_feature (11)..(11): cm
  misc_feature (13)..(13): cm
  misc_feature (15)..(15): um
  misc_feature (17)..(17): um
  misc_feature (20)..(21): n=deoxythymidine
  misc_feature (20)..(21): phosphorothioate bond
guaucagcaacacauauacnn